(19) United States
(12) Patent Application Publication
(10) Pub. No.: US 2025/0064711 A1
(43) Pub. Date: Feb. 27, 2025

(54) COSMETIC COMPOSITION COMPRISING A POLYHYDROXYALKANOATE COPOLYMER BEARING A(N) (UN)SATURATED HYDROCARBON-BASED CHAIN AND A NATURAL RESIN

(71) Applicant: L'OREAL, PARIS (FR)

(72) Inventors: Angelina ROUDOT, CHEVILLY-LARUE (FR); Lyubov LUKYANOVA, CHEVILLY-LARUE (FR); Julien PORTAL, AULNAY-SOUS-BOIS (FR)

(21) Appl. No.: 18/723,263

(22) PCT Filed: Dec. 22, 2022
(86) PCT No.: PCT/EP2022/087647

§ 371 Date: Jun. 21, 2024

(30) Foreign Application Priority Data
Dec. 23, 2021 (FR) FR2114382

(51) Int. Cl. A61K 8/85; A61Q 1/04; A61Q 1/10; A61Q 5/00; A61Q 5/06

(52) U.S. Cl. CPC A61K 8/85; A61Q 1/04; A61Q 1/10; A61Q 5/006; A61Q 5/065; A61K 2800/54; A61K 2800/85; A61K 2800/95

(57) ABSTRACT

The present invention relates to a cosmetic composition comprising a) one or more polyhydroxyalkanoate (PHA) copolymers comprising at least two different repeating polymer units chosen from the units (A) and (B) below, and also the optical or geometrical isomers thereof, the organic or mineral acid or base salts thereof, and the solvates thereof such as hydrates: —[—O—CH(R¹)—CH₂—C(O)—]— unit (A); —[—O—CH(R²)—CH₂—C(O)—]— unit (B) In which polymer units (A) and (B): —R¹ and R² are as defined in the description; b) one or more natural resins.

The present invention relates to a cosmetic composition comprising a) at least one polyhydroxyalkanoate copolymer bearing (un)saturated hydrocarbon-based groups, b) at least one natural resin, c) optionally at least one fatty substance, and d) optionally at least one organic solvent other than c), and also to a process for treating keratin materials using such a composition.

It is known practice to use, in cosmetics, film-forming polymers which can be conveyed in organic media, such as hydrocarbon-based oils. Polymers are notably used as film-forming agents in makeup products such as mascaras, eyeliners, eyeshadows or lipsticks.

FR-A-2964663 describes a cosmetic composition comprising pigments coated with a C₃-C₂₁ polyhydroxyalkanoate, such as poly(hydroxybutyrate-co-hydroxyvalerate).

WO 2011/154508 describes a cosmetic composition comprising a 4-carboxy-2-pyrrolidinone ester derivative and a film-forming polymer which may be a polyhydroxyalkanoate, such as polyhydroxybutyrate, polyhydroxyvalerate and polyhydroxybutyrate-co-polyhydroxyvalerate.

US-A-2015/274972 describes a cosmetic composition comprising a thermoplastic resin, such as a polyhydroxyalkanoate, in aqueous dispersion and a silicone elastomer.

The majority of the polyhydroxyalkanoate copolymers are polymers derived from the polycondensation of polymeric repeating units that are for the most part identical and derived from the same carbon source or substrate. These documents do not describe the cosmetic use of copolymers derived from polycondensation using an aliphatic substrate or first carbon source, and at least one second substrate different from the first, comprising one or more (un)saturated hydrocarbon-based groups with polysaccharides. There is thus a need for a composition comprising polyhydroxyalkanoate copolymers which are lipophilic or soluble in a fatty phase. This makes it possible to obtain a film on keratin materials which has good cosmetic properties, notably good resistance to oils and to sebum, and also to be able to modify the gloss or the mattness.

The Applicant has discovered that polyhydroxyalkanoate copolymers bearing particular grafted or functionalized hydrocarbon-based groups, as defined below, can be readily used in fatty media, thus making it possible to obtain homogeneous compositions. The composition has good stability, notably after storage for 1 month at room temperature (25° C.). The composition, notably after having been applied to keratin materials, produces a film which has good cosmetic properties, good colour persistence without leaching for the composition, and also a matt or shiny appearance of the treated keratin materials.

Natural resins and balsams were known and used in the earliest human societies. The various uses ranged from surface protection and decoration (for example Japanese lacquers, used since the 3rd century B.C.) to preservation and mummification (Egypt), in sealants and adhesives, medicines and for cosmetic purposes (for example disinfectant sealing of wounds); and for ritual and ceremonial purposes (frankincense).

It is known practice to use resins, for instance rosins, as tack agents, which withstand oxidation and light in adhesives. Alkaline salts are also used in the paper industry for special papers and as emulsifiers emulsion polymerization (see, for example, US 2 174, U.S. Pat. Nos. 2,155,036 and 2,086,458). Elemi resins were previously used as plasticizers in alcohol-based varnishes and in combination with copal resins, but also in the pharmaceutical, cosmetic and perfumery industries. Incenses were widely used as fumigation resins for religious purposes, and for their disinfecting effects, for purifying air and chasing away unpleasant odours, or for purifying the bedroom of sick individuals, and similarly Siamese benzoin resin was used in religious ceremonies (Indian, Buddhist or Catholic liturgy) optionally combined with other resins. However, the resins are not known to be used with PHAs and to give persistence to keratin materials notably with respect to rubbing and external attacking factors such as water, sebum, sweat or oil.

In many conditions of use of film-forming materials on keratin materials, for instance in makeup or colouring applications, it is desirable to have, in addition to good resistance to water and oils, notably food oils such as olive oil, very good resistance to rubbing of the deposits of film-forming materials both to avoid transfer, for example onto clothing, and to maintain a homogeneous appearance of the deposits. If the resistance to rubbing is insufficient, the deposits obtained can quickly become very unsightly for consumers, in particular if these deposits are coloured as in makeup applications such as lipsticks, foundations or mascaras. In hair applications, the absence of resistance to rubbing is also very problematic in all colouring applications since it gives rise to transfer onto clothing and creates an unsightly appearance of the keratin fibres. There is thus a need to improve the persistence of PHAs conveyed in an aqueous phase.

There is thus a real need to obtain deposits of film-forming materials that are resistant to oils, notably food oils, and that are water-resistant and have very good resistance to rubbing.

When the deposit is coloured, these problems of resistance to humidity lead to a transfer of colour, for example onto clothing, which is in itself problematic and which makes the deposit very unsightly.

These problems are solved by the use of the compositions described hereinbelow, these compositions making it possible to significantly improve the resistance to rubbing of polyhydroxyalkanoate (PHA) copolymer(s). Furthermore, the compositions according to the invention make it possible to obtain, after deposition, a film on keratin materials which has good cosmetic properties, notably good resistance to oils and to sebum, and good water resistance, and also to be able to modify the gloss or the mattness.

Thus, the main subject of the present invention is a composition, notably a cosmetic composition, comprising:
a) one or more polyhydroxyalkanoate (PHA) copolymers comprising at least two different repeating polymer units chosen from the units (A) and (B) below, and also the optical or geometrical isomers thereof, the organic or mineral acid or base salts thereof, and the solvates thereof such as hydrates:

—[—O—CH(R¹)—CH₂—C(O)—]— unit (A)

—[—O—CH(R²)—CH₂—C(O)—]— unit (B)
in which polymer units (A) and (B):
   R¹ represents a saturated or unsaturated, linear or branched, non-cyclic hydrocarbon-based chain, or a saturated or unsaturated, aromatic or non-aromatic cyclic hydrocarbon-based chain, comprising from 5 to 28 carbon atoms; preferably, the hydrocarbon-based chain is chosen from i) linear or branched (C₅-C₂₈)alkyl, ii) linear or branched (C₅-C₂₈)alkenyl, iii) linear or branched (C₅-C₂₈)alkynyl; preferably, the hydrocarbon-based group is linear;
   said hydrocarbon-based chain being:
      optionally substituted with one or more atoms or groups chosen from: a) halogen such as chlorine or bromine, b) hydroxyl, c) thiol, d) (di)(C₁-C₄)(alkyl)amino, e) (thio)carboxy, f) (thio)carboxamide —C(O)—N(Rₐ)₂ or C(S)—N(Rₐ)₂, g) cyano, h) iso(thio)cyanate, i) (hetero)aryl such as phenyl or furyl, and j) (hetero)cycloalkyl such as anhydride, epoxide or dithiolane, k) cosmetic active agent; l) R—X with R representing a group chosen from α) cycloalkyl such as cyclohexyl, β) heterocycloalkyl such as sugar, preferably monosaccharide such as glucose, γ) (hetero)aryl such as phenyl, δ) cosmetic active agent, m) thiosulfate, and X representing a′) O, S, N(Rₐ) or Si(R_{b})(R_{c}), b′) S(O)ᵣ, or (thio)carbonyl, c′) or combinations of a′) with b′) such as (thio)ester, (thio)amide, (thio)urea or sulfonamide; Rₐ representing a hydrogen atom, or a (C₁-C₄)alkyl group or an aryl(C₁-C₄)alkyl group such as benzyl; preferably, Rₐ represents a hydrogen atom; R_{b} and R_{c}, which may be identical or different, represent a (C₁-C₄)alkyl or (C₁-C₄)alkoxy group, particularly only one substituent; preferably chosen from b) halogen, and j) such as epoxide; and/or
      optionally interrupted with one or more a′) heteroatoms such as O, S, N(Rₐ) and Si(R_{b})(R_{c}), b′) S(O)ᵣ, (thio)carbonyl, c′) or combinations of a′) with b′) such as (thio)ester, (thio)amide, (thio)urea, sulfonamide with r being equal to 1 or 2, Rₐ being as defined previously; preferably, Rₐ represents a hydrogen atom, R_{b} and R_{c} being as defined previously;
   R² represents a cyclic or non-cyclic, linear or branched, saturated or unsaturated hydrocarbon-based group comprising from 3 to 30 carbon atoms optionally substituted with one or more atoms or groups a) to m) and/or optionally interrupted with one or more heteroatoms or groups a′) to c′) as defined for R¹; in particular chosen from linear or branched (C₃-C₂₈)alkyl and linear or branched (C₃-C₂₈)alkenyl, in particular a linear hydrocarbon-based group, more particularly (C₄-C₂₀)alkyl or (C₄-C₂₀)alkenyl; preferably, the hydrocarbon-based group has a carbon number corresponding to the number of carbon atoms of the radical R¹ from which at least one carbon atom is subtracted, preferably corresponding to the number of carbon atoms of the radical R¹ from which two carbon atoms are subtracted;
b) one or more natural resins;
c) optionally one or more fatty substances, which are preferably liquid at 25° C. and at atmospheric pressure;
d) optionally one or more organic solvents other than c),
e) optionally water,
it being understood that:
   (A) is different from (B) and
preferably, the composition contains ingredients c)+d).

According to a variant, a composition can be a composition, preferably a cosmetic composition, comprising a) one or more PHA copolymers a) comprising one ore more following units (A), and also the optical or geometric isomers thereof, the organic or mineral acid or base salts thereof, and the solvates thereof such as hydrates:

—[—O—CH(R¹)—CH₂—C(O)—]— unit (A)
in which polymer units (A):
   R¹ represents a saturated or unsaturated, linear or branched, non-cyclic hydrocarbon-based chain, or a saturated or unsaturated, aromatic or non-aromatic cyclic hydrocarbon-based chain, comprising from 5 to 28 carbon atoms; preferably, the hydrocarbon-based chain is chosen from i) linear or branched (C₅-C₂₈)alkyl, ii) linear or branched (C₅-C₂₈)alkenyl, iii) linear or branched (C₅-C₂₈)alkynyl; preferably, the hydrocarbon-based group is linear;
   said hydrocarbon-based chain being:
      optionally substituted with one or more atoms or groups chosen from: a) halogen such as chlorine or bromine, b) hydroxyl, c) thiol, d) (di)(C₁-C₄)(alkyl)amino, e) (thio)carboxy, f) (thio)carboxamide —C(O)—N(Rₐ)₂ or C(S)—N(Rₐ)₂, g) cyano, h) iso(thio)cyanate, i) (hetero)aryl such as phenyl or furyl, and j) (hetero)cycloalkyl such as anhydride, epoxide or dithiolane, k) cosmetic active agent; l) R—X with R representing a group chosen from α) cycloalkyl such as cyclohexyl, β) heterocycloalkyl such as sugar, preferably monosaccharide such as glucose, γ) (hetero)aryl such as phenyl, δ) cosmetic active agent, m) thiosulfate, and X representing a′) O, S, N(Rₐ) or Si(R_{b})(R_{c}), b′) S(O)ᵣ, or (thio)carbonyl, c′) or combinations of a′) with b′) such as (thio)ester, (thio)amide, (thio)urea or sulfonamide; Rₐ representing a hydrogen atom, or a (C₁-C₄)alkyl group or an aryl(C₁-C₄)alkyl group such as benzyl; preferably, Rₐ represents a hydrogen atom; R_{b} and R_{c}, which may be identical or different, represent a (C₁-C₄)alkyl or (C₁-C₄)alkoxy group, particularly only one substituent; preferably chosen from b) halogen, and j) such as epoxide; and/or
      optionally interrupted with one or more a′) heteroatoms such as O, S, N(Rₐ) and Si(R_{b})(R_{c}), b′) S(O)ᵣ, (thio)carbonyl, c′) or combinations of a′) with b′) such as (thio)ester, (thio)amide, (thio)urea, sulfonamide with r being equal to 1 or 2, Rₐ being as defined previously; preferably, Rₐ represents a hydrogen atom, R_{b} and R_{c} being as defined previously; and
b) one or more natural resins;
c) optionally one or more fatty substances, which are preferably liquid at 25° C. and at atmospheric pressure;
d) optionally one or more organic solvents other than c),
e) optionally water,
it being understood that:
   (A) is different from (B) and
preferably, the composition contains ingredients c)+d).

Another object of the invention is the cosmetic use of a composition comprising a) one or more PHA copolymers as defined previously, b) one or more natural resins as defined previously, optionally c) one or more fatty substances as defined previously, d) optionally organic solvents other than c), and e) optionally water.

Another subject of the invention is a process for treating keratin materials, preferably α) keratin fibres, notably human keratin fibres such as the hair, or β) human skin, in particular the lips, using a) one or more PHA copolymers as defined previously, b) one or more natural resins as defined previously, optionally c) one or more fatty substances as defined previously, optionally d) one or more organic solvents other than c) and optionally e) water.

More particularly, a subject of the invention is a non-therapeutic cosmetic process for treating keratin materials, comprising the application to the keratin materials of a composition as defined previously. The treatment process is in particular a process for caring for or making up keratin materials.

For the purposes of the present invention and unless otherwise indicated:
the term “cosmetic active agent” means the radical of an organic or organosilicon compound which can be integrated into a cosmetic composition to give an effect on keratin materials, whether this effect is immediate or provided by repeated applications. As examples of cosmetic active agents, mention may be made of coloured or uncoloured, fluorescent or non-fluorescent chromophores such as those derived from optical brighteners, or chromophores derived from UVA and/or UVB screening agents, anti-ageing active agents or active agents intended for providing a benefit to the skin such as active agents having action on the barrier function, deodorant active agents other than mineral particles, antiperspirant active agents other than mineral particles, desquamating active agents, antioxidant active agents, moisturizing active agents, sebum-regulating active agents, active agents intended for limiting the sheen of the skin, active agents intended for combating the effects of pollution, antimicrobial or bactericidal active agents, antidandruff active agents, and fragrances.
the term “(hetero)aryl” means aryl or heteroaryl groups;
the term “(hetero)cycloalkyl” means cycloalkyl or heterocycloalkyl groups;
the “aryl” or “heteroaryl” radicals or the aryl or heteroaryl part of a radical may be substituted with at least one substituent borne by a carbon atom, chosen from:
   a C₁-C₆ and preferably C₁-C₄ alkyl radical;
   a halogen atom such as chlorine, fluorine or bromine;
   a hydroxyl group;
   a C₁-C₂ alkoxy radical; a C₂-C₄ (poly)hydroxyalkoxy radical;
   an amino radical;
   an amino radical substituted with one or two identical or different C₁-C₆ and preferably C₁-C₄ alkyl radicals;
   an acylamino radical (—NR—COR′) in which the radical R is a hydrogen atom;
   a C₁-C₄ alkyl radical and the radical R′ is a C₁-C₄ alkyl radical; a carbamoyl radical ((R)₂N—CO—) in which the radicals R, which may be identical or different, represent a hydrogen atom or a C₁-C₄ alkyl radical;
   an alkylsulfonylamino radical (R′SO₂—NR—) in which the radical R represents a hydrogen atom or a C₁-C₄ alkyl radical and the radical R′ represents a C₁-C₄ alkyl radical, or a phenyl radical;
   an aminosulfonyl radical ((R)₂N—S(O)₂—) in which the radicals R, which may be identical or different, represent a hydrogen atom or a C₁-C₄ alkyl radical;
   a carboxylic radical in acid form or salified (preferably with an alkali metal or a substituted or unsubstituted ammonium) form;
   a cyano group (CN);
   a polyhalo(C₁-C₄)alkyl group, preferentially trifluoromethyl (CF₃);
the cyclic or heterocyclic part of a non-aromatic radical may be substituted with at least one substituent borne by a carbon atom, chosen from the groups:
   hydroxyl,
   C₁-C₄ alkoxy, C₂-C₄ (poly)hydroxyalkoxy;
   alkylcarbonylamino (RCO—NR′—), in which the radical R′ is a hydrogen atom or a C₁-C₄ alkyl radical and the radical R is a C₁-C₂ alkyl radical or an amino radical substituted with one or two identical or different C₁-C₄ alkyl groups;
   alkylcarbonyloxy (RCO—O—), in which the radical R is a C₁-C₄ alkyl radical or an amino radical substituted with one or two identical or different C₁-C₄ alkyl groups;
   alkoxycarbonyl ((RO—CO—) in which the radical R is a C₁-C₄ alkyl radical or an amino radical substituted with one or two identical or different C₁-C₄ alkyl groups;
a cyclic or heterocyclic radical, or a non-aromatic part of an aryl or heteroaryl radical, may also be substituted with one or more oxo groups;
a hydrocarbon-based chain is unsaturated when it includes one or more double bonds and/or one or more triple bonds;
an “aryl” radical represents a monocyclic or fused or non-fused polycyclic hydrocarbon-based group comprising from 6 to 22 carbon atoms, and at least one ring of which is aromatic; preferentially, the aryl radical is a phenyl, biphenyl, naphthyl, indenyl, anthracenyl or tetrahydronaphthyl;
a “heteroaryl” radical represents a monocyclic or fused or non-fused polycyclic, 5- to 22-membered group, comprising from 1 to 6 heteroatoms chosen from nitrogen, oxygen, sulfur and selenium atoms, and at least one ring of which is aromatic; preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridyl, tetrazolyl, dihydrothiazolyl, imidazopyridyl, imidazolyl, indolyl, isoquinolyl, naphthoimidazolyl, naphthooxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridyl, thiazoylimidazolyl, thiopyrylyl, triazolyl and xanthylyl;
a “cyclic” or “cycloalkyl” radical is a monocyclic or fused or non-fused polycyclic, non-aromatic cyclic hydrocarbon-based radical containing from 5 to 22 carbon atoms, which may include one or more unsaturations; the cycloalkyl is preferably a cyclohexyl group;
a “heterocyclic” or “heterocycloalkyl” radical is a monocyclic or fused or non-fused polycyclic 3- to 9-membered non-aromatic cyclic radical, including from 1 to 4 heteroatoms chosen from nitrogen, oxygen, sulfur and selenium atoms; preferably, the heterocycloalkyl is chosen from epoxide, piperazinyl, piperidyl, morpholinyl and dithiolane;
an “alkyl” radical is a linear or branched, in particular C₁-C₆ and preferably C₁-C₄ saturated hydrocarbon-based radical;
an “alkenyl” radical is a linear or branched unsaturated hydrocarbon-based radical comprising one or more conjugated or non-conjugated double bonds;
an “alkynyl” radical is a linear or branched unsaturated hydrocarbon-based radical comprising one or more conjugated or non-conjugated triple bonds;
an “alkoxy” radical is an alkyl-oxy radical for which the alkyl radical is a linear or branched C₁-C₆ and preferentially C₁-C₄ hydrocarbon-based radical;
a “sugar” radical is a monosaccharide or polysaccharide radical, and the 0-protected sugar derivatives thereof such as sugar esters of (C₁-C₆)alkylcarboxylic acids such as acetic acid, sugars containing amine group(s) and (C₁-C₄)alkyl derivatives, such as methyl derivatives, for instance methylglucose. Sugar radicals that may be mentioned include: sucrose, glucose, galactose, ribose, fucose, maltose, fructose, mannose, arabinose, xylose, lactose;
the term “monosaccharide” refers to a monosaccharide sugar comprising at least 5 carbon atoms of formula Cₓ(H₂O)ₓ with x an integer greater than or equal to 5, preferably x is greater than or equal to 6, in particular x is between 5 and 7 inclusive, preferably x=6; they may be of D or L configuration, and of alpha or beta anomer, and also the salts thereof and the solvates thereof such as hydrates;
the term “polysaccharide” refers to a polysaccharide sugar which is a polymer constituted of several saccharides bonded together via 0-oside bonds, said polymers being constituted of monosaccharide units as defined previously, said monosaccharide units comprising at least 5 carbon atoms, preferably 6; in particular, the monosaccharide units are linked together via a 1,4 or 1,6 bond as α (alpha) or β (beta) anomer, it being possible for each oside unit to be of L or D configuration, and also the salts thereof and the solvates thereof such as the hydrates of said monosaccharides; more particularly, they are polymers formed from a certain number of saccharides (or monosaccharides) having the general formula: —[Cₓ(H₂O)_{y})]_{w}— or —[(CH₂O)ₓ]_{w}—, where x is an integer greater than or equal to 5, preferably x is greater than or equal to 6, in particular x is between 5 and 7 inclusive and preferably x=6, and y is an integer which represents x−1, and w is an integer greater than or equal to 2, particularly between 3 and 3000 inclusive, more particularly between 5 and 2500, preferentially between 10 and 2300, particularly between 15 and 1000 inclusive, more particularly between 20 and 500, preferentially between 25 and 200;
the term “sugar bearing amine group(s)” means that the sugar radical is substituted with one or more amino groups NR₁R₂, i.e. at least one of the hydroxyl groups of at least one saccharide unit of the sugar radical is replaced with a group NR₁R₂, with R₁ and R₂, which may be identical or different, representing i) a hydrogen atom, ii) a (C₁-C₆)alkyl group, iii) an aryl group such as phenyl, iv) an aryl(C₁-C₄)alkyl group such as benzyl, vii) —C(Y)—(Y′)ᵣ—R′, with Y and Y′, which may be identical or different, representing an oxygen atom, a sulfur atom or N(R′₂), preferably oxygen, f=0 or 1, preferably 0; and R′₁ and R′₂ representing i) to vi) of R₁ and R₂ defined previously, and in particular R′₁ denoting a (C₁-C₆)alkyl group such as methyl. Preferably, R₁ and/or R₂ represent a hydrogen atom, or a (C₁-C₄)alkylcarbonyl group such as acetyl, and more preferentially R₁ represents a hydrogen atom and R₂ represents a (C₁-C₄)alkylcarbonyl group such as acetyl;
the term “organic or mineral acid salt” more particularly means organic or mineral acid salts in particular chosen from a salt derived from i) hydrochloric acid HCl, ii) hydrobromic acid HBr, iii) sulfuric acid H₂SO₄, iv) alkylsulfonic acids: Alk-S(O)₂OH such as methanesulfonic acid and ethanesulfonic acid; v) arylsulfonic acids: Ar—S(O)₂OH such as benzenesulfonic acid and toluenesulfonic acid; vi) alkoxysulfinic acids: Alk-O—S(O)OH such as methoxysulfinic acid and ethoxysulfinic acid; vii) aryloxysulfinic acids such as tolueneoxysulfinic acid and phenoxysulfinic acid; viii) phosphoric acid H₃PO₄; ix) triflic acid CF₃SO₃H and x) tetrafluoroboric acid HBF₄; xi) organic carboxylic acids R^{o}—C(O)—OH (I′z), in which formula (I′z) R^{o} represents a (hetero)aryl group such as phenyl, (hetero)aryl(C₁-C₄)alkyl group such as benzyl, or (C₁-C₁₀)alkyl, said alkyl group being optionally substituted preferably with one or more hydroxyl groups or amino or carboxyl radicals, R^{o} preferably denoting a (C₁-C₆)alkyl group optionally substituted with 1, 2 or 3 hydroxyl or carboxyl groups; more preferentially, the monocarboxylic acids of formula (I′z) are chosen from acetic acid, glycolic acid, lactic acid, and mixtures thereof, and more particularly from acetic acid and lactic acid; and the polycarboxylic acids are chosen from tartaric acid, succinic acid, fumaric acid, citric acid and mixtures thereof; and xii) amino acids including more carboxylic acid radicals than amino groups, such as γ-carboxyglutamic acid, aspartic acid or glutamic acid, in particular γ-carboxyglutamic acid;
an “anionic counterion” is an anion or an anionic group associated with the cationic charge; more particularly, the anionic counterion is chosen from: i) halides such as chloride or bromide; ii) nitrates; iii) sulfonates, including C₁-C₆ alkylsulfonates: Alk-S(O)₂O⁻ such as methylsulfonate or mesylate and ethylsulfonate; iv) arylsulfonates: Ar—S(O)₂O⁻ such as benzenesulfonate and toluenesulfonate or tosylate; v) citrate; vi) succinate; vii) tartrate; viii) lactate; ix) alkyl sulfates: Alk-O—S(O)O⁻ such as methyl sulfate and ethyl sulfate; x) aryl sulfates: Ar—O—S(O)O⁻ such as benzene sulfate and toluene sulfate; xi) alkoxy sulfates: Alk-O—S(O)₂O⁻ such as methoxy sulfate and ethoxy sulfate; xii) aryloxy sulfates: Ar—O—S(O)₂O⁻; xiii) phosphate; xiv) acetate; xv) triflate; and xvi) borates such as tetrafluoroborate;
the “solvates” represent hydrates and also the combination with linear or branched C₁-C₄ alcohols such as ethanol, isopropanol or n-propanol;
the term “chromophore” means a radical derived from a colourless or coloured compound that is capable of absorbing in the UV and/or visible radiation range at a wavelength λ_{abs} of between 250 and 800 nm. Preferably, the chromophore is coloured, i.e. it absorbs wavelengths in the visible range, i.e. preferably between 400 and 800 nm. Preferably, the chromophores appear coloured to the eye, particularly between 400 and 700 nm (Ullmann's Encyclopedia, 2005, Wiley-VcH, Verlag “Dyes, General Survey”, § 2.1 Basic Principle of Color);
the term “fluorescent chromophore” means a chromophore which is also capable of re-emitting in the visible range at an emission wavelength λₑₘ of between 400 and 800 nm, and higher than the absorption wavelength, preferably with a Stoke's shift, i.e. the difference between the maximum absorption wavelength and the emission wavelength is at least 10 nm. Preferably, fluorescent chromophores are derived from fluorescent dyes that are capable of absorbing in the visible range λ_{abs}, i.e. at a wavelength of between 400 and 800 nm, and of re-emitting in the visible range at a λₑₘ of between 400 and 800 nm. More preferentially, fluorescent chromophores are capable of absorbing at a λ_{abs} of between 420 and 550 nm and of re-emitting in the visible range λₑₘ of between 470 and 600 nm;
the term “optical brightening chromophore” means a chromophore derived from an optical brightening compound or “optical brighteners, optical brightening agents (OBAs)” or “fluorescent brightening agents (FBAs)” or “fluorescent whitening agents (FWAs)”, i.e. agents which absorb UV radiation, i.e. at a wavelength λ_{abs} of between 250 and 350 nm, and of subsequently re-emitting this energy by fluorescence in the visible range at an emission wavelength λₑₘ of between 400 and 600 nm, i.e. wavelengths between blue-violet and blue-green with a maximum in the blue range. Optical brightening chromophores are thus colourless to the eye;
the term “UV-A screening agent” means a chromophore derived from a compound which screens out (or absorbs) UV-A ultraviolet rays at a wavelength of between 320 and 400 nm. A distinction may be made between short UV-A screening agents (which absorb rays at a wavelength of between 320 and 340 nm) and long UV-A screening agents (which absorb rays at a wavelength of between 340 and 400 nm);
the term “UV-B screening agent” means a chromophore derived from a compound which screens out (or absorbs) UV-B ultraviolet rays at a wavelength of between 280 and 320 nm.

Furthermore, unless otherwise indicated, the limits delimiting the extent of a range of values are included in that range of values.

### a) The PHA Copolymer(s)

The composition of the invention comprises as first ingredient a) one or more PHA copolymers comprising at least two different repeating polymer units chosen from the following units (A) and (B), as defined previously. In one variant, a composition can comprise as first ingredient a) one or more PHA copolymers which contain at least two different repeating polymer units (A) as defined previously.

As indicated above, the composition according to the invention comprises as first ingredient a) one or more PHA copolymers which comprise, or preferably consist of, at least two different repeating polymer units chosen from the units (A) and (B) as defined previously.

Preferably, the composition of the invention is a composition, preferably a cosmetic composition, comprising:
a) one or more polyhydroxyalkanoate (PHA) copolymers which contain, and preferably consist of, at least two different repeating polymer units chosen from the units (A) and (B) below, and also the optical or geometrical isomers thereof, the organic or mineral acid or base salts thereof, and the solvates thereof such as hydrates:

—[—O—CH(R¹)—CH₂—C(O)—]— unit (A)

—[—O—CH(R²)—CH₂—C(O)—]— unit (B)

As defined previously; and
b) one or more natural resins; and
c) optionally one or more fatty substances, which are preferably liquid at 25° C. and at atmospheric pressure; and
d) optionally, one or more organic solvents other than c);
e) optionally water;
it being understood that:
   (A) is different from (B) and
the composition contains c) one or more fatty substances and/or e) water; preferably, the composition contains ingredients c)+d)+e).

The term “co-polymer” means that said polymer is derived from the polycondensation of polymeric repeating units that are different from each other, i.e. said polymer is derived from the polycondensation of polymeric repeating units (A) with (B), it being understood that the polymeric units (A) are different from the polymeric units (B), it being possible for said copolymer to be obtained from a single saturated or unsaturated aliphatic carbon source which is optionally substituted and/or interrupted, preferably unsubstituted and uninterrupted, or from several carbon sources, in particular at least one of which is an uninterrupted unsubstituted saturated aliphatic and the other carbon source(s) are saturated or unsaturated aliphatic, optionally substituted notably with a halogen atom such as bromine, or with a cyano group, a Bunte salt, a dithiolane radical, a carboxyl, etc. According to one variant, when a polymer is derived from the polycondensation of polymeric repeating units (A) that are different from each other, the units (A) are different from each other.

According to one embodiment, the copolymer according to the invention is derived from a single carbon source, preferably a single saturated or unsaturated aliphatic carbon source which is optionally substituted and/or interrupted, preferably unsubstituted and uninterrupted.

According to one embodiment, the copolymer according to the invention is derived from several carbon sources, preferably from 2 to 10 carbon sources, more preferentially 2 to 5 carbon sources and even more preferentially 2 carbon sources.

According to one embodiment, the copolymer according to the invention is derived from several carbon sources and at least one is saturated aliphatic. According to a particular embodiment of the invention, the PHA copolymer(s) consist of two different repeating polymer units chosen from the units (A) and (B) as defined previously.

According to a particular embodiment of the invention, the PHA copolymer(s) consist of two different repeating polymer units chosen from the units (A) as defined previously, the units (B) such that R² represents a cyclic or non-cyclic, linear or branched, saturated or unsaturated hydrocarbon-based group comprising from 3 to 30 carbon atoms; in particular chosen from linear or branched (C₃-C₂₈)alkyl and linear or branched (C₃-C₂₈)alkenyl, in particular a linear hydrocarbon-based group, more particularly (C₄-C₂₀)alkyl or (C₄-C₂₀)alkenyl; preferably, the hydrocarbon-based group has a carbon number corresponding to the number of carbon atoms of the radical R¹ from which at least one carbon atom is subtracted, preferably corresponding to the number of carbon atoms of the radical R¹ from which two carbon atoms are subtracted.

More particularly, the PHA copolymer(s) according to the invention comprise the repeating unit of formula (I), and also the optical or geometrical isomers thereof, the organic or mineral acid or base salts thereof, and the solvates thereof such as hydrates:

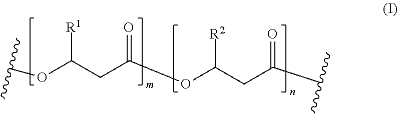

in which formula (I):
   R¹ and R² are as defined previously;
   m and n are integers greater than or equal to 1; preferably, the sum n+m is inclusively between 450 and 1400; preferably, m>n when R¹ and R² represent an unsubstituted and uninterrupted alkyl group—more preferentially, when R¹ and R² are linear alkyl, then R¹ is a C₅-C₁₃ alkyl group; and R² represents a linear alkyl group with a carbon number corresponding to the carbon number of R¹ from which two carbon atoms are subtracted such as a C₃-C₁₁ alkyl group; and
   preferably, m<n when R¹ represents a substituted and/or interrupted alkyl group, an optionally substituted and/or interrupted alkenyl group or an optionally substituted and/or interrupted alkynyl group, and R² represents an alkyl group.

According to a particular embodiment, the PHA copolymer(s) of composition a) contain three different repeating polymer units (A), (B) and (C), and preferably consist of three different polymer units (A), (B) and (C) below, and also the optical or geometrical isomers thereof and the solvates thereof such as hydrates:

—[—O—CH(R¹)—CH₂—C(O)—]— unit (A)

—[—O—CH(R²)—CH₂—C(O)—]— unit (B)

—[—O—CH(R³)—CH₂—C(O)—]— unit (C)
in which polymer units (A), (B) and (C): in which polymer units (A), (B) and (C):
   R¹ and R² are as defined previously;
   R³ represents a saturated or unsaturated, linear or branched, cyclic or non-cyclic, hydrocarbon-based group comprising from 1 to 30 carbon atoms, optionally substituted with one or more atoms or groups a) to m) and/or optionally interrupted with one or more heteroatoms or groups a′) to c′) as defined for R¹; in particular represents a hydrocarbon-based group chosen from linear or branched (C₁-C₂₈)alkyl, and linear or branched (C₂-C₂₈)alkenyl, in particular a linear hydrocarbon-based group, more particularly (C₄-C₂₀)alkenyl; preferably, the hydrocarbon-based group has a carbon number corresponding to the number of carbon atoms in the radical R¹, or else corresponding to the number of carbon atoms in the radical R¹ minus at least three carbon atoms, preferably corresponding to the number of carbon atoms in the radical R¹ minus four carbon atoms; and
it being understood that:
   (A) is different from (B) and (C), (B) is different from (A) and (C), and (C) is different from (A) and (B); and
   preferably, when R¹, R² and R³ represent an unsubstituted and uninterrupted alkyl group, the molar percentage of units (A) is greater than the molar percentage of units (B), and greater than the molar percentage of units (C)—more preferentially, when R¹, R² and R³ are linear alkyl, then R¹ is a C₅-C₁₃ alkyl group; and R² represents a linear alkyl group with a carbon number corresponding to the carbon number of R¹ from which two carbon atoms are subtracted, and R³ represents a linear alkyl group with a carbon number corresponding to the carbon number of R¹ from which four carbon atoms are subtracted; and
   preferably, when R¹ represents a substituted and/or interrupted alkyl, optionally substituted and/or optionally interrupted alkenyl or optionally substituted and/or optionally interrupted alkynyl group, then the molar percentage of units (A) is less than the molar percentage of units (B) and less than the molar percentage of units (C) notably if R² represents an alkyl group and/or R³ represents an alkyl group.

According to a particular embodiment of the invention, the PHA copolymer(s) comprise the repeating unit of formula (II), and also the optical or geometrical isomers thereof, the organic or mineral acid or base salts thereof, and the solvates thereof such as hydrates:

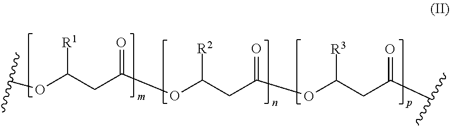

in which formula (II):
   R¹, R² and R³ are as defined previously;
   m, n and p are integers greater than or equal to 1; preferably, the sum n+m+p is inclusively between 450 and 1400; and
   preferably, m>n+p when R¹, R² and R³ represent an unsubstituted and uninterrupted alkyl group—more preferentially, when R¹, R² and R³ are linear alkyl, then R¹ is a C₅-C₁₃ alkyl group; and R² represents a linear alkyl group with a carbon number corresponding to the carbon number of R¹ from which two carbon atoms are subtracted such as a C₃-C₁ alkyl group, and R³ represents a linear alkyl group with a carbon number corresponding to the carbon number of R¹ from which four carbon atoms are subtracted such as a C₁-C₉ alkyl group; and
   preferably, m<n+p when R¹ represents a substituted and/or interrupted alkyl group, an optionally substituted and/or optionally interrupted alkenyl group or an optionally substituted and/or optionally interrupted alkynyl group, and R² and R³ represent an alkyl group.

According to a particular embodiment, the PHA copolymer(s) of composition a) contain four different repeating polymer units (A), (B), (C) and (D), and preferably consist of four different polymer units (A), (B), (C) and (D), below, and also the optical or geometrical isomers thereof, the organic or mineral acid or base salts thereof, and the solvates thereof such as hydrates:

—[—O—CH(R¹)—CH₂—C(O)—]— unit (A)

—[—O—CH(R²)—CH₂—C(O)—]— unit (B)

—[—O—CH(R³)—CH₂—C(O)—]— unit (C)

—[—O—CH(R⁴)—CH₂—C(O)—]— unit (D)
in which polymer units (A), (B), (C) and (D):
   R¹, R² and R³ are as defined previously;
   R⁴ represents a cyclic or non-cyclic, linear or branched, saturated hydrocarbon-based group comprising from 3 to 30 carbon atoms optionally substituted with one or more atoms or groups a) to m) and/or optionally interrupted with one or more heteroatoms or groups a′) to c′) as defined for R¹; it in particular represents a hydrocarbon-based group chosen from linear or branched (C₄-C₂₅)alkyl optionally substituted with one or more atoms or groups a) to m) and/or interrupted with one or more heteroatoms or groups a′) to c′) as defined for R¹; and
it being understood that:
   (A) is different from (B), (C) and (D), (B) is different from (A), (C) and (D), (C) is different from (A), (B) and (D), and (D) is different from (A), (B) and (C); and
   preferably, when R¹, R², R³ and R⁴ represent an unsubstituted and uninterrupted alkyl group, the molar percentage of units (A) is greater than the molar percentage of units (B), greater than the molar percentage of units (C), and greater than the molar percentage of units (D)—more preferentially, when R¹, R², R³ and R⁴ are linear alkyl, then R¹ is a C₅-C₁₃ alkyl group; and R² represents a linear alkyl group with a carbon number corresponding to the carbon number of R¹ from which two carbon atoms are subtracted such as a C₃-C₁ alkyl group, R³ represents a linear alkyl group with a carbon number corresponding to the carbon number of R¹ from which four carbon atoms are subtracted such as a C₁-C₉ alkyl group, and R⁴ represents a linear alkyl group with a carbon number corresponding to the carbon number of R¹ from which six carbon atoms are subtracted; and
   preferably, when R¹ represents a substituted and/or interrupted alkyl, optionally substituted and/or optionally interrupted alkenyl or optionally substituted and/or optionally interrupted alkynyl group, then the molar percentage of units (A) is less than the molar percentage of units (B) and less than the molar percentage of units (C), notably if R² represents an alkyl group and/or R³ represents an alkyl group; and R⁴ represents an optionally substituted and/or optionally interrupted alkyl, optionally substituted and/or optionally interrupted alkenyl or optionally substituted and/or optionally interrupted alkynyl group.

According to a particular embodiment of the invention, the PHA copolymer(s) comprise the repeating unit of formula (III), and also the optical or geometrical isomers thereof, the organic or mineral acid or base salts thereof, and the solvates thereof such as hydrates:

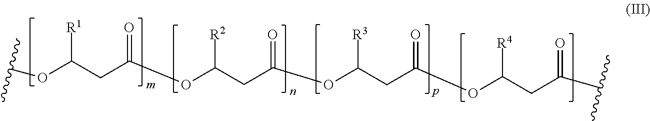

in which formula (III):
   R¹, R², R³ and R⁴ are as defined previously;
   m, n, p and v are integers greater than or equal to 1;
      preferably, the sum n+m+p+v is inclusively between 450 and 1400; and
      preferably, when R¹, R², R³ and R⁴ represent an unsubstituted and uninterrupted alkyl group, then m>n+p+q—more preferentially, when R¹, R², R³ and R⁴ are linear alkyl, then R¹ is a C₅-C₁₃ alkyl group; and R² represents a linear alkyl group with a carbon number corresponding to the carbon number of R¹ from which two carbon atoms are subtracted, R³ represents a linear alkyl group with a carbon number corresponding to the carbon number of R¹ from which four carbon atoms are subtracted, and R⁴ represents a linear alkyl group with a carbon number corresponding to the carbon number of R¹ from which six carbon atoms are subtracted; and
      preferably, when R¹ represents a substituted and/or interrupted alkyl, optionally substituted and/or optionally interrupted alkenyl or optionally substituted and/or optionally interrupted alkynyl group, and R² and R³ represent an alkyl group, and R⁴ represents a substituted and/or interrupted alkyl, optionally substituted and/or optionally interrupted alkenyl or optionally substituted and/or optionally interrupted alkynyl group, then n>m+v; more preferentially n+p>m+v.

According to one embodiment, the PHA copolymer(s) of composition a) more particularly contain five different repeating polymer units (A), (B), (C), (D) and (E), and preferably consist of five different polymer units (A), (B), (C), (D) and (E), below, and also the optical or geometrical isomers thereof, the organic or mineral acid or base salts thereof, and also the solvates thereof such as hydrates:

—[—O—CH(R¹)—CH₂—C(O)—]— unit (A)

—[—O—CH(R²)—CH₂—C(O)—]— unit (B)

—[—O—CH(R³)—CH₂—C(O)—]— unit (C)

—[—O—CH(R⁴)—CH₂—C(O)—]— unit (D)

—[—O—CH(R⁵)—CH₂—C(O)—]— unit (E)
in which polymer units (A), (B), (C), (D) and (E):
   R¹, R², R³ and R⁴ are as defined previously; and
   R⁵ represents a cyclic or non-cyclic, linear or branched, saturated hydrocarbon-based group comprising from 3 to 30 carbon atoms optionally substituted with one or more atoms or groups a) to m) and/or optionally interrupted with one or more heteroatoms or groups a′) to c′) as defined for R¹; it in particular represents a hydrocarbon-based group chosen from linear or branched (C₄-C₂₈)alkyl optionally substituted with one or more atoms or groups a) to m) and/or interrupted with one or more heteroatoms or groups a′) to c′) as defined for R¹; preferably, the hydrocarbon-based group has a carbon number corresponding to the number of carbon atoms of the radical R⁴ from which at least one carbon atom is subtracted, preferably corresponding to the number of carbon atoms of the radical R⁴ from which at least two carbon atoms are subtracted, preferably from which two carbon atoms are subtracted;
it being understood that:
   (A) is different from (B), (C), (D) and (E); (B) is different from (A), (C), (D) and (E); (C) is different from (A), (B), (D) and (E); (D) is different from (A), (B), (C) and (E); and (E) is different from (A), (B), (C) and (D); and
   preferably, when R¹, R², R³, R⁴ and R⁵ represent an unsubstituted and uninterrupted alkyl group, the molar percentage of units (A) is greater than the molar percentage of units (B), greater than the molar percentage of units (C), greater than the molar percentage of units (D) and greater than the molar percentage of units (E)—more preferentially, when R¹, R², R³, R⁴ and R⁵ are linear alkyl, then R¹ is a C₅-C₁₃ alkyl group; and R² represents a linear alkyl group with a carbon number corresponding to the carbon number of R¹ from which two carbon atoms are subtracted, R³ represents a linear alkyl group with a carbon number corresponding to the carbon number of R¹ from which four carbon atoms are subtracted, R⁴ represents a linear alkyl group with a carbon number corresponding to the carbon number of R¹ from which six carbon atoms are subtracted, and R⁵ represents a linear alkyl group with a carbon number corresponding to the carbon number of R¹ from which eight carbon atoms are subtracted; and
   preferably, when R¹ represents a substituted and/or interrupted alkyl, optionally substituted and/or optionally interrupted alkenyl or optionally substituted and/or optionally interrupted alkynyl group, then the molar percentage of units (A) is less than the molar percentage of units (B) and less than the molar percentage of units (C) notably if R² represents an alkyl group and/or R³ represents an alkyl group, and R⁴ and R⁵ represent a substituted and/or interrupted alkyl, optionally substituted and/or optionally interrupted alkenyl or optionally substituted and/or optionally interrupted alkynyl group.

According to a particular embodiment of the invention, the PHA copolymer(s) comprise the repeating unit of formula (IV), and also the optical or geometrical isomers thereof, the organic or mineral acid or base salts thereof, and the solvates thereof such as hydrates:

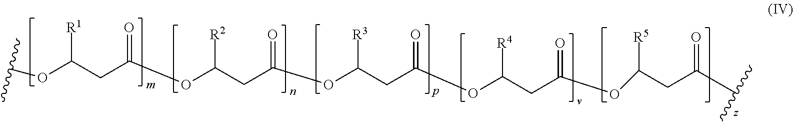

in which formula (IV):
   R¹, R², R³, R⁴ and R⁵ are as defined previously;
   m, n, p, v and z are integers greater than or equal to 1; preferably, the sum n+m+p+v+z is inclusively between 450 and 1400; and
      preferably, when R¹, R², R³, R⁴ and R⁵ represent an unsubstituted and uninterrupted alkyl group, then m>n+p+v+z;
      preferably, when R¹ represents a substituted and/or interrupted alkyl; optionally substituted and/or optionally interrupted alkenyl; or optionally substituted and/or optionally interrupted alkynyl group, R² and R³ represent an alkyl group, and the groups R⁴ and R⁵ represent a substituted and/or interrupted alkyl; optionally substituted and/or optionally interrupted alkenyl; or optionally substituted and/or optionally interrupted alkynyl group, then n>m+v+z; more preferentially n+p>m+v+z.

Preferably, R¹ represents a linear or branched, preferably linear, (C₅-C₂₈)alkyl hydrocarbon-based chain. According to one embodiment of the composition according to the invention, the PHA copolymer(s) are such that the radical R¹ is an alkyl group comprising 5 to 14, preferably from 5 to 12, such as n-pentyl, more preferably between 6 and 12, even more preferably between 6 and 10 carbon atoms, more preferentially between 7 and 10 carbon atoms, better still between 7 and 9 carbon atoms, such as n-hexyl, n-octyl or n-nonyl.

According to a particular embodiment of the invention, the hydrocarbon-based chain R¹ is unsubstituted. According to a particular embodiment of the invention, the hydrocarbon-based chain R¹ is uninterrupted.

According to another embodiment, the hydrocarbon-based chain of the radical R¹ of the invention is 1) either substituted, 2) or interrupted, 3) or substituted and interrupted.

According to a particular embodiment of the invention, the PHA copolymer(s) are such that R¹ represents a hydrocarbon-based chain, notably an alkyl group as defined previously, which is interrupted with one or more (preferably one) atoms or groups chosen from O, S, N(Rₐ) and carbonyl, or combinations thereof such as ester, amide or urea, with Rₐ being as defined previously, preferably Rₐ represents a hydrogen atom; preferably, R¹ represents an alkyl group which is interrupted with one or more atoms chosen from O and S, more preferentially with an O or S, notably S, atom. In particular, when it represents an interrupted hydrocarbon-based chain, notably alkyl, R¹ is C₇-C₂₀, more particularly C₈-C₁₈ and even more particularly C₉-C₁₆. Preferably, said interrupted hydrocarbon-based chain, notably alkyl, is linear.

According to another embodiment of the invention, the PHA copolymer(s) are such that R¹ represents a hydrocarbon-based chain, notably an alkyl group as defined previously, substituted with one or more (preferably one) atoms or groups chosen from: a) to k) as defined previously. Preferably, said hydrocarbon-based chain is substituted with only one atom or group chosen from: a) to k) as defined previously.

According to a particular embodiment of the invention, the PHA copolymer(s) are such that R¹ represents a hydrocarbon-based chain, notably an alkyl group as defined previously, which is substituted with one or more (preferably one) groups chosen from a) halogen such as chlorine or bromine, b) hydroxyl, c) thiol, d) (di)(C₁-C₄)(alkyl)amino and preferably amino, e) carboxyl, i) (hetero)cycloalkyl such as anhydride, dithiolane or epoxide, j) a cosmetic active agent chosen from coloured or uncoloured, fluorescent or non-fluorescent chromophores such as optical brighteners, UV-screening agents, h) (hetero)aryl such as phenyl or furyl, k) R—X with R representing a group chosen from α) cycloalkyl such as cyclohexyl, β) heterocycloalkyl such as a sugar radical, preferably a monosaccharide such as glucosyl, γ) (hetero)aryl such as phenyl, δ) a cosmetic active agent as defined previously, m) thiosulfate and X representing a′) O, S, N(Rₐ), b′) carbonyl, c′) or combinations thereof of a′) with b′) such as ester, amide or urea; Rₐ represents a hydrogen atom or a (C₁-C₄)alkyl or aryl(C₁-C₄)alkyl group such as benzyl, preferably Rₐ represents a hydrogen atom.

Even more preferentially, the PHA copolymer(s) are such that R¹ represents a hydrocarbon-based chain, notably an alkyl group as defined previously, which is substituted with one or more (preferably one) groups chosen from a) halogen such as chlorine or bromine, b) hydroxyl, d) (di)(C₁-C₄)(alkyl)amino, preferably amino, e) carboxyl, i) (hetero)cycloalkyl such as epoxide, h) (hetero)aryl such as phenyl or furyl, k) R—X with R representing a group chosen from α) cycloalkyl such as cyclohexyl, β) heterocycloalkyl such as a sugar radical, preferably a monosaccharide such as glucosyl, γ) (hetero)aryl such as phenyl, and X representing a′) O, S or N(Rₐ), preferably S; Rₐ representing a hydrogen atom or a (C₁-C₄)alkyl group, preferably Rₐ represents a hydrogen atom.

According to one embodiment, said substituted hydrocarbon-based chain, notably alkyl, is linear.

According to another embodiment, said substituted hydrocarbon-based chain, notably alkyl, is branched.

According to another particular embodiment of the invention, the hydrocarbon-based chain of the radical R¹ of the invention is substituted and interrupted.

According to a particular embodiment of the invention, the hydrocarbon-based chain (notably an alkyl group as defined previously) of the radical R¹ of the invention is:
substituted with one or more (preferably one) groups chosen from a) halogen such as chlorine or bromine, b) hydroxyl, c) thiol, d) (di)(C₁-C₄)(alkyl)amino and preferably amino, e) carboxyl, i) (hetero)cycloalkyl such as anhydride, dithiolane or epoxide, j) a cosmetic active agent chosen from coloured or uncoloured, fluorescent or non-fluorescent chromophores such as optical brighteners, UV-screening agents, h) (hetero)aryl such as phenyl or furyl, k) R—X with R representing a group chosen from α) cycloalkyl such as cyclohexyl, β) heterocycloalkyl such as a sugar, preferably a monosaccharide such as glucose, γ) (hetero)aryl such as phenyl, δ) a cosmetic active agent as defined previously and X representing a′) O, S, N(Rₐ), b′) carbonyl, c′) or combinations thereof of a′) with b′) such as ester, amide or urea; Rₐ representing a hydrogen atom or a (C₁-C₄)alkyl or aryl(C₁-C₄)alkyl group such as benzyl, preferably Rₐ represents a hydrogen atom; and
interrupted with one or more (preferably one) atoms or groups chosen from O, S, N(Rₐ) and carbonyl, or combinations thereof such as ester, amide or urea, with Rₐ being as defined previously, preferably Rₐ represents a hydrogen atom; preferably an alkyl group which is interrupted with one or more atoms chosen from O and S, more preferentially with an O or S, notably S, atom. In particular, when it represents an interrupted hydrocarbon-based chain, notably alkyl, the radical R¹ is C₇-C₂₀, more particularly C₈-C₁₈ and even more particularly C₉-C₁₆.

According to a preferred embodiment of the invention, the hydrocarbon-based chain (notably an alkyl group as defined previously) of the radical R¹ of the invention is:
substituted with one or more (preferably one) groups chosen from a) halogen such as chlorine or bromine, b) hydroxyl, d) (di)(C₁-C₄)(alkyl)amino, preferably amino, e) carboxyl, i) (hetero)cycloalkyl such as epoxide, h) (hetero)aryl such as phenyl or furyl, k) R—X with R representing a group chosen from α) cycloalkyl such as cyclohexyl, β) heterocycloalkyl such as a sugar, preferably a monosaccharide such as glucose, γ) (hetero)aryl such as phenyl, and X representing a′) O, S or N(Rₐ), preferably S; Rₐ representing a hydrogen atom or a (C₁-C₄)alkyl group, preferably Rₐ represents a hydrogen atom; and
interrupted with one or more (preferably one) atoms or groups chosen from O, S, N(Rₐ) and carbonyl, or combinations thereof such as ester, amide or urea, with Rₐ being as defined previously, preferably Rₐ represents a hydrogen atom; preferably an alkyl group which is interrupted with one or more atoms chosen from O and S, more preferentially with an O or S, notably S, atom. In particular, when it represents an interrupted hydrocarbon-based chain, notably alkyl, the radical R¹ is C₇-C₂₀, more particularly C₈-C₁₈ and even more particularly C₉-C₁₆.

Preferably, said substituted and interrupted hydrocarbon-based chain is notably alkyl, and is preferably linear.

More preferentially, when said hydrocarbon-based chain R¹ is substituted, it is substituted at the end of the chain on the opposite side from the carbon atom which bears said radical R¹.

According to one embodiment of the invention, said hydrocarbon-based chain R¹ has the following formula —(CH₂)ᵣ—X-(ALK)ᵤ-G with X being as defined previously, in particular representing O, S or N(Rₐ), preferably S, ALK represents a linear or branched, preferably linear, (C₁-C₁₀)alkylene and more particularly (C₁-C₈)alkylene chain, r represents an integer inclusively between 6 and 11, preferably between 7 and 10 such as 8; u is equal to 0 or 1; and G represents a hydrogen atom or a group chosen from hydroxyl, carboxyl, (di)(C₁-C₄)(alkyl)amino, (hetero)aryl in particular aryl such as phenyl, cycloalkyl such as cyclohexyl, or a sugar, in particular a monosaccharide optionally protected with one or more groups such as acyl, preferably Sug.

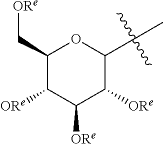

with R^{e} representing a group R^{f}—C(O)—, with R^{f} representing a (C₁-C₄)alkyl group such as methyl; preferably, when u is equal to 0, G represents a cycloalkyl group such as cyclohexyl, or a sugar as defined previously; according to another advantageous variant, when u is equal to 1, G represents a hydrogen atom or a group chosen from hydroxyl, carboxyl, (di)(C₁-C₄)(alkyl)amino or (hetero)aryl, in particular aryl such as phenyl.

According to another particular embodiment of the invention, the PHA copolymer(s) are such that R¹ represents (C₅-C₁₃)alkyl, substituted with a halogen atom such as bromine. Preferably, the halogen atom is substituted at the end of said alkyl group. More preferentially, R¹ represents 1-halo-5-yl such as 1-bromo-5-yl.

According to another particular embodiment of the invention, the PHA copolymer(s) are such that R¹ represents a (C₅-C₁₃)alkyl group, which is preferably linear, substituted with a cyano group g), such as 1-cyano-3-propyl.

According to another particular embodiment of the invention, the PHA copolymer(s) are such that R¹ represents vii) a (hetero)aryl(C₁-C₂)alkyl and more particularly aryl(C₁-C₂)alkyl group, preferably phenylethyl.

According to another particular embodiment of the invention, the PHA copolymer(s) are such that R¹ represents a (C₅-C₂₈)alkyl group substituted with one or more groups chosen from c) (hetero)cycloalkyl. More particularly, R¹ represents a (C₅-C₁₃)alkyl group, which is preferably linear, substituted with a heterocycloalkyl group such as epoxide or dithiolane, preferably epoxide.

In particular, the PHA copolymer(s) are such that R² is chosen from linear or branched (C₃-C₂₀)alkyl or (C₃-C₂₀)alkenyl, preferably linear or branched, and more particularly linear, (C₃-C₂₀)alkyl.

In particular, the PHA copolymer(s) are such that R² is chosen from linear or branched (C₃-C₂₀)alkyl and linear or branched (C₃-C₂₀)alkenyl, in particular a linear hydrocarbon-based group; preferably, the hydrocarbon-based group has a carbon number corresponding to the number of carbon atoms of the radical R¹ from which at least one carbon atom is subtracted, preferably corresponding to the number of carbon atoms of the radical R¹ from which two carbon atoms are subtracted.

According to one embodiment of the invention, the PHA copolymer(s) are such that the radical R² is a linear or branched, preferably linear, (C₃-C₈)alkyl, in particular (C₃-C₆)alkyl, preferably (C₄-C₆)alkyl group such as n-pentyl or n-hexyl.

According to another embodiment of the composition according to the invention, the PHA copolymer(s) comprise a branched (C₃-C₈)alkyl, particularly (C₄-C₆)alkyl radical R², preferably a branched (C₄-C₅)alkyl radical such as isobutyl.

According to another embodiment of the composition according to the invention, the PHA copolymer(s) of the invention comprise the units (A) bearing an alkyl radical R¹ as defined previously, the units (B) as defined previously and the units (C) bearing a linear or branched (C₆-C₂₀)alkenyl, particularly (C₇-C₁₄)alkenyl and more particularly (C₈-C₁₀)alkenyl radical, which is preferably linear and comprising only one unsaturation at the chain end, in particular, —[CR⁴(R⁵)]_{q}—C(R⁶)═C(R⁷)—R⁸ with R⁴, R⁵, R⁶, R⁷ and R⁸, which may be identical or different, representing a hydrogen atom or a (C₁-C₄)alkyl group such as methyl, preferably a hydrogen atom, and q represents an integer inclusively between 2 and 20, preferably between 3 and 10, more preferentially between 4 and 8 such as 6, such as —[CH₂]_{q}—CH═CH₂ and q represents an integer inclusively between 3 and 8, preferably between 4 and 6, such as 5.

According to one embodiment of the composition according to the invention, the PHA copolymer(s) comprise units (A) bearing an alkyl radical R¹ comprising between 8 and 16 carbon atoms substituted with one or more (preferably one) groups chosen from hydroxyl, (di)(C₁-C₄)(alkyl)amino, carboxyl, and R—X—as defined previously, preferably R—S—with R representing a cycloalkyl group such as cyclohexyl, heterocycloalkyl such as a sugar, more preferentially a monosaccharide such as glucose, optionally substituted aryl(C₁-C₄)alkyl such as (C₁-C₄)(alkyl)benzyl or phenylethyl, or heteroaryl(C₁-C₄)alkyl such as furylmethyl.

According to one embodiment of the composition according to the invention, the copolymer(s) comprise units (B) bearing a linear or branched, preferably linear, (C₄-C₅)alkyl radical R² such as pentyl.

According to another embodiment of the composition according to the invention, the PHA copolymer(s) comprise units (A) containing an alkyl radical R¹ as defined previously, units (B) as defined previously and units (C) containing a linear or branched (C₆-C₂₀)alkenyl, particularly (C₇-C₁₄)alkenyl radical and more particularly (C₈-C₁₀)alkenyl radical, which is preferably linear, and comprising only one unsaturation at the chain end such as —[CH₂]_{q}—CH═CH₂ and p represents an integer inclusively between 3 and 8, preferably between 4 and 6, such as 5.

According to a particular embodiment of the invention, in the PHA copolymer(s), the unit (A) comprises a hydrocarbon-based chain as defined previously, in particular ii), said unit (A) preferably being present in a molar percentage ranging from 0.1% to 99%, more preferentially a molar percentage ranging from 0.5% to 50%, even more preferentially a molar percentage ranging from 1% to 40%, better still a molar percentage ranging from 2% to 30%, or a molar percentage ranging from 5% to 20%.

According to a particular embodiment of the invention, in the PHA copolymer(s), the unit (A) is preferably present in a molar percentage ranging from 0.5% to 99%.

According to one embodiment, when R¹ represents a (C₅-C₂₈)alkyl group, the unit (A) is preferably present in a molar percentage ranging from 0.5% to 99%, more preferentially from 50% to 99%, more particularly from 60% to 99% and even more preferentially from 70% to 99%. According to this embodiment, the unit (B) is preferably present in a molar percentage ranging from 0.5% to 40%, preferably from 2% to 40%; and the unit (C) is preferably present in a molar percentage ranging from 0.5% to 20% relative to all the units (A), (B) and (C).

According to another embodiment, when R¹ represents a hydrocarbon-based chain chosen from i) linear or branched (C₅-C₂₈)alkyl, ii) linear or branched (C₅-C₂₈)alkenyl, iii) linear or branched (C₅-C₂₈)alkynyl, preferably the hydrocarbon-based group is linear, said hydrocarbon-based chain being substituted with one or more atoms or groups a) to m) and/or interrupted with one or more heteroatoms or groups a′) to c′) as defined for R¹; it in particular represents a hydrocarbon-based group chosen from linear or branched (C₄-C₂₈)alkyl, optionally substituted with one or more atoms or groups a) to m) and/or interrupted with one or more heteroatoms or groups a′) to c′) as defined previously, the unit (A) is preferably present in a molar percentage ranging from 0.5% to 99%, more preferentially a molar percentage ranging from 1% to 50%, even more preferentially a molar percentage ranging from 5% to 40%, better still a molar percentage ranging from 10% to 30%; the unit (B) is present in a molar percentage ranging from 1% to 99.5%, preferably from 1% to 90%, more preferably from 2% to 70%, or from 2% to 40%; the unit (C) is present in a molar percentage ranging from 0.5% to 20% relative to all the units (A), (B) and (C). Advantageously, the PHA copolymer(s) of the invention comprise from 2 mol % to 70 mol % of units (B), and from 0.5 mol % to 10 mol % of units (C); more advantageously, the copolymer comprises from 5 mol % to 35 mol % of units (B), and from 0.5 mol % to 7 mol % of units (C).

According to a more particular embodiment of the invention, the PHA copolymer(s) are such that, in the PHA copolymer(s) a):
the unit (A) comprises a hydrocarbon-based chain with one or more atoms or groups a) to m) and/or interrupted with one or more heteroatoms or groups a′) to c′) as defined previously, said unit (A) being present in a molar percentage ranging from 0.1% to 99%, preferentially a molar percentage ranging from 0.5% to 50%, more preferentially a molar percentage ranging from 1% to 40%, even more preferentially a molar percentage ranging from 2% to 30%, better still a molar percentage ranging from 5% to 30%, even better still a molar percentage ranging from 10% to 30% of units (A); and
the unit (B) is present in a molar percentage ranging from 2% to 70%, preferentially a molar percentage from 5% to 35% of units (B); and/or
the unit (C) is present in a molar percentage ranging from 0.5% to 20%, preferentially a molar percentage from 0.5% to 10%, more preferentially from 0.5 mol % to 7 mol % of units (C).

Preferably, when R¹ of the unit (A) comprises a saturated unsubstituted and uninterrupted hydrocarbon-based chain, said unit (A) is present in a molar percentage of greater than 30%, more particularly greater than 50%, more preferentially greater than 60%, preferably between 60% and 90%.

The values of the molar percentages of the units (A), (B) and (C) of the PHA copolymer(s) are calculated relative to the total number of moles of (A)+(B) if the copolymer(s) do not comprise any additional units (C); otherwise, if the copolymer(s) of the invention contain three different units (A), (B) and (C), then the molar percentage is calculated relative to the total number of moles (A)+(B)+(C); otherwise, if the copolymer(s) of the invention contain four different units (A), (B), (C) and (D), then the molar percentage is calculated relative to the total number of moles (A)+(B)+(C)+(D); otherwise, if the copolymer(s) of the invention contain five different units (A), (B), (C), (D) and (E), then the molar percentage is calculated relative to the total number of moles (A)+(B)+(C)+(D)+(E).

According to one form of the invention, the PHA copolymer(s) of the invention comprise the following repeating units (A), and also the optical or geometrical isomers thereof, the organic or mineral acid or base salts thereof, and the solvates thereof such as hydrates:

| (A) | R¹ |
|---|---|
| A1 | —ALK₁—S—ALK₂—COOH |
| A2 | —ALK₁—S—ALK₂—H |
| A3 | —ALK₁—S—ALK₂—OH |
| A4 | —ALK₁—S—ALK₂—NH₂ |
| A5 | —ALK₁—S—Cycl′ |
| A6 | —ALK₁—S—CH₂—Fur |
| A7 | —ALK₁—S—Suc |
| A8 | —ALK₁—S—ALK₂—Ar |
| A9 | —ALK₁—Hal |
| A10 | —ALK₁—CN |
| A11 | —ALK₁—CH═CRᵣR_{w} |
| A12 | —ALK₂—H |

in which repeating units A1 to A12:
   ALK₁ represents a divalent linear or branched C₁-C₂₀, preferably linear or branched, more preferentially linear, C₁-C₁₀, hydrocarbon-based radical;
   ALK₂ represents a divalent linear or branched C₁-C₂₀, preferably linear or branched C₁-C₁₂, hydrocarbon-based radical;
   Rr and Rw independently denote a hydrogen atom or a C₁-C₄ alkyl radical such as methyl; preferably, Rr and Rw are identical;
   Hal represents a halogen atom such as bromine;
   Ar: represents a (hetero)aryl group such as phenyl;
   Cycl′: represents a cycloalkyl group such as cyclohexyl or heterocycloalkyl such as dithiolane, or epoxide, preferably epoxide;
   Fur: represents a furyl group, preferably 2-furyl;
   Sug: represents a sugar group, in particular a monosaccharide optionally protected with one or more groups such as acyl, in particular acetyl.

In particular, the stereochemistry of the carbon atoms bearing the radicals R¹ is of (R) configuration.

According to one form of the invention, the PHA copolymer(s) of the invention comprise the repeating units (B) of formula (A12), and also the optical or geometrical isomers thereof, the organic or mineral acid or base salts thereof, and the solvates thereof such as hydrates, it being understood that (B) is different from (A).

Preferentially, the PHA copolymer(s) of the invention comprise the following repeating units, and also the optical or geometrical isomers thereof, the organic or mineral acid or base salts thereof, and the solvates thereof such as hydrates:

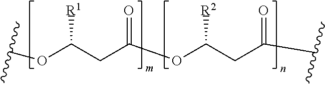

| Compounds | R¹ | R² |
|---|---|---|
| (1) | —(CH₂)₈—S—CH(CH₃)—C(O)—OH | —(CH₂)₄—CH₃ |
| (2) | —(CH₂)₈—S—(CH₂)₇—CH₃ | —(CH₂)₄—CH₃ |
| (3) | —(CH₂)₈—S—(CH₂)₈—OH | —(CH₂)₄—CH₃ |
| (4) | —(CH₂)₈—S—(CH₂)₂—NH₂ | —(CH₂)₄—CH₃ |
| (5) | —(CH₂)₈—S—Cycl | —(CH₂)₄—CH₃ |
| (6) | —(CH₂)₈—S—CH₂-Fur | —(CH₂)₄—CH₃ |
| (7) | —(CH₂)₈—S-Sug | —(CH₂)₄—CH₃ |
| (8) | —(CH₂)₈—S—(CH₂)₂—Ar | —(CH₂)₄—CH₃ |
| (9) | —(CH₂)₈—S—CH₂—Ar′ | —(CH₂)₄—CH₃ |
| (10) | —(CH₂)₈—S—CH(CH₃)—C(O)—OH | —(CH₂)₅—CH₃ |
| (11) | —(CH₂)₅-Hal | —(CH₂)₅—CH₃ |
| (12) | —(CH₂)₃—CN | —(CH₂)₅—CH₃ |
| (13) | 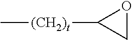 | —(CH₂)₅—CH₃ |
| (14) | —(CH₂)₂—Ar | —(CH₂)₅—CH₃ |
| (15) | —(CH₂)₄—CH₃ | —(CH₂)₂—CH₃ |
| (16) | —(CH₂)₅—CH₃ | —(CH₂)₃—CH₃ |
| (17) | —(CH₂)₆—CH₃ | —(CH₂)₄—CH₃ |
| (18) | —(CH₂)₈—CH₃ | —(CH₂)₆—CH₃ |
| (19) | —(CH₂)₃—CH(CH₃)CH₃ | —CH₂—CH(CH₃)CH₃ |
| (20) | —(CH₂)₆—CH═CH₂ | —(CH₂)₅—CH₃ |
| (21) | —(CH₂)₂—CH═C(CH₃)CH₃ | —CH₂—CH(CH₃)CH₃ |
| (2a) | —(CH₂)₈—S—(CH₂)₇—CH₃ | (CH₂)₅—(CH₃) |

m and n are as defined previously, Hal represents a halogen atom such as bromine and t represents an integer between 1 and 10, preferably between 3 and 8 such as 6.
Ar: represents a (hetero)aryl group such as phenyl;
Ar′: represents a (C₁-C₄)alkyl(hetero)aryl group such as t-butylphenyl, preferably 4-t-butylphenyl;
Cycl: represents a cyclohexyl group;
Fur: represents a furyl group, preferably 2-furyl;
Sug: represents a sugar group, in particular a monosaccharide optionally protected with one or more groups such as acyl; preferably, Sug represents: 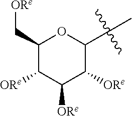
with R^{e} representing a group R^{f}—C(O)—, with R^{f} representing a (C₁-C₄) alkyl group such as methyl.

In particular, the stereochemistry of the carbon atoms bearing the radicals R¹ and R² is of the same (R) or (S) configuration, preferably of (R) configuration.

More particularly, the stereochemistry of the carbon atoms bearing the radicals R¹, R² and R³ is of the same (R) or (S) configuration, preferably of (R) configuration. More particularly, the stereochemistry of the carbon atoms bearing the radicals R¹, R², R³ and R⁴ is of the same (R) or (S) configuration, preferably of (R) configuration.

More particularly, the stereochemistry of the carbon atoms bearing the radicals R¹, R², R³, R⁴ and R⁵ is of the same (R) or (S) configuration, preferably of (R) configuration.

More preferentially, the PHA copolymer(s) have the following formula, and also the optical isomers thereof, the organic or mineral acid or base salts thereof, and the solvates thereof such as hydrates:

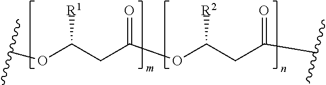

| Compounds | R¹ | R² |
|---|---|---|
| (1′) | —(CH₂)₈—S—CH(CH₃)—C(O)—OH | —(CH₂)₄—CH₃ |
| (2′) | —(CH₂)₈—S—(CH₂)₇—CH₃ | —(CH₂)₄—CH₃ |
| (3′) | —(CH₂)₈—S—(CH₂)₈—OH | —(CH₂)₄—CH₃ |
| (4′) | —(CH₂)₈—S—(CH₂)₂—NH₂ | —(CH₂)₄—CH₃ |
| (5′) | —(CH₂)₈—S—Cycl | —(CH₂)₄—CH₃ |
| (6′) | —(CH₂)₈—S—CH₂-Fur | —(CH₂)₄—CH₃ |
| (7′) | —(CH₂)₈—S-Sug | —(CH₂)₄—CH₃ |
| (8′) | —(CH₂)₈—S—(CH₂)₂—Ar | —(CH₂)₄—CH₃ |
| (9′) | —(CH₂)₈—S—CH₂—Ar′ | —(CH₂)₄—CH₃ |
| (10′) | —(CH₂)₈—S—CH(CH₃)—C(O)—OH | —(CH₂)₅—CH₃ |
| (11′) | —(CH₂)₅-Hal | —(CH₂)₅—CH₃ |
| (12′) | —(CH₂)₃—CN | —(CH₂)₅—CH₃ |
| (13′) | 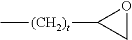 | —(CH₂)₅—CH₃ |
| (14′) | —(CH₂)₂—Ar | —(CH₂)₅—CH₃ |
| (15′) | —(CH₂)₄—CH₃ | —(CH₂)₂—CH₃ |
| (16′) | —(CH₂)₅—CH₃ | —(CH₂)₃—CH₃ |
| (17′) | —(CH₂)₆—CH₃ | —(CH₂)₄—CH₃ |
| (18′) | —(CH₂)₈—CH₃ | —(CH₂)₆—CH₃ |
| (19′) | —(CH₂)₃—CH(CH₃)CH₃ | —CH₂—CH(CH₃)CH₃ |
| (20′) | —(CH₂)₆—CH═CH₂ | —(CH₂)₅—CH₃ |
| (21′) | —(CH₂)₂—CH═C(CH₃)CH₃ | —CH₂—CH(CH₃)CH₃ |
| (22′) | —(CH₂)₄—CH₃ | —(CH₂)₂—CH₃ |
| (23′) | —(CH₂)₅—CH₃ | —(CH₂)₃—CH₃ |
| (24′) | —(CH₂)₆—CH₃ | —(CH₂)₄—CH₃ |
| (2a′) | —(CH₂)₈—S—(CH₂)₇—CH₃ | (CH₂)₅—(CH₃) |

m, n, Hal, t, Ar, Ar′, Cycl, Fur and Sug are as defined previously for compounds (1) to (14).

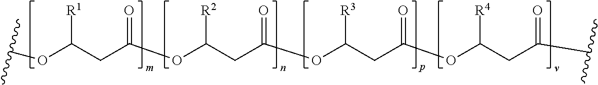

| Compounds | R¹ | R² | R³ | R⁴ |
|---|---|---|---|---|
| (25) | —(CH₂)₈—CH₃ | —(CH₂)₆—CH₃ | —(CH₂)₄—CH₃ | —(CH₂)₂—CH₃ |
| (26) | —(CH₂)₆—CH₃ | —(CH₂)₄—CH₃ | —(CH₂)₂—CH₃ | —CH₃ |
| (27) | —(CH₂)₂—CN | —(CH₂)₅—CH₃ | —(CH₂)₃—CH₃ | —CN |
| (28) | —(CH₂)₂—Ar | —(CH₂)₅—CH₃ | —(CH₂)₃—CH₃ | —Ar |

with m, n, p and v, and Ar as defined previously,

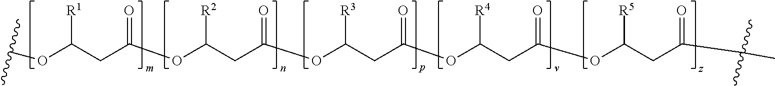

| Com- | | | | | |
|---|---|---|---|---|---|
| pounds | R¹ | R² | R³ | R⁴ | R⁵ |
| (29) | —(CH₂)₉—CH═CH₂ | —(CH₂)₅—CH₃ | —(CH₂)₃—CH₃ | —(CH₂)₇—CH═CH₂ | —(CH₂)₅—CH═CH₂ |
| (30) | —(CH₂)₉—CH═CH₂ | —(CH₂)₄—CH₃ | —(CH₂)₂—CH₃ | —(CH₂)₇—CH═CH₂ | —(CH₂)₅—CH═CH₂ |
| (31) | —(CH₂)₈—CH₃ | —(CH₂)₆—CH₃ | —(CH₂)₄—CH₃ | —(CH₂)₂—CH₃ | —CH₃ |
| (32) | —(CH₂)₁₀—CH₃ | —(CH₂)₈—CH₃ | —(CH₂)₆—CH₃ | —(CH₂)₄—CH₃ | —(CH₂)₂—CH₃ |
| (33) | —(CH₂)₈—S—CH(CH₃)— | —(CH₂)₄—CH₃ | —(CH₂)₂—CH₃ | —(CH₂)₆—S—CH(CH₃)— | —(CH₂)₄—S—CH(CH₃)— |
| | C(O)—OH | | | C(O)—OH | C(O)—OH |
| (34) | —(CH₂)₈—S—(CH₂)₇—CH₃ | —(CH₂)₄—CH₃ | —(CH₂)₂—CH₃ | —(CH₂)₆—S—(CH₂)₇—CH₃ | —(CH₂)₄—S—(CH₂)₇—CH₃ |
| (35) | —(CH₂)₈—S—(CH₂)₈—OH | —(CH₂)₄—CH₃ | —(CH₂)₂—CH₃ | —(CH₂)₆—S—(CH₂)₈—OH | —(CH₂)₄—S—(CH₂)₈—OH |
| (36) | —(CH₂)₈—S—(CH₂)₂—NH₂ | —(CH₂)₄—CH₃ | —(CH₂)₂—CH₃ | —(CH₂)₆—S—(CH₂)₂—NH₂ | —(CH₂)₄—S—(CH₂)₂—NH₂ |
| (37) | —(CH₂)₈—S-Cycl | —(CH₂)₄—CH₃ | —(CH₂)₂—CH₃ | —(CH₂)₆—S-Cycl | —(CH₂)₄—S-Cycl |
| (38) | —(CH₂)₈—S—CH₂-Fur | —(CH₂)₄—CH₃ | —(CH₂)₂—CH₃ | —(CH₂)₆—S—CH₂-Fur | —(CH₂)₄—S—CH₂-Fur |
| (39) | —(CH₂)₈ [symbol] —S-Sug | —(CH₂)₄—CH₃ | —(CH₂)₂—CH₃ | —(CH₂)₆ [symbol] —S-Sug | —(CH₂)₄—S-Sug |
| (40) | —(CH₂)₈—S—(CH₂)₂—Ar | —(CH₂)₄—CH₃ | —(CH₂)₂—CH₃ | —(CH₂)₆—S—(CH₂)₂—Ar | —(CH₂)₄—S—(CH₂)₂—Ar |
| (41) | —(CH₂)₈—S—CH₂—Ar′ | —(CH₂)₄—CH₃ | —(CH₂)₂—CH₃ | —(CH₂)₆—S—CH₂—Ar′ | —(CH₂)₄—S—CH₂—Ar′ |
| (42) | —(CH₂)₈—S—CH(CH₃)— | —(CH₂)₅—CH₃ | —(CH₂)₃—CH₃ | —(CH₂)₆—S—CH(CH₃)— | —(CH₂)₄—S—CH(CH₃)— |
| | C(O)—OH | | | C(O)—OH\| | C(O)—OH |
| (43) | —(CH₂)₅-Hal | —(CH₂)₅—CH₃ | —(CH₂)₃—CH₃ | —(CH₂)₃-Hal | —(CH₂)-Hal |
| (44) | 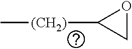 | —(CH₂)₅—CH₃ | —(CH₂)₃—CH₃ | 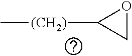 | 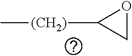 |
| (34a) | —(CH₂)₈—S—(CH₂)₇—CH₃ | —(CH₂)₅—(CH₃) | —(CH₂)₃—(CH₃) | —(CH₂)₆—S—(CH₂)₇—CH₃ | —(CH₂)₄—S—(CH₂)₇—CH₃ |

[symbol]

indicates data missing or illegible when filed

with m, n, p, v and z, Hal, t, Ar, Ar′, Cycl, Fur and Sug being as defined previously.

Preferably, the PHA(s) of the invention are chosen from compounds (15), (16) and (17), notably (16).

More particularly, the PHA(s) of the invention are chosen from compounds (15′), (16′) and (17′), notably (16′).

More particularly, the PHA a) of the invention is compound (23′).

Preferably, the PHA(s) a) of the invention are chosen from compounds (25), (26), (31) and (32), notably (26).

According to a particularly preferred embodiment, the PHA(s) copolymer(s) a) are chosen from the PHA(s) of examples 1″″, 11′, 12, 21 and 25 as described thereafter.

The PHA copolymer(s) of the invention preferably have a number-average molecular weight ranging from 50 000 to 150 000.

The molecular weight may notably be measured by size exclusion chromatography. A method is described below in the examples.

The PHA copolymer(s) are particularly present in composition according to the invention in a content ranging from 0.1% to 65% by weight, preferably from 0.1% to 60%, particularly from 1% to 50% by weight, more particularly from 3% to 40% by weight, more preferably from 5% to 35% by weight, even more preferably from 5% to 30%, better ranging from 5% to 20% by weight relative to the total weight of the composition, or from 10% to 30% or from 15 to 20% by weight relative to the total weight of the composition.

### Method for Preparing the PHA Copolymer(s):

The methods for preparing the PHA copolymer(s) of the invention are known to those skilled in the art. Mention may notably be made of the use of “functionalizable” PHA-producing microbial strains.

The term “functionalizable” means that the PHA copolymer(s) comprise a hydrocarbon-based chain comprising one or more atoms or groups that are capable of reacting chemically with another reagent—also referred to as “reactive atoms or reactive groups”- to give a Z covalent bond with said reagent. The reagent is, for example, a compound comprising at least one nucleophilic group and said functionalized hydrocarbon-based chain comprises at least one electrophilic or nucleofugal atom or group, the nucleophilic group(s) reacting with the electrophilic group(s) to covalently graft Z the reagent. The nucleophilic reagent may also react with one or more unsaturations of the alkenyl group(s) to also lead to grafting by covalent bonding of the functionalized hydrocarbon-based chain with said reagent. The addition reaction may also be radical-based, an addition of Markovnikov or anti-Markovnikov type, or nucleophilic or electrophilic substitution. The addition or condensation reactions may or may not take place via a radical route, with or without the use of catalysts or of enzymes, with heating preferably to a temperature less than or equal to 100° C. or without supplying heat, under a pressure of greater than 1 atm or otherwise, under an inert atmosphere or otherwise, or under oxygen or otherwise.

The term “nucleophilic” refers to any atom or group which is electron-donating by an inductive effect +I and/or a mesomeric effect +M. Electron-donating groups that may be mentioned include hydroxyl, thiol and amino groups.

The term “electrophilic” refers to any atom or group which is electron-withdrawing by an inductive effect −I and/or a mesomeric effect −M.

The microorganisms producing PHAs of the invention notably bearing a hydrocarbon-based chain may be naturally produced by the bacterial kingdom, such as Cyanobacteria of the order of Nostocales (e.g.: Nostoc muscorum, Synechocystis and Synechococcus) but mainly by the Proteobacteria, for example in the class of:
beta-Proteobacteria, of the order Burkholderiales (Cupriavidus negator synonym Ralstonia eutropha)
alpha-Proteobacteria, of the order Rhodobacteriales (Rhodobacter capsulatus marine and photosynthetic)
gamma-Proteobacteria, of the order Pseudomonales of the family Moraxellaceae (Acinetobacter junii).

Among the microorganisms of the bacterial kingdom, the genera Azotobacter, Hydrogenomomas or Chromatium are the most representative of the PHA-producing organisms.

The organisms which naturally produce PHAs bearing notably a C₃-C₅ hydrocarbon-based chain are notably Proteobacteria, such as gamma-Proteobacteria, and more particularly of the order Pseudomonales of the family Pseudomonas such as Pseudomonas resinovorans, Pseudomonas putida, Pseudomonas fluorescens, Pseudomonas aeruginosa, Pseudomonas citronellolis, Pseudomonas mendocina, Pseudomonas chlororaphis and preferably Pseudomonas putida GPo1 and Pseudomonas putida KT2440, preferably Pseudomonas putida and in particular Pseudomonas putida GPo1 and Pseudomonas putida KT2440.

Certain organisms may also naturally produce PHAs without belonging to the order of Pseudomonales, such as Commamonas testosteroni which belongs to the class of beta-Proteobacteria of the order Burkholderiales of the family of Comamonadaceae.

The microorganism producing PHAs according to the invention may also be a recombinant strain if a 3-oxidation PHA synthase metabolic pathway is present. The 3-oxidation PHA synthase metabolic pathway is mainly represented by four classes of enzymes, EC: 2.3.1 B2, EC: 2.3.1 B3, EC: 2.3.1 B4 and EC: 2.3.1 B5.

The recombinant strain may be from the Bacteria kingdom, for instance Escherichia coli, or from the Plantae kingdom, for instance Chlorella pyrenoidosa (International Journal of Biological Macromolecules, 116, 552-562 “Influence of nitrogen on growth, biomass composition, production, and properties of polyhydroxyalkanoates (PHAs) by microalgae”) or from the Fungi kingdom, for instance Saccharomyces cerevisiae or Yarrowia lipolytica: Applied Microbiology and Biotechnology 91, 1327-1340 (2011) “Engineering polyhydroxyalkanoate content and monomer composition in the oleaginous yeast Yarrowia lipolytica by modifying the β-oxidation multifunctional protein”).

Use may also be made of genetically modified microorganisms, which may make it possible, for example, to increase the production of PHA, and/or to increase the oxygen consumption capacity, and/or to reduce the autolysis and/or to modify the monomer ratio.

It is known that, for PHAs, a large portion of the total production cost is devoted to the culture medium and mainly to the substrate/carbon source. Use may thus be made of genetically modified microorganisms using a smaller amount of nutrient (carbon source) for their growth, for example microorganisms that are photo-autotrophic by nature, i.e. using light and CO₂ as main energy source.

The copolymer may be obtained in a known manner by biosynthesis, for example with the microorganisms belonging to the genus Pseudomonas, such as Pseudomonas resinovorans, Pseudomomonas putida, Pseudomonas fluorescens, Pseudomonas aeruginosa, Pseudomonas citronellolis, Pseudomonas mendocina, Pseudomonas chlororaphis and preferably Pseudomonas putida; and with a carbon source which may be a C₂-C₂₀, preferably C₆-C₁₈, carboxylic acid, such as acetic acid, propionic acid, butyric acid, hexanoic acid, heptanoic acid, octanoic acid, nonanoic acid, dodecanoic acid, or an alkenoic acid such as undecylenic acid; a saccharide, such as fructose, maltose, lactose, xylose, arabinose, etc.); an n-alkane, such as hexane, octane or dodecane; an n-alcohol, such as methanol, ethanol, octanol or glycerol; methane or carbon dioxide.

The biosynthesis may optionally be performed in the presence of an inhibitor of the β-oxidation pathway, such as acrylic acid, methacrylic acid, propionic acid, cinnamic acid, salicylic acid, pentenoic acid, 2-butynoic acid, 2-octynoic acid or phenylpropionic acid, and preferably acrylic acid.

According to one embodiment, the process for preparing the PHAs of the invention uses microbial cells which produce PHAs via genetically modified microorganisms (GMOs). The genetic modification may increase the production of PHA, increase the oxygen absorption capacity, increase the resistance to the toxicity of solvents, reduce the autolysis, modify the ratio of the PHA comonomers, and/or any combination thereof. In some of these embodiments, the modification of the comonomer ratio of the unit (A) increases the amount of predominant monomer versus (B) of the PHA of the invention which is obtained. In another embodiment, the PHA-producing microbial cells reproduce naturally.

By way of example, a genetically modified microbial strain producing PHA that is functionalizable or comprising a reactive group that may be mentioned is Pseudomonas entomophila LAC23 (Biomacromolecules. 2014 Jun. 9; 15(6):2310-9. doi: 10.1021/bm500669s).

It is also possible to use genetically modified microorganisms which produce phenylvaleric-co-3-hydroxydodecanoic copolymers (Sci. China Life Sci., Shen R., et al., 57, No. 1, (2014) with a strain such as Pseudomonas entomophila LAC23.

Nutrients, such as water-soluble salts based on nitrogen, phosphorus, sulfur, magnesium, sodium, potassium and iron, may also be used for the biosynthesis.

The appropriate known conditions of temperature, pH and dissolved oxygen (O_{D}) can be used for the culturing of the microorganisms.

The microorganisms may be cultured according to any known method of culturing, such as in a bioreactor in continuous or batch mode, in fed or unfed mode.

The biosynthesis of the polymers used according to the invention is notably described in the article “Biosynthesis and Properties of Medium-Chain-Length Polyhydroxyalkanoates with Enriched Content of the Dominant Monomer”, Xun Juan et al., Biomacromolecules 2012, 13, 2926-2932, and in patent application WO 2011/069244.

The microbial strains producing PHA which is functionalizable or comprising a reactive group, as defined previously, are, for example, of the genus Pseudomonas such as P. cichorii YN2, P. citronellolis, P. jessenii, and more generally with species of Pseudomonas putida such as Pseudomonas putida GPo1 (synonym of Pseudomonas oleovorans), P. putida KT2442, P. putida KT2440, P. putida KCTC 2407 and P. putida BM01, and in particular P. putida KT2440.

The carbon source(s):

One means for gaining access to the PHAs of the invention is to introduce one or more organic compounds into the culture medium, this or these organic compounds representing one or more carbon sources preferably chosen from alkanes, alkenes, alcohols, carboxylic acids and a mixture thereof.

In one embodiment, the organic compound(s) will preferably be chosen from alcohols, carboxylic acids and a mixture thereof.

The carbon source(s) may be classified in two categories:

1) Carbon Source Via One or More Organic Compounds Introduced into the Medium:

According to a particular embodiment of the invention, the organic compound(s) are chosen from alcohols, in particular (C₅-C₂₀)alkanols, and/or carboxylic acids, in particular optionally substituted and/or interrupted (C₅-C₂₀)alkanoic acids, notably (C₅-C₂₀)alkanoic acids such as (C₇-C₁₁)alkanoic acids, for instance nonanoic acid or pelargonic acid and/or (C₅-C₂₀)alkenoic acids, notably (C₅-C₂₀)alkenoic acids such as (C₇-C₁₁)alkenoic acids, for instance undecylenic acid, and mixtures thereof.

The carbon source(s) may be classified into three groups according to their intended use:
group A: the organic compound may aid the growth of the productive strain and aids the production of PHA structural linked to the organic compound.
group B: the organic compound may aid the growth of the strain but does not participate in the production of PHA structurally linked to the organic compound.
group C: the organic compound does not participate in the growth of the strain.

Such microbiological processes are known to those skilled in the art, notably in the scientific literature. Mention may be made of: International Journal of Biological Macromolecules 28, 23-29 (2000); The Journal of Microbiology, 45, No. 2, 87-97, (2007).

According to one variant, the integration of the substrate that is structurally linked to the reactive atom(s) or to the reactive group(s) of the PHA(s) of the invention is introduced directly into the medium as sole carbon source in a medium suitable for microbial growth. (Example: group A for P. putida GPo1: alkenoic acid, notably terminal).

According to another variant, the integration of the substrate that is structurally linked to the reactive atom(s), notably halogen, or to the reactive group(s) of the PHA(s) of the invention is introduced into the medium as carbon source with a second carbon source as co-substrate which is also structurally linked to the PHA, in a medium suitable for microbial growth. (Example: group B for P. putida GPo1: haloalkanoic acids which are preferably terminal, such as terminal bromoalkanoic acids).

According to yet another variant, the integration of the substrate that is structurally linked to the reactive atom(s), notably halogen, or to the reactive group(s) of the PHA(s) of the invention may be introduced directly into the medium as carbon source with a second carbon source as co-substrate which is also structurally linked to the PHA(s) and a third carbon source as co-substrate which is not structurally linked to the PHA(s), in a medium suitable for microbial growth. (Example: group C glucose or sucrose).

In one embodiment, the β-oxidation pathway inhibitor is acrylic acid, 2-butynoic acid, 2-octynoic acid, phenylpropionic acid, propionic acid, trans-cinnamic acid, salicylic acid, methacrylic acid, 4-pentenoic acid or 3-mercaptopropionic acid, preferably acrylic acid.

In one embodiment of the first aspect, the functionalized fatty acid is a functionalized hexanoic acid, functionalized heptanoic acid, functionalized octanoic acid, functionalized nonanoic acid, functionalized decanoic acid, functionalized undecanoic acid, functionalized dodecanoic acid or functionalized tetradecanoic acid.

The functionalization may be introduced by means of an organic compound chosen from precursors of the alcohol and/or carboxylic acid category, notably:
for functionalization of the PHA(s) with a branched alkyl group: see, for example, Applied and Environmental Microbiology, 60, No. 9, 3245-325 (1994);
for functionalization of the PHA(s) with a linear alkyl group comprising a terminal cyclohexyl unit: see, for example doi.org/10.1016/S0141-8130(01)00144-1;
for functionalization of the PHA(s) with an unsaturated alkyl group which is preferably terminal: see, for example, doi.org/10.1021/bm8005616);
for functionalization of the PHA(s) with a linear alkyl group comprising a halogen preferably at the end of the hydrocarbon-based chain (doi.org/10.1021/ma00033a002);
for functionalization of the PHA(s) with a (hetero)aromatic alkyl group, for example phenyl, benzoyl, phenoxy, see, for example, J. Microbiol. Biotechnol., 11, 3, 435-442 (2001);
for functionalization of the PHA(s) with a linear alkyl group comprising a heteroatom notably at the end of the hydrocarbon-based chain, see, for example, DOI 10.1007/s00253-011-3099-4;
for functionalization of the PHA(s) with a linear alkyl group comprising a cyano function notably at the end of the hydrocarbon-based chain, see, for example, doi.org/10.1111/j.1574-6968.1992.tb05839.x;
for functionalization of the PHA(s) with a linear alkyl group comprising an epoxy function notably at the end of the hydrocarbon-based chain, see, for example, doi.org/10.1016/S1381-5148(97)00024-2.

The review International Microbiology 16:1-15 (2013) doi:10.2436/20.1501.01.175 also mentions the majority of the functionalized native PHAs.

In a particular embodiment of the invention, the fatty acid from group A is chosen from 11-undecenoic acid, 10-epoxyundecanoic acid, 5-phenylvaleric acid, citronellol and 5-cyanopentanoic acid.

In a particular embodiment of the invention, the fatty acid from group A is chosen from halooctanoic acids such as 8-bromooctanoic acid.

In a particular embodiment of the invention, the carbon source from group C is a monosaccharide, preferably glucose.

2) Carbon Source in the Presence of Oxidation Inhibitor Introduced into the Medium:

Another aspect of the invention is the use of the PHA-producing microbial strains in a medium that is suitable for microbial growth, said medium comprising: a substrate which is structurally linked to the PHA(s); at least one carbon source which is not structurally linked to the PHA(s); and at least one oxidation and notably β-oxidation pathway inhibitor. This allows the growth of the microbial cells to take place in said medium, the microbial cells synthesizing the PHA polymer(s) of the invention; preferably copolymer particularly containing more than 95% of identical units, which has a comonomer ratio of unit (A) and of unit (B) which differs from that obtained in the absence of the β-oxidation pathway inhibitor.

The scheme below illustrates, by way of example, the functionalization of PHA copolymers according to the invention starting from a PHA copolymer bearing an unsaturated hydrocarbon-based chain, according to Scheme 1 below:

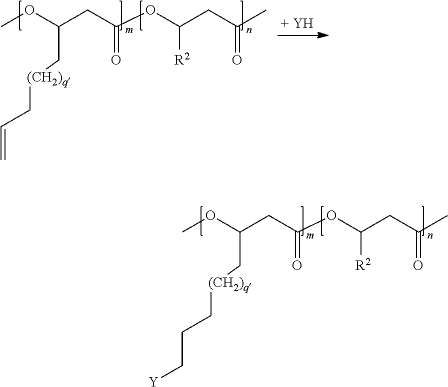

in which Scheme 1:
R², m and n are as defined previously;
Y represents a group chosen from Hal such as chlorine or bromine, hydroxyl, thiol, (di)(C₁-C₄)(alkyl)amino, R—X with R representing a group chosen from α) cycloalkyl such as cyclohexyl, β) heterocycloalkyl such as a sugar, preferably a monosaccharide such as glucose, γ) (hetero)aryl such as phenyl; δ) a cosmetic active agent as defined previously; 6) (C₁-C₂₀)alkyl, (C₂-C₂₀)alkenyl, (C₂-C₂₀)alkynyl; and X representing a′) O, S, N(Rₐ) or Si(R_{b})(Rₑ) or e) linear or branched (C₁-C₂₀)alkyl, with Rₐ, R_{b} and R_{c} as defined previously;
q′ represents an integer inclusively between 2 and 20, preferably between 3 and 10, more preferentially between 4 and 8 such as 6, better still between 3 and 8, preferably between 4 and 6, such as 5.

Other reactions may be performed using double or triple unsaturations such as Michael or Diels-Alder additions, radical reactions, catalytic (notably with Pd or Ni) or non-catalytic hydrogenation reactions, halogenation reactions, notably with bromine, hydration reactions or oxidation reactions, which may or may not be controlled, and reactions on electrophiles as represented schematically below.

According to a particular embodiment of the invention, the PHA copolymers comprise
a linear or branched, saturated hydrocarbon-based chain R¹, substituted and/or interrupted with groups as defined previously for R¹, comprising in total between 5 and 30 carbon atoms, preferably between 6 and 20 carbon atoms and more particularly between 7 and 11 carbon atoms, and
a hydrocarbon-based chain R² representing a linear or branched (C₃-C₂₀)alkenyl, particularly (C₅-C₁₄)alkenyl and more particularly (C₇-C₁₀)alkenyl radical, which is preferably linear and comprising only one unsaturation at the chain end, in particular —[CR⁴(R⁵)]_{q}—C(R⁶)═C(R⁷)—R⁸ with R⁴, R⁵, R⁶, R⁷ and R⁸, which may be identical or different, representing a hydrogen atom or a (C₁-C₄)alkyl group such as methyl, preferably a hydrogen atom, and q represents an integer inclusively between 2 and 20, preferably between 3 and 10, more preferentially between 4 and 8 such as 6, such as —[CH₂]_{q}—CH═CH₂ and q represents an integer inclusively between 3 and 8, preferably between 4 and 6, such as 5,
said chain R² comprising between 1% and 99%, preferentially between 2% and 50% and even more preferentially between 3% and 40% of unsaturations, and even more particularly between 3% and 30% of unsaturations, better still between 5% and 20% of unsaturations. According to this particular embodiment of the invention in which the PHA copolymers comprise unsaturations, these unsaturations may be chemically modified:
A) via addition reactions, such as radical additions, Michael additions, electrophilic additions, Diels-Alder, halogenation, hydration or hydrogenation reaction, and preferably hydrothiolation reaction with particles, chemical compounds or polymers.

In particular, the hydrothiolation reactions may be performed in the presence of a thermal initiator, a redox initiator or a photochemical initiator and of an organic compound bearing a sulfhydryl group, notably chosen from:
linear, branched, cyclic or aromatic alkanethiols including 1 to 14 carbon atoms, such as methane-, ethane-, propane-, pentane-, cyclopentane-, hexane-, cyclohexane-, heptane-, octane-, phenylethane-, 4-tert-butylphenylmethane- or 2-furanmethane-thiol, preferably hexane-, cyclohexane-, heptane-, octane-, phenylethane-, 4-tert-butylphenylmethane- or 2-furanmethane-thiol;
organosiloxanes bearing a thiol function, such as (3-mercaptopropyl)trimethoxysilane, (3-mercaptopropyl)methyldimethoxysilane, 2-(triethoxysilyl)ethanethiol or mercaptopropyl-isobutyl-POSS;
thiolated silicone oils, notably those described in the document DOI: 10.1016/j.actbio.2015.01.020);
thiolated oligomers or polymers bearing a reactive function, such as an amine, an alcohol, an acid, a halogen, a thiol, an epoxide, a nitrile, an isocyanate, a heteroatom, preferably cysteine, cysteamine, N-acetylcysteamine, 2-mercaptoethanol, 1-mercapto-2-propanol, 8-mercapto-1-octanol, thiolactic acid, thioglycolic acid, 3-mercaptopropionic acid, 11-mercaptoundecanoic acid, polyethylene glycol dithiol, 3-mercaptopropionitrile, 1,3-propanedithiol, 4-cyano-1-butanethiol, 3-chloro-1-propanethiol, 1-thio-β-D-glucose tetraacetate; and
thiols which may be obtained from disulfide reduction, such as phenyl disulfide or furfuryl disulfide.

Examples of initiators that may be mentioned include: tert-butyl peroxy-2-ethylhexanoate, cumene perpivalate, tert-butyl peroxylaurate, benzoyl peroxide, lauroyl peroxide, octanoyl peroxide, di-tert-butyl peroxide, tert-butylcumyl peroxide, dicumyl peroxide, 2,2′-azobisisobutyronitrile, 2,2′-azobis(2-methylbutyronitrile), 2,2′-azobis(2,4-dimethylvaleronitrile), 2,2′-azobis(4-methoxy-2,4-dimethylvaleronitrile), 1,1-bis(tert-butylperoxy)-3,3,5-trimethylcyclohexane, 1,1-bis(tert-butylperoxy)cyclohexane, 1,4-bis(tert-butylperoxycarbonyl)cyclohexane, 2,2-bis(tert-butylperoxy)octane, n-butyl 4,4-bis(tert-butylperoxy)valerate, 2,2-bis(tert-butylperoxy)butane, 1,3-bis(tert-butylperoxyisopropyl)benzene, 2,5-dimethyl-2,5-bis(tert-butylperoxy)hexane, 2,5-dimethyl-2,5-bis(benzoylperoxy)hexane, di-tert-butyl diperoxyisophthalate, 2,2-bis(4,4-di-tert-butylperoxycyclohexyl)propane, di-tert-butyl peroxy-α-methylsuccinate, di-tert-butyl peroxydimethylglutarate, di-tert-butyl peroxyhexahydroterephthalate, di-tert-butyl peroxyazelate, 2,5-dimethyl-2,5-bis(tert-butylperoxy)hexane, diethylene glycol bis(tert-butylperoxycarbonate), di-tert-butyl peroxytrimethyladipate, tris(tert-butylperoxy)triazine, vinyltris(tert-butylperoxy)silane phenothiazine, tetracene, perylene, anthracene, 9,10-diphenylanthracene, thioxanthone, benzophenone, acetophenone, xanthone, fluorenone, anthraquinone, 9,10-dimethylanthracene, 2-ethyl-9,10-dimethyloxyanthracene, 2,6-dimethylnaphthalene, 2,5-diphenyl-1,3,4-oxadiazole, xanthopinacol, 1,2-benzanthracene, 9-nitroanthracene. Each of these initiators may be used alone or in combination with others.

The chemical reactions mentioned previously are known to those skilled in the art. Mention may notably be made of the following documents: Synthesis and preparation of PHAs modified with polyethylene glycol dithiol: 10.1021/acs.biomac.9b00479; Biomacromolecules, 19, 3536-3548 (2018); Synthesis and preparation of PHAs modified with mercaptohexanol: 10.1021/acs.biomac.8b01257; Biomacromolecules, 20, 2, 645-652 (2019); Synthesis and preparation of PHAs modified with hydroxycinnamic acid sulfate, and zosteric acid: 10.1021/bm049962e; Biomacromolecules, 5, 4, 1452-1456 (2004); Radical addition of methyl methacrylate to a PHOUn: 10.1002/1521-3935(20010701)202:11<2281::AID-MACP2281>3.0.CO;2-9; Macromolecular Chemistry and Physics, vol. 202, 11, 2281-2286 (2001); Synthesis and preparation of PHAs modified with a polysilsesquioxane (POSS): 10.1016/j.polymer.2005.04.020; Polymer Vol. 46, 14, 5025-5031 (2005); Grafting of thio-beta-glucose onto unsaturated side chains: 1022-1336/99/0202-0091$17.50+0.50/0; Macromol. Rapid Commun., 20, 91-94 (1999); and/or
B) via oxidation reactions, which may or may not be controlled, for example with permanganates of a concentrated or dilute alkaline agent, or ozonolysis, oxidation in the presence of a reducing agent, making it possible to obtain novel materials bearing hydroxyl, epoxide or carboxyl groups in the terminal position of the side chains.

The chemical reactions mentioned previously are known to those skilled in the art. Mention may notably be made of the following documents: 10.1021/bm049337; Biomacromolecules, vol. 6, 2, 891-896 (2005); 10.1016/S0032-3861(99)00347-X; Polymer, vol. 41, 5, 1703-1709 (2000); 10.1021/ma9714528 and 10.1016/S1381-5148(97)00024-2; Macromolecules, 23, 15, 3705-3707 (1990); 10.1016/S0032-3861(01)00692-9; Polymer, vol. 43, 4, 1095-1101 (2002); 10.1016/S0032-3861(99)00347-X; Polymer, vol. 41, 5, 1703-1709 (2000); and 10.1021/bm025728h; Biomacromolecules, vol. 4, 2, 193-195 (2003).

Example of functionalization of PHA copolymers according to the invention starting from a PHA copolymer bearing a hydrocarbon-based chain containing an epoxide group, according to Scheme 2 below:

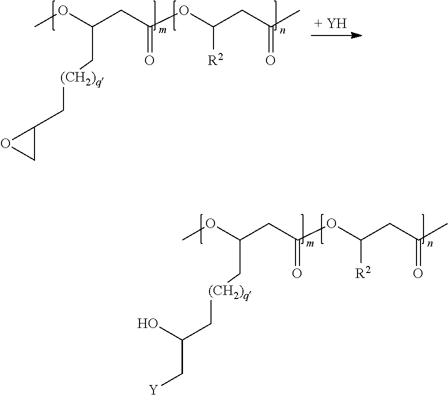

in which Scheme 2 Y, m, n, q′ and R² are as defined in Scheme 1.

The epoxide structure may be obtained via a conventional method known to those skilled in the art, whether via biotechnological processes or via chemical processes such as oxidation of unsaturation as mentioned previously. The peroxide group(s) may react with carboxylic acids, maleic anhydrides, amines, alcohols, thiols or isocyanates, all these reagents including at least one linear or branched, cyclic or acyclic, saturated or unsaturated C₁-C₂₀ hydrocarbon-based chain, or borne by an oligomer or polymer, in particular amino (poly)saccharides such as compounds derived from chitosan and (poly)sil(ox)anes; 3-glycidyloxypropyltrimethoxysilane, 3-aminopropyltriethoxysilane 3-(trimethoxysilyl)propylcarbamic acid, diethanolamine, or 3-mercapto-1-propanesulfonate of alkali metal or alkaline-earth metal salts such as sodium. The epoxide groups may also react with water.

Mention may notably be made of the following documents:
Preparation of PHA bearing charges starting with diethanolamine: 10.1021/bm8005616, Biomacromolecules, vol. 9, 8, 2091-2096 (2008);
Preparation of PHA bearing charges starting with sodium 3-mercapto-1-propanesulfonate: 10.1021/acs.biomac.9b00870 Biomacromolecules, vol. 20, 9, 3324-3332 (2019);
Preparation of PHA including a native epoxide unit: 10.1016/S1381-5148(97)00024-2); Reactive and Functional Polymers, vol. 34, 1, 65-77 (1997).

Example of functionalization of PHA copolymers according to the invention starting from a PHA copolymer bearing a hydrocarbon-based chain containing a nucleofugal group, according to Scheme 3 below:

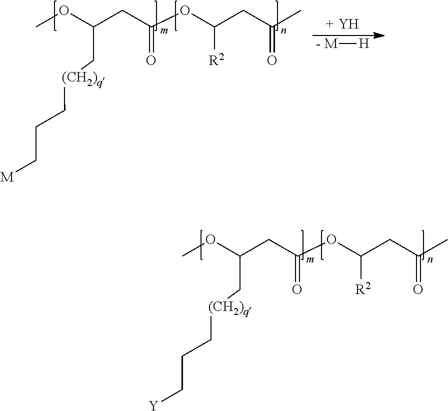

in which Scheme 3 Y, m, n, q′ and R² are as defined in Scheme 1. M corresponds to an organic or inorganic nucleofugal group, which may be substituted with a nucleophilic group; preferably, said nucleophile is a heteroatom which is electron-donating via the +I and/or +M effect such as O, S or N. Preferably, the nucleofugal group M is chosen from halogen atoms such as Br, and mesylate, tosylate or triflate groups. This is a reaction known to those skilled in the art. Mention may be made, for example, of the following document: 10.1016/j.ijbiomac.2016.11.118, International Journal of Biological Macromolecules, vol. 95, 796-808 (2017).

Example of functionalization of PHA copolymers according to the invention starting from a PHA copolymer bearing a hydrocarbon-based chain containing a cyano group, according to Scheme 4 below:

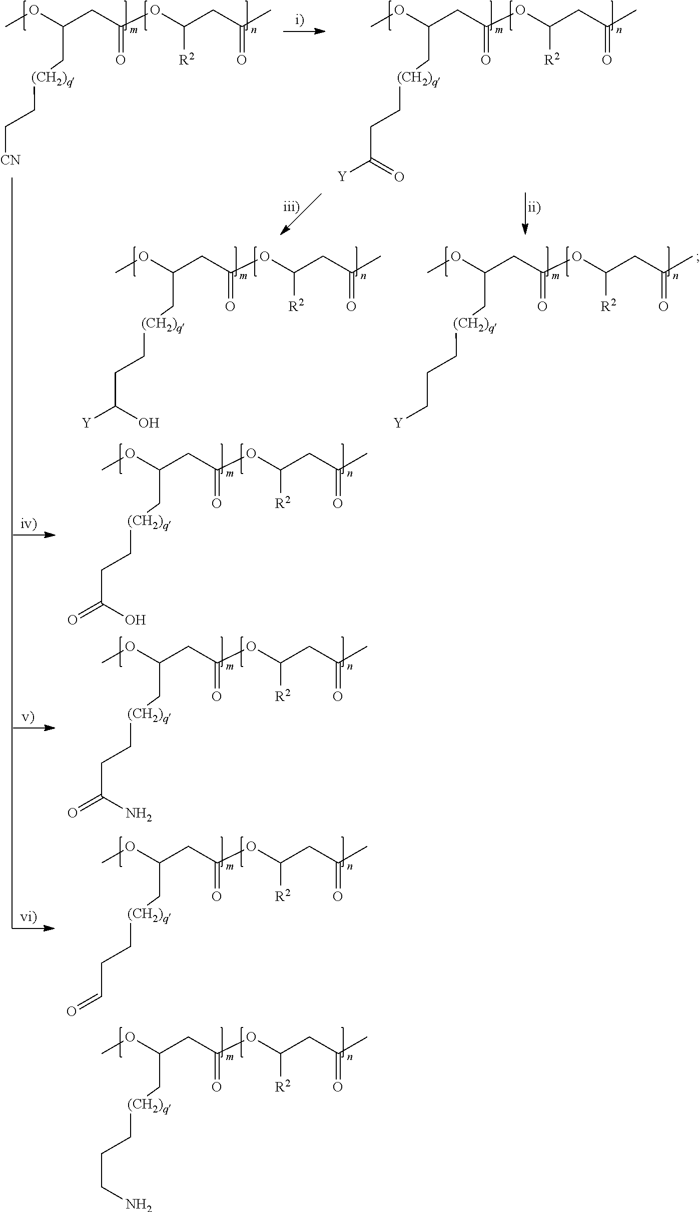

in which Scheme 4 Y, m, n, q′ and R² are as defined in Scheme 1.

In a first step i), the PHA copolymer bearing a side chain containing a cyano or nitrile group reacts with an organo-alkali metal or organomagnesium compound Y-MgHal, Y—Li or Y—Na, followed by hydrolysis to give the PHA copolymer bearing a side chain containing a group Y grafted with a ketone function. The ketone function may be converted into a thio ketone by thionation, for example with S8 in the presence of amine, or with Lawesson's reagent. Said thio ketone, after total reduction ii) (for example by Clemmensen reduction), leads to the PHA copolymer bearing a side chain containing a group Y grafted with an alkylene group. Alternatively, said thio ketone may undergo a controlled reduction iii) with a conventional reducing agent to give the PHA copolymer bearing a side chain containing a group Y grafted with a hydroxyalkylene group. The cyano group of the starting PHA copolymer can react with water after hydration v) to give the amide derivative, after hydrolysis iv) to the carboxyl derivative. The cyano group of the starting PHA copolymer can also, after reduction vi), give the amine derivative or the ketone derivative. PHA copolymers with a hydrocarbon-based chain bearing a nitrile function are prepared via conventional methods known to those skilled in the art. Mention may be made, for example, of the document: 10.1016/0378-1097(92)90311-B, FEMS Microbiology Letters, vol. 103, 2-4, 207-214 (1992).

Example of functionalization of PHA copolymers according to the invention starting from a PHA copolymer bearing a hydrocarbon-based chain at the chain end, according to Scheme 5 below:

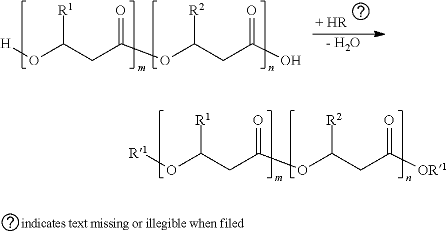

in which Scheme 5 R¹, R², m, n and Y are as defined previously, and R′¹ represents a hydrocarbon-based chain chosen from i) linear or branched (C₁-C₂₀)alkyl, ii) linear or branched (C₂-C₂₀)alkenyl, iii) linear or branched (C₂-C₂₀)alkynyl; preferably, the hydrocarbon-based group is linear; said hydrocarbon-based chain being substituted with one or more atoms or groups chosen from: a) halogens such as chlorine or bromine, b) hydroxyl, c) thiol, d) (di)(C₁-C₄)(alkyl)amino, e) (thio)carboxyl, f) (thio)carboxamide —C(O)—N(Rₐ)₂ or —C(S)—N(Rₐ)₂, f) cyano, g) iso(thio)cyanate, h) (hetero)aryl such as phenyl or furyl, and i) (hetero)cycloalkyl such as anhydride, or epoxide, j) a cosmetic active agent chosen from coloured or uncoloured, fluorescent or non-fluorescent chromophores such as those derived from optical brighteners, or chromophores derived from UVA and/or UVB screening agents, and anti-ageing active agents.

These chain-end grafts onto PHA polymers are known to those skilled in the art. Mention may be made, for example, of the following documents:
Preparation of PHA oligomers by thermal degradation: 10.1021/bm0156274; Biomacromolecules, vol. 3, 1, 219-224 (2002);
Preparation of PHA oligomers by transesterification: 10.1021/ma011420r, Macromolecules, vol. 35, 3, 684-689 (2002);
Preparation of PHA oligomers by hydrolysis: 10.1016/0032-3861(94)90590-8 Polymer vol. 35, 19, 4156-4162 (1994);
Preparation of PHA oligomers by methanolysis: 10.1021/bm060981t, Biomacromolecules, vol. 8, 4, 1255-1265 (2007).

Mention may also be made of other methods known to those skilled in the art:
Synthesis and characterization of PHA grafted with ascorbic acid: 10.1016/j.ijbiomac.2018.11.052; International Journal of Biological Macromolecules, vol. 123: 7 (2019);
Preparation of PHB-b-PHO copolymers by polycondensation with divinyl adipate catalysed with a lipase: 10.1021/bm9011634, Biomacromolecules, vol. 10, 12, 3176-3181 (2009);
Synthesis of PHB-b-PHO copolymers coupled via a diisocyanate junction: 10.1021/ma012223v; Macromolecules, vol. 35, 13, 4946-4950 (2002);
Preparation of PHO oligomers on chitosan by condensation between the carboxylic acid end of the PHO and the amine functions of the chitosan: 10.1002/app.24276; Journal of Applied Polymer Science, vol. 103, 1, (2006);
Transesterification of PHAs with propargyl alcohol in order to produce PHA oligomers that are modifiable by “click” chemistry: 10.1016/j.reactfunctpolym.2011.12.005; Reactive and Functional Polymers, vol. 72, 2, 160-167 (2012);
Preparation of PHO-b-PCL copolymer: 10.1002/mabi.200400104; Macromolecular Bioscience, vol. 4, 11 (2004);
Preparation of PHO-b-PEG copolymer: 10.1002/macp.201000562; Macromolecular Chemistry and Physics; vol. 212, 3, (2010);
Epoxidation of chain-end unsaturation and chain-end grafting of acid: 10.14314/polimery.2017.317; Polimery, vol. 62, 4, 317-322 (2017);
Grafting of organosiloxane unit at chain end onto PHA: 10.1016/j.reactfunctpolym.2014.09.008; Reactive and Functional Polymers, vol. 84, 53-59 (2014).

The combination of grafted PHA copolymers of the invention described previously, according to Scheme 6:

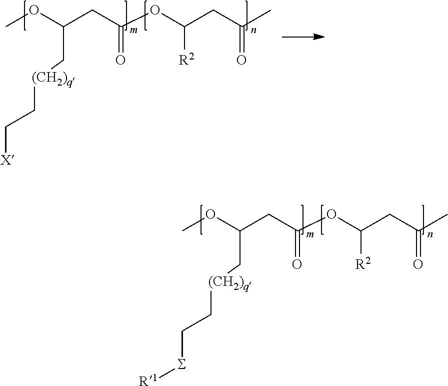

in which Scheme 6 R′¹, R², m, n and Y are as defined previously, and X′ represents a reactive atom or group that is capable of reacting with an electrophilic E or nucleophilic Nu atom or group to create a Σ covalent bond; if X′ is an electrophilic or nucleofugal group, then it can react with a reagent R′¹-Nu; if X′ is a nucleophilic group Nu, then it can react with R′¹-E to create Σ covalent bond.

By way of example, the Z covalent bonds or bonding group that may be generated are listed in the table below, from condensation of electrophiles with nucleophiles:

TABLE 1

| Electropliles [symbol] | Nucleophiles [symbol] | Covalent bonds Σ |
|---|---|---|
| Activated esters* | Amines | Carboxamides |
| Acyl azides** | Amines | Carboxamides |
| Acyl halides | Amines | Carboxamides |
| Acyl halides | Alcohols | Esters |
| Acyl cyanides | Alcohols | Esters |
| Acyl cyanides | Amines | Carboxamides |
| Alkyl halides | Amines | Alkylamines |
| Alkyl halides | Carboxylic acids | Esters |
| Alkyl halides | Thiols | Thioesters |
| Alkyl halides | Alcohols | Ethers |
| Sulfonic acids and salts thereof | Thiols | Thioethers |
| Sulfonic acids and salts thereof | Carboxylic acids | Esters |
| Sulfonic acids and salts thereof | Alcohols | Ethers |
| Anhydrides | Alcohols | Esters |
| Anhydrides | Amines | Carboxamides |
| Aryl halides | Thiols | Thioethers |
| Aryl halides | Amines | Arylamines |
| Aziridines | Thiols | Thioethers |
| Carboxylic acids | Amines | Carboxamides |
| Carboxylic acids | Alcohols | Esters |
| Carbodiimides | Carboxylic acids | N-acylureas |
| Diazoalkanes | Carboxylic acids | Esters |
| Epoxides | Thiols | Thioethers |
| Haloacetamides | Thiols | Thioethers |
| Imide esters | Amines | Amidines |
| Isocyanates | Amines | Ureas |
| Isocyanates | Alcohols | Urethanes |
| Isothiocyanates | Amines | Thioureas |
| Maleimides | Thiols | Thioethers |
| Sulfonic esters | Amines | Alkylamines |
| Sulfonic esters | Thiols | Thioethers |
| Sulfonic esters | Carboxylic acids | Esters |
| Sulfonic esters | Alcohols | Ethers |
| Sulfonyl halides | Amines | Sulfonamides |

*activated esters of general formula —CO-LG with LG representing a leaving group such as oxysuccinimidyl, oxybenzotriazolyl, optionally substituted aryloxy:
**acyl azides can rearrange to give isocyanates

It is also possible, starting with a PHA functionalized on a side chain, to perform chain-end grafting in a second stage as described in Scheme 7. The reverse is also true, in which the chain-end grafting may be performed in a first stage, followed by performing functionalization of a functionalizable side chain in a second stage.

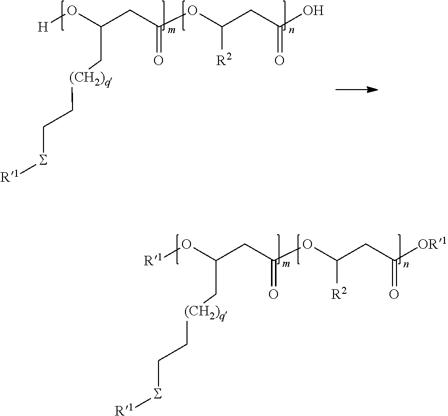

in which Scheme 7 R′¹, R², m, n and Y are as defined previously,

All these chemical reactions are known to those skilled in the art. Mention may be made, for example, of the following documents:
Synthesis and preparation of PHAs modified with thiol-ene followed by reaction on the new grafted function: 10.1021/ma0304426; Macromolecules, vol. 37, 2, 385-389 (2004);
Grafting of PEG and of PLA onto PHAs functionalized with acids: 10.1002/marc.200900803 and 10.1002/mabi.200390033;
Synthesis and preparation of PHAs modified with polyethylene glycol dithiol: 10.1021/acs.biomac.9b00479.

### b) The Natural Resin(s)

The composition of the invention comprises one or more natural resins.

A resin is generally defined as a solid, highly viscous or liquid substance of plant or synthetic origin. Resins have several intrinsic characteristics, such as
the ability to harden permanently, e.g. for synthetic resins under the influence of temperature and for natural resins under the influence of oxygen;
their insolubility in water and above all their good adhesive and tack properties.

The standard ISO4618:2014(fr) defines a resin as being a “generally amorphous macromolecular product with a consistency ranging from solid to liquid”.

Natural resins are almost exclusively of plant origin (fossil or harvested), and are secreted and then exuded from plants for defense, protective and communication purposes within their ecosystems. The exception is shellac of animal origin, secreted by the insect Coccus lacca.

For the purposes of the present invention, the term “natural resin”, and in particular “plant resin”, means any substance comprising a minimum content of terpene compounds, i.e. at least 30% by weight of terpene compounds based on the total weight of the substance (or material) under consideration, as chemically defined hereinbelow, said substance being derived directly or indirectly, from the secretion and exudation, mainly by plants (more rarely by animals), of a substance for defense, protective and communication purposes with their ecosystem.

Advantageously, the natural resin according to the invention is not soluble in water at room temperature (unlike latices or gums, for example).

Natural resins are also considered to be natural adhesives which have the inherent ability to polymerize consistently and predictably on their own without synthetic chemistry.

Preferably, the natural resin used in the composition according to the invention has a number-average molecular weight of less than or equal to 10 000 g/mol. Preferably, the resin has a number-average molecular weight of less than or equal to 10 000 g/mol, notably ranging from 250 to 10 000 g/mol, preferably less than or equal to 5000 g/mol, notably ranging from 250 to 5000 g/mol, better still less than or equal to 2000 g/mol, notably ranging from 250 to 2000 g/mol, and even better still less than or equal to 1000 g/mol, notably ranging from 250 to 1000 g/mol. The number-average molecular weights (Mn) are determined by gel permeation liquid chromatography (THF solvent, calibration curve established with linear polystyrene standards, refractometric detector).

### Thermal Properties

the softening point is the temperature at which a product reaches a certain degree of softening under standardized conditions. It refers to the temperature of transition from a pseudo-solid state to a plastic state upon heating. It may be measured by the ring and ball method (or RBT, ring and ball temperature) for resins according to the standard ASTM E284;
the melting point of a substance at a given pressure is the temperature at which the liquid and solid states of that substance can coexist in equilibrium;
the glass transition temperature (Tg) of a material is the temperature range through which the material changes from a rubbery state to a glassy, solid (rigid) state.

The thermal properties, in particular the m.p. and Tg of the resins may be measured by DSC (Differential Scanning Calorimetry), for example using a Perkin-Elmer DSC 8000 machine, according to:
Protocol 1: Determination of the melting point m.p. and crystallization temperature Tc: the starting materials alone or dissolved/dispersed in solvents, stainless-steel cups, sweep from 5° C. to 90° C., sweep rate at 5° C. min⁻¹.
Protocol 2: Determination of the glass transition temperature Tg: measurement during the 2nd heating. Aluminum cups (40 μL) are used containing the starting materials, a temperature sweep between −100° C. and 150° C. (with isotherms) is performed to observe the glass transition temperature. The temperature ramp applied is 10° C./min for the glass transition temperatures (2 cycles).

Preferably, the resins of the invention have a glass transition temperature, said temperature preferably being in the range from 0 to 200° C., more preferentially from 10° C. to 100° C., even more preferentially from 20° C. to 90° C. and even more preferably from 30° C. to 70° C.

Advantageously, the resins according to the invention are characterized in that they have a softening point, which denotes the temperature of transition from a pseudo-solid state to a plastic state upon heating.

Preferably, the resins of the invention have a softening point (or temperature) in the range from 20° C. to 150° C., more preferentially from 30° C. to 100° C., even more preferentially from 40° C. to 90° C.

Depending on their class, some of the resins according to the invention may also have a melting point, preferably below 360° C., preferentially below 190° C., and even more preferentially below 90° C.

According to a preferred embodiment of the invention, the resins do not have a melting point.

### Botanical Definition of Resins:

More particularly, natural resins are resins of plant or animal origin (ISO 4618/3, and Ullmann's Encyclopedia of Industrial Chemistry, “Resins, Synthetic” 2012 Wiley-VCH Verlag GmbH & Co. KGaA, Weinheim, DOI: 10.1002/14356007.a23_089.pub2).

Natural resins can be classified by their botanical aspects. Resins can be derived from gymnosperms (bare seeders) and angiosperms (covered seeders); the latter are subdivided into monocots (with one leaf embryo) and dicots (with two leaf embryos). They can also be chosen according to their physical and chemical properties.

Natural resins notably comprise rosins (gum, wood or tall oil rosins from tree and plant exudates; extracted woods; or by-products of papermaking), fossil resins such as amber; extracted resins such as asphaltite; shellac such as that produced from insect secretions; and their main derivatives.

Preferably, the resins of the invention are of plant origin, notably from plants or trees.

Fossil resins are resins (hard and semi-hard) collected in the soil at the site of ancient forests which have now disappeared. Some of them are no longer even known with certainty. Some fossil resins have undergone considerable changes in their chemical structure through aging or maturing, which may have taken thousands of years. The transition from fossil to recent resins is variable. They may, for example, comprise resins that are both found fossilized and harvested from living plants. Semi-fossil varieties are collected from the foot of the trees that produced them (Ullmann's Encyclopedia of Industrial Chemistry, “Resins, Natural” 2012 Wiley-VCH Verlag GmbH & Co. KGaA, Weinheim, DOI: 10.1002/14356007.a23_073) (Techniques de l'Ingénieur, “Résines Naturelles”, 1982 Bernard Delmond).

Harvested resins are recent (soft). They are harvested from plants that are all alive. Depending on their composition, they are subdivided into:
oleoresin: natural solution of resin in an essential oil;
balsam: resin characterized by a large proportion of benzoic and cinnamic acids and esters thereof;
gum: resin composed essentially of polysaccharides;
gum-resin: mixture of resins and hydrophilic gums;
   latex: milky composition of organic materials dispersed in an aqueous medium (Techniques de l'Ingénieur, “Résines Naturelles”, 1982 Bernard Delmond)

Among the resins, in particular the recent resins, of the invention, resins that are soluble in oils and/or alcohols are preferred relative to the water-soluble forms such as latices or gums.

According to a preferred embodiment of the invention, the resins of the invention are harvest resins; they are particularly advantageous according to the invention from an ecological viewpoint since they are self-regenerating.

Preferably, the resins of the invention are recent. Advantageously, the resins used according to the invention are based on resources which are not in competition with those intended for food applications. Advantageously, the resins used in the compositions of the invention originate from the upgrading of co-products of the paper industry.

### Chemical Definition of Resins

Chemically, natural resins are complex mixtures of several classes of compounds whose presence and content define the class of the resin (oleoresin, balsam, gum, etc.): essential oils, neutral and acidic constituents and polysaccharides (present exclusively in gums).

The characteristic components of resins are the terpene compounds they contain, preferably in a content of at least 30% by weight, relative to the weight of the resin.

The term “terpene compounds” means terpenes, hydrocarbons formed from isoprene of general formula (C₅H₈)₁, and their numerous derivatives (alcohols, aldehydes, ketones, acids, etc.) comprising a terpene structure (Montpellier Academy. Resins. https://tice.ac-montpellier.fr/ABCDORGA/Famille/Terpenes.html).

Among the terpene hydrocarbons, the following are distinguished: monoterpenes of empirical formula C₁₀H₁₆ (n=2), sesquiterpenes of empirical formula C₁₅H₂₄ (n=3), diterpenes (C₂₀H₃₂) (n=4), sesterterpenes (C₂₅H₄₀) (n=5), triterpenes (C₃₀H₄₈) (n=6), tetraterpenes (C₄₀H₆₄) (n=8) and other polyterpenes. Some have an acyclic structure; they have a number of double bonds corresponding to their empirical formula: 3 for C₁₀H₁₆; 5 for C₂₀H₃₂; 7 for C₃₀H₄₈. Others have one or more rings, i.e. a smaller number of double bonds; for example, for C₁₀H₁₆ one ring and two double bonds or two rings and one double bond.

Advantageously, the resins of the invention contain at least 30% of terpene compounds, preferably at least 40% by weight of terpene compounds, preferably at least 50% terpene compounds, and even more preferably at least 60% of terpene compounds, by weight relative to the total weight of resin or resinous substance used as starting material in the composition according to the invention.

Monoterpene and sesquiterpene compounds are mostly volatile compounds, constituting for example essential oils. Polyterpene compounds derived from terpenes with n greater than or equal to 4 (such as diterpenes and triterpenes derivatives) are resinous compounds of a rather solid nature.

According to a preferred embodiment of the invention, the resins comprise at least 10%, preferably at least 20% by weight, preferably at least 30% by weight, preferably at least 35% by weight, of polyterpene compounds, i.e. derived from terpenes with n greater than or equal to 4, relative to the total weight of the resin representing 100%. Resins with a solid fraction at room temperature (25° C.) are thus preferred. Advantageously, said resins used according to the invention are not volatile.

Advantageously, the polyterpene compounds of the resins or resinous substances used in the composition of the invention are predominantly (to more than 50% by weight relative to the total weight of polyterpenes) derived from diterpenes and/or triterpenes.

According to a preferred embodiment of the invention, the resins comprise less than 70% by weight of monoterpene or sesquiterpene compounds, i.e. derived from terpenes with n less than 4, relative to the total weight of the resin representing 100%; preferably, said resins comprise less than 60% by weight, preferably less than 50% by weight, preferably less than 30% by weight, preferably less than 15% by weight, of monoterpene or sesquiterpene compounds, derived from terpenes with n less than 4, relative to the total weight of the resin representing 100%. It is thus preferred, for the compositions of the invention, to limit the use of the most volatile resins, since they are less effective in terms of cosmetic film persistence.

A non-exhaustive list of the terpene compounds that may be contained in the natural resins of the invention has been compiled. It lists families of terpene compounds, subdivided according to the characteristic groups (alcohol function, ketone function, acid function, etc.) of each compound (Tables Lists below).

The amount of terpene or generally of ingredients contained in the resins is defined by the conventional spectroscopic and spectrometric methods known to those skilled in the art, for example:
such as gas chromatographic analysis GC, chromatographic analysis with flame ionization detection named GC-FID, or GC/MS analysis which consists of the use of a mass spectrometer coupled to a gas chromatograph; preferably by GC-FID (see, for example, GC (gas chromatography) and GC/MS (gas chromatography coupled to mass spectrometry): “Methodological developments in TLC/MALDITOF MS and GC/MS for the analysis of terpenoid compounds present in plant resins” https://tel.archives-ouvertes.fr/tel-01581308.

### Examples of Monoterpene Compounds

Advantageously, the monoterpene compound(s) of the resin are chosen from: α-pinene, β-pinene, 3-carene, camphene, dipentene, p-cymene, β-myrcene, α-phellandrene, sabinene, α-thuyene, limonene, octyl ethanoate, neryl ethanoate, bornyl ethanoate, geranyl ethanoate, α-terpineol, cineol, linalool, borneol, derivatives thereof, and mixtures thereof.

### Examples of Sesquiterpene Compounds

Advantageously, the sesquiterpene compound(s) of the resin are chosen from: α-copaene, β-caryophyllene, β-bisabolene, β-gurjunene, α-gurjunene, allo-aromadendrene, β-bourbonene, δ-cadinene, α-guaiene, α-elemene, β-elemene, δ-elemene, α-copaene, α-selinene, β-selinene, β-bourbonene, lindestrene, furanoeudesma-1,3-diene, α-cubebene, farnesol, α-elemol, viridiflorol, t-cadinol, β-elemol, germacrone, curzerenone, derivatives thereof, and mixtures thereof.

### Examples of Diterpene Compounds

Advantageously, the diterpene compound(s) of the resin are chosen from: abietic acid, pimaric acid, sandarcopimaric acid, comunic acid, levopimaric acid, palustric acid, isopimaric acid, dehydroabietic acid, neoabietic acid, agathic acid, cembrene A, cembrene C, isocembrene, vercilla-4(20),7,11-triene, incensole, totarol, sandaracopimarinol, cembrenol, derivatives thereof, and mixtures thereof.

### Examples of Triterpene Compounds

Advantageously, the triterpene compound(s) of the resin are chosen from:
3β,20(S)-dihydroxydammar-24-ene, dammarenolic acid, dammardienone, hydroxydammarenone (i or ii), dammarenediol i (or ii), dammadienol, 11-keto-β-boswellic acid (kba), 11-keto-β-boswellic acid acetate (akba), β-boswellic acid, ursolic acid, mangiferonic acid, benthamic acid, ursolic aldehyde, α-amyrenone, α-amyrin, β-amyrin, uvaol, oleanolic acid, oleanonic acid, moronic acid, oleanonic aldehyde, acetyl lupeolic acid, lupeolic acid, lupeol,
betulonal, hydroxyhopanone, derivatives thereof, and mixtures thereof.

According to one embodiment of the invention, the resin(s) used according to the present invention preferably contain at least one diterpene compound, preferably derived from abietic acid, natural or chemically modified.

Preferably, the diterpene compound(s), notably derived from abietic acid, are present in the resin in weight contents of at least 20%, preferably at least 30%, and even more preferentially at least 40%, by weight relative to the total weight of the natural resin.

Mention may notably be made of colophony resins t) such as rosinates, containing such diterpene compounds.

According to another embodiment of the invention, the resin(s) used according to the present invention preferably contain at least one triterpene compound, preferably chosen from the following triterpene compounds: α-amyrin, β-amyrin, α-amyrone, β-amyrone, dammadienone, dammadienol, ursolic aldehyde, hydroxyhopanone, oleanonic aldehyde, oleanolic acid, oleanonic acid, oleanolic acid, and mixtures thereof.

The total content of triterpene compounds, notably the content of those preferred above, in the resin used according to the invention is advantageously at least 10%, preferably at least 20%, even more preferentially at least 30%, and preferably at least 35% by weight relative to the total weight of the natural resin.

Mention may notably be made of resins from frankincense k) Protium heptaphyllum or Shorea robusta, containing such triterpene compounds.

Definition of Resins by their Origin:

Mention may notably be made of resins from frankincense k) Protium heptaphyllum or even Shorea robusta, containing such triterpene compounds.

Advantageously, the natural resin(s) according to the invention are chosen from: a) acaroid resins, b) ambers, c) asphaltite and gilsonite, d) Peruvian balsam, e) Tolu balsam, f) benzoin resins, g) Canadian balsam, h) copal resins (notably Kauri copal resins, Manila copals, West African copals such as Congo, Angola or Cameroon copals, East African copals such as Zanzibar or Madagascar copals, South American copals such as Brazilian or Colombian copals), i) dammar resins, j) elemi resins, k) frankincenses, l) galbanums, m) labdanums, n) mastics, o) myrrh, p) sandarac, q) shellacs, r) Styrax (storax), s) Venice turpentine (larch, essence of turpentine), t) colophonies, notably rosin and rosinate, and tall oils, v) resins extracted from plant waxes; and mixtures of these resins.

Preferably, the natural resin(s) used according to the invention are chosen from j), k), t) and v); it is understood that the resin(s) of the invention may be esterified, salified, adducted, modified with phenols, and/or dimerized and also hydrogenated.

### a) Acaroid Resins

Acaroid resin is a recent resin isolated from Xanthorrhoea species, produced in Australia. Appearance. Resins of known acaroid types are yellow or red and differ from each other also in their physical properties. The red acaroid resin is the most important. Acid number 60-110 (red), 125-140 (yellow); saponification number 160-200 (red), 200-240 (yellow). In contrast to the red resin type, the yellow type contains cinnamic and benzoic acids, p-coumaric acid esters, and xanthoresinotannol (about 80%). The red type contains erythroresinotannol (about 85%).

According to one embodiment, the natural resin(s) are chosen from: a) acaroid resins.

### b) Ambers

Ambers are fossil resins that originate from conifers of the Tertiary period (Pinites succinifera) and were probably formed by successive flows of resins, molded into their present forms by pressure, temperature and other influences over thousands of years. Ambers are found mainly in Sambia (Baltic coast) and the Kaliningrad Peninsula (Palmnicken region). They are obtained either by collecting or fishing on the coast (sea amber) or by isolating “blue earth”, which is mined in Palmnicken. Ambers range from bright yellow to brownish (amber-coloured). The resin has an angular or rounded shape of various sizes and may contain inclusions. Amber is a very hard resin that is practically insoluble in all solvents and is a very good dielectric. Amber acquires a negative charge through mechanical friction and was therefore called “electronin” from ancient Greek. It decomposes at about 370° C. without melting. Unlike other resins, amber contains organically bound sulfur. The composition of amber can vary considerably. The approximate elemental composition can be found in the literature: 78% carbon, 9.9% hydrogen, 11.7% oxygen, 0.4% sulfur, and 0.2% ash; acid number 15-35, saponification number 90-125.

According to a particular embodiment of the invention, the resin(s) are chosen from b) ambers.

### c) Asphaltite and Gilsonite

Asphaltite is a fossil resin (cf. asphalt and bitumen, Chap. 2.) which is also known as “glossy pitch”. It is a neutral resin based on thermoplastic hydrocarbons. According to DIN 55 946, asphaltite is defined as a natural asphalt with a low mineral content and high hardness. This natural product appears to be formed from crude oil by evaporation of volatile components and by natural auto-oxidation reaction, polymerisation. Natural hard asphalt (asphaltite) is found in Utah/Colorado (gilsonite), Virginia, and Syria. The Utah asphalt deposit was discovered in 1860 by S. H. Gilson. Asphalts are mined from Trinidad (Trinidad asphalt), Bermuda, Cuba, Mexico and Palestine. Gilsonite is dark brown to black in colour, and shiny.

Gilsonite has a softening point of 145-195° C. (ring and ball); an acid number of 2.3; and a density of 1.03 to 1.15 g/cm³. It is soluble in carbon sulfide and aromatics, less soluble in aliphatic and mineral oils. Gilsonite thickens readily in solution on storage, lower alcohols and glycols act as diluents; gilsonite is only partially soluble in ketones and some glycol ethers.

The approximate elemental composition of gilsonite is 80% to 90% by weight (such as 85%) of carbon, between 5% and 15% by weight (such as 10%) of hydrogen, between 1% and 5% by weight (such as 2.5%) of nitrogen, between 0.5% and 2.5% by weight (such as 1.5%) of oxygen and between 0.1% and 1% by weight (such as 0.3%) of sulfur. Gilsonite contains 40-70% by weight (such as 50-65%) of asphaltenes, 30-50% by weight (such as 35-45%) of resins, 0.5-10% by weight (such as 1-5%) of oils and 0.05-0.5% by weight (such as 0.2%) of ash. As the softening point increases, the proportion of asphaltenes increases and resins and oils decrease. According to a particular embodiment of the invention, the resin(s) are chosen from Trinidad asphalt. Said asphalt particularly contains between 75% and 90% by weight (such as 82%) of carbon, between 5% and 15% by weight (such as 10%) of hydrogen, between 0.5% and 2% by weight (such as 1%) of nitrogen, between 3% and 10% by weight (such as 6%) of sulfur, and between 0.1% and 1% by weight (such as 0.5%) of oxygen.

According to a particular embodiment of the invention, the resin(s) are chosen from c) asphaltite and gilsonite.

### d) Peruvian Balsam

Peruvian balsam is obtained from Myroxylon balsamum (Toluifera pereirae Baill.) trees occurring in Central and South America. Peruvian balsam is a viscous brown-yellow liquid. Peruvian balsam contains large amounts of benzyl cinnamate and benzoate. Its acid number is 60-80; saponification number 230-255. Peruvian balsam is used in perfumes, cosmetics and medicines (for treating eczema).

According to a particular embodiment of the invention, the resin(s) are chosen from d) Peruvian balsam.

### e) Tolu Balsam

Tolu balsam is a recent resin which comes from South America (Colombia, Peru and Venezuela). It is extracted from the living trunks of Myroxylon toluiferum trees. Tolu balsam is brownish, sticky, semi-solid, slowly becoming solid and brittle. Its acid number is 112-168; and its saponification number is 154-216. Tolu balsam is soluble in ether, ethanol and alkalis. In addition to the resin, the balsam contains a large proportion of benzoic and cinnamic acids, esters thereof, and vanillin.

According to a particular embodiment of the invention, the resin(s) are chosen from e) Tolu balsam.

### f) Benzoin Resins

Benzoin resins are recent resins; they are formed only after making a wound on the tree. They are pathological resins. They are produced with various botanical species of styraceae such as Styrax tonkinensis which are notably found in Thailand, Malaysia and Indonesia. More particularly, benzoin resins are chosen from (1) fairly regularly shaped, rounded or flat, light brown beads (their odour is similar to vanilla); and (2) more irregularly shaped yellow, orange, brown to reddish crystals (impure). Its odour is balsamic, resinous and spicy, slightly vanilla-like. There are differences between Siamese benzoin, Sumatran benzoin and Penang benzoin resin. Sumatran benzoin resin contains cinnamic acid in addition to benzoic acid. Sumatran benzoin resin is soluble in alcohol to about 75% by weight. Siamese benzoin resin is up to 90% soluble.

According to a particular embodiment of the invention, the resin(s) are chosen from f) benzoin resins.

### g) Canada Balsam

Canada balsam is a recent resin which comes from Abies balsamea (balsam fir, Abies canadiensis), which is found in Canada and the United States. It is extracted by tapping fir trees by making cuts under the bark. Canada balsam is a liquid that is colourless to greenish, viscous, and slowly solidifying.

Properties. Canada balsam has an acid number of 82-87 and a saponification number of 89-100; density 0.985-0.995 g/cm³; refractive index 1.5180-1.5210 (similar to glass); highly soluble in ethanol. Canada balsam belongs to the terpene hydrocarbons.

According to a particular embodiment of the invention, the resin(s) are chosen from g) Canada balsam.

### h) Copal Resins or Copals

Copals is the generic term to define recent natural fossil resins with different structures, qualities and botanicals according to their geographical origins: Kauri copal resins, Manila copal, West African copals (e.g. Congo, Angola or Cameroon copals), East African copals (e.g. Zanzibar or Madagascar copal), South American copals (e.g. Brazilian or Colombian copal).

The most important types are the Kauri, Manila and Congo copals; the other types are described in H. E. Scheiber: Zerbe, Mineral öle und verwandte Produkte, 2nd ed, Springer Verlag, Berlin 1969, page 698. J. Scheiber: Lacke und ihre Rohstoffe, Verlag Johann Ambrosius Barth, Leipzig 1926. Most types of copal are sparingly soluble in common solvents and sparingly compatible in oils.

### h1) Kauri Copal Resin.

Different types of Kauri copal resin exist. They all come from the Kauri spruce (Agathis australis) grown in New Zealand.

The resins are either extracted (fossil), or are dug from the topsoil (bush copal, recent—fossil), or harvested from living trees (copal tree, recent). Kauri copal is whitish yellow, but can also be dark; the surface is patinated and opaque.

The acid number is 50-100; the saponification number is 75-120; and the softening point is 110-115° C. (Kraemer Sarnow). Kauri copal is completely soluble in higher alcohols (C number >4), moderately soluble in lower alcohols (C≤4) and sparingly soluble in aromatic and aliphatic solvents.

Manila copal resin is a recent fossil resin, which is isolated from Dammara orientalis, which is found in the Philippines and Indonesia.

Manila copal is harvested from living trees. A soft, alcohol-soluble resin is obtained, which is insoluble in aromatic solvents and aliphatic solvents. Manila copal (loba) resin is semi-hard and insoluble in ethanol. Manila copal can be melted, resulting in losses of up to 20%. It is a yellowish, often reddish to dark resin. The main component is agathene dicarboxylic acid (C₂₀H₃₀O₄), derived from diterpenes, which is also present in Kauri copal.

According to a particular embodiment of the invention, the resin(s) are chosen from the copal resins h.

### i) Dammars

Dammars are recent fossil resins which are obtained from trees of the family Dipterocarpaceae. Preferably, the dammars of the invention are chosen from Batavia dammar, and Thailand dammar. The latter are white. According to another embodiment, the dammars of the invention are chosen from the dammars of Eastern India and Sumatra. These dammars are brown and black.

White dammars are irregularly shaped, with transparent yellowish-white or reddish pieces of different sizes. Generally they have a composition with: between 30% and 50% (such as 40%) by weight of a-dammar resin (soluble in alcohol); between 20% and 25% by weight (such as 22.5%) of b-dammar resin (insoluble in alcohol); between 20% and 30% by weight (such as 23.0%) of dammarol acid C₃₀H₅₀O₆ (a compound with one carboxyl and four hydroxyl groups); between 1% and 5% by weight (such as 2.5%) of water; between 2% and 10% by weight (such as 3.5%) of ash; between 5% and 15% by weight (such as 8.0%) of impurities; and between 0.1% and 1% by weight (such as 0.5%) of essential oils.

White dammars have a melting point m.p. of 80-90° C. (capillary), an acid number of 20-30 and a saponification number of 35-60; they are soluble in aromatic solvents, pentanol, pentyl acetate and carbon disulfide; sparingly soluble in other alcohols such as ethanol, ethyl acetate, ethers, ketones and aliphatic solvents. They are made compatible by heating with alkyd resins and oils.

According to a particular embodiment of the invention, the resin(s) are chosen from i) dammars.

### j) Elemis

“Elemis” is the generic term to define the group of recent natural resins derived from plants of the Burseraceae families (Canarium indicum). Each type is described according to its country of origin. According to a particular embodiment of the invention, the elemi resin used comes from the Philippines, notably Manila elemi. To extract it, the trees are wounded and a pathological resin flow appears, which solidifies with time. Elemis are yellowish to greenish, opaque, ointment-like, sticky, and solidify into brownish resins scattered with crystals.

Elemis are soluble in aromatic solvents, alcohols, esters and carbon disulfide; and less soluble in aliphatic solvents. Elemis have an acid number of 18 to 34, a saponification number of 25 to 60, and a softening point of about 80. The exuding balms of elemis contain up to 30% essential oils.

According to a preferred embodiment of the invention, the resin(s) of the invention are chosen from elemis, notably elemis from the Canarium luzonicum family in its pure form or in mixture with a latex, for example. Mention may be made of the elemi resin from Canarium luzonicum sold under the name RESINE ELEMI.

According to a particular embodiment of the invention, the resin(s) are chosen from j) elemis.

### k) Frankincenses (Olibanum)

Frankincenses are found in the United Arab Emirates, Oman, Somalia, Ethiopia and Eastern India. Frankincense resins are recent and are derived from the frankincense tree Boswellia carterii. Amazonian frankincense resins are also found. The bark is intentionally wounded to obtain a milky extract which is recovered after drying. Preferably the resin(s) of the invention are chosen from frankincenses, notably from Amazonia.

Frankincense resins are pale yellow, and form irregular rounded or globular beads. They generally contain 20% to 40% by weight (approx. 33%) of boswellic acid (C₃₂H₅₂O₄). Frankincenses have an acid number of between 30% and 50% (indirect) and are moderately soluble in ethanol in alkaline media.

According to a particular embodiment of the invention, the resin(s) of the invention are chosen from frankincense, notably Amazonian frankincense resins sold under the name Protium heptaphyllum resin, or PROTIUM RESIN, or WHITE BREU RESIN, and frankincense resins from the sal tree, Shorea robusta.

Advantageously, the resin(s) are mixed with one or more fatty substances as defined below according to the invention, preferably chosen from volatile or nonvolatile oils. Mention may be made, for example, of Shorea robusta resin with sunflower seed oil (SHOREA robusta RESIN, Helianthus annuus (SUNFLOWER) SEED OIL, TOCOPHEROL: 50-75% by weight Shorea robusta resin, 25-50% by weight sunflower seed oil) sold under the name KAHLRESIN 6720, and Shorea robusta resin with octyldodecanol (SHOREA ROBUSTA RESIN and OCTYLDODECANOL 50-70% by weight Shorea robusta resin, 30-50% by weight octyldodecanol) sold under the name KAHLRESIN 6720.

According to a particular embodiment of the invention, the resin(s) are chosen from k) frankincenses.

### l) Galbanums

Galbanum resins are recent resins obtained from various species of Iranian Ferula, for example Ferula galbaniflua.

Galbanums exude spontaneously from the bark like milk. They are collected as gum resins after drying. They are brownish to greenish-yellow, with regular bead or rounded tear shapes;

The older resins are darker.

Galbanums contain from 50% to 70% by weight (such as 60-65%) of resin, from 30% to 50% by weight (such as 35-40%) of plant gum, from 3% to 15% by weight (such as 6-10%) of essential oils; m.p.: 100° C.

According to a particular embodiment of the invention, the resin(s) are chosen from l) galbanums.

### m) Labdanums

Labdanum is a gum resin recently obtained from the leaves of various species of Cistacea (rock rose), a shrub of the Mediterranean region.

### n) Mastics

Mastic resins or mastics are recent resins harvested from the bark of Pistacia lentiscus. This shrub is found throughout the Mediterranean region. The resin is greenish yellow in colour and soluble in alcohols, aromatic solvents and terpenes. Its acid number is 50-70; saponification number 60-90; m.p. approx. 105° C. (capillary).

### o) Myrrh

Myrrh is a recent tree resin from various Commiphora species. The resin comes from Southern Arabia and Somalia and was known in biblical times. Myrrh is exuded from the plant in the form of milk and dries to form beads, which are then collected. Myrrh occurs as irregular, rounded beads or lumps of orange-brown colour with lighter areas and a bitter taste. A variety of myrrh oil can be found, and contains up to 10% (mixture of terpenes and sesquiterpenes).

### p) Sandarac

Sandarac (from ancient Greek σαvδαρ\acute{α}κη)/sandarákê, realgar) is a recent resin originating from Callitris quadrivalvis, a species of cypress found in Australia, North America and North Africa. It has a pleasant odour, is lemon-yellow in colour, is soluble in ethanol and in diethyl ether, and is insoluble in water. Sandarac has a m.p. 135° C. (capillary); an acid number of 115-150; and a saponification number of 145-155. The main ingredient of sandarac is sandaracopimaric acid (C₂₀H₃₀O₂).

### q) Shellacs

Shellacs are recent animal-based resins produced in India and Thailand. Shellac is produced by the female mealybug of the genus Kerria lacca, which lives in the forests of Southeast Asia (notably in the Assam region and Thailand). The insect settles on the trunks of trees (ficus and aleurites) and attaches itself by means of a resin that it secretes. They are collected from the empty shells of certain trees (Coccus laccaor, Laccifer lacca). The resins are then excreted from the empty shell juice. The word “laksham” means 100 000, as thousands of these shells cover the branches of trees (hence the term sticklac). Harvesting is done twice a year (April-May; November-December). Shellacs particularly contain between 50% and 90% by weight (preferably 60-80%) of pure shellac and from 1 to 10% by weight (preferably 4% to 6%) of shellac waxes. Purified resins are also known as pearl lacquers which comprise from 80% to 95% by weight (preferably from 85% to 90%) of pure shellac, and from 2% to 10% by weight (preferably from 5% to 8%) of shellac wax, and from 0.5% to 4% by weight (preferably from 2% to 3%) of impurities. They can be deparaffinned by dissolving them in aqueous sodium carbonate or by selective dissolution with certain solvents. Shellacs can be treated with alkali metal or alkaline-earth metal hypochlorite notably to obtain a light colour.

Shellacs are found in the form of bright orange or brown, shiny, transparent flakes. The different grades are Indian button (handmade) and shellac sheet; machine processed shellac sheet with or without wax; bleached shellac; and special shellac (water-soluble). Special shellacs have been pretreated with alkaline agents or amines, and can dissolve in water.

Shellacs have an m.p. of 65-77° C.; an acid number value of 67 to 90; and a saponification number between 190 and 260. They are soluble in ethanol, glycol ethers, acetic acid and alkaline water; and insoluble in aliphatics and aromatics. Shellacs attack iron in alcoholic solution. Some shellacs lose their solubility (over the years) due to polycondensation. The resins contain a hydroxycarboxylic acid bearing five hydroxyl groups, which leads to polycondensation.

r) Styrax (storax)

Styrax or “storax” is a recent resin isolated from the bark of Liquidambar orientalis (Hamamelidaceae family), produced in Asia Minor. Styrax is taken from the living tree once the tree has been wounded.

Styrax is often referred to as benzoic resin, it is aromatic, semi-solid and has a yellowish brown colour. It contains large amounts of cinnamic acid and derivatives thereof. Its acid number is about 105.

### s) Venice Turpentine (Larch, Turpentine Oil)

Recent Venice turpentine resin is extracted from the Larix europaea tree found in Europe.

### t) Colophony Resins

Preferably, the natural resin(s) are chosen from colophony resins (rosin). Colophony resins are recent resins, from renewable resources, and can be modified (for example esterified, hydrogenated or substituted).

Colophony gums are preferably purified, distilled from the balsam of various pine species (up to 80 different species).

Their composition is determined by climate, soil composition, and other botanical and meteorological factors. Examples that may be mentioned include colophony resins from Pinus austriaca (black pine) Austria, Central America, Pinus caribaea (slash pine), United States, Caribbean, Pinus densiflora Japan, Pinus elliottil United States, Pinus halepensis (Aleppo pine) Greece, Portugal, Spain, Pinus langifolia India, Pinus maritima (seashore pine) France, Spain, Portugal, Pinus massoniana (Chinese red pine) China, Pinus mercusii Indonesia, Burma, Philippines, Pinus nigra (black pine) Austria, Pinus oocarpa Central America, Honduras, Pinus palustris (swamp pine), United States, (longleaf pine), Pinus pseudostrobus Central America, Mexico, Pinus sylvestris (Scots pine) Germany, Poland, Pinus tonkinensis China, Pinus yunnanensis China.

The average composition is about 70% to 75% colophony resin and 20% to 25% turpentine.

### Wood Rosin [8050-09-7]

The rosin comes from stumps in the USA that have been left in the ground for at least 10 years so that the resin-rich heartwood is available.

The pine stumps contain between 10% and 30% by weight (approx. 19%) of rosin, between 1% and 10% by weight (preferably 4%) of turpentine oil, between 1% and 10% by weight (preferably 4%) of petroleum ether-insoluble resins, between 20% and 30% by weight (preferably 23%) of water and between 40% and 60% by weight (preferably 50%) of cellulose and lignin type.

According to a particular embodiment of the invention, the resin(s) are chosen from rosins.

### u) Rosin Tall Oils (Rosin and Rosinate) [8052-10-6]

Rosin tall oils often contain small amounts of higher fatty acids, particularly with a number of carbon atoms greater than or equal to 6 carbon atoms. According to one embodiment, rosin tall oils are free of oxocarboxylic acid. In particular, they are soluble in organic solvents.

The colophony resins of the invention in particular comprise rosin acids belonging to the terpenes. The numbering of the carbon atoms in the rosin acid molecules is shown using abietic acid as an example.

Rosin acids have the molecular chemical formula C₂₀H₃₀O₂ and thus belong to the diterpene family (four isoprene units). A large number of isomers of tricyclic rosin acids exist, which differ in the position of the two double bonds.

Advantageously, said resin according to the invention is chosen from: gum rosin obtained by incision on living trees, wood rosin which is extracted from the stumps or wood of pines, and tall oil (tall oil rosin) which is obtained from a by-product of paper production. Advantageously, said resin(s) include rosin acids; preferably mainly chosen from abietic-type and pimaric-type acids; and notably chosen from: levopimaric acid, neoabietic acid, abietic acid, dehydroabietic acid, tetrahydroabietic acid, dihydroabietic acid, dextropimaric acid, isodextropimaric acid; or palustric acid; and mixtures thereof.

The rosin derivatives may be derived in particular from the polymerization, hydrogenation and/or esterification (for example with polyhydric alcohols such as ethylene glycol, glycerol or pentaerythritol) of rosin acids. Mention may be made, for example, of the rosin esters sold under the reference Foral 85, Pentalyn H and Staybelite Ester 10 by the company Hercules; Sylvatac 95 and Zonester 85 by the company Arizona Chemical or Unirez 3013 by the company Union Camp.

According to one embodiment of the invention, the resin(s) are chosen from rosinates (salts of alkaline agents of rosin acids, notably salts of alkali metals such as sodium or potassium, alkaline-earth metals such as calcium, or metals such as zinc or magnesium).

According to another preferred embodiment of the invention, the resin(s) are chosen from rosin acid esters, notably esters of rosin acid as defined previously, and of (C₁-C₆)alkanol, polyhydroxy(C₁-C₆)alkane polyols such as glycerol, pentaerythritol, and mixtures thereof, more preferentially chosen from glyceryl rosinate sold under the name Resiester Gum A 35, glyceryl rosinate as a mixture with a hydrogenated plant oil and/or castor seed oil (Glyceryl rosinate, Ricinus communis seed oil, hydrogenated vegetable oil sold under the name EFP Biotek), pentaerythrityl rosinate sold under the name Resiester N 35 S and Resiester 80.

According to another embodiment of the invention, the resin(s) are chosen from adducts of poly(carboxy)(C₂-C₆)alkanes or poly(carboxy)(C₂-C₆)alkenes notably of maleic acids with rosin acids.

According to another embodiment of the invention, the resin(s) are chosen from rosins modified with phenols; in particular those modified with (C₁-C₄)alkylene phenols or diphenols, optionally substituted with one or more (C₁-C₄)alkyl groups such as methyl or t-butyl, more particularly rosins modified with 4-tert-butylphenol and 4,4′-isopropylidenediphenol (bisphenol A).

According to another embodiment of the invention, the resin(s) are chosen from dimerized rosins; notably those in which the abietic acid is polymerized. Preferably, the rosins contain more than 50% of dimer acids and are thus called dimerized rosins. According to one embodiment, the rosins are polymerized and contain from 30% to 90% by weight of dimer acid (notably at least 40%, 60% or 80% of dimer acids).

According to a preferred embodiment of the invention, the resin(s) are chosen from hydrogenated rosins. The double bonds, notably of acids such as abietic acid, are subject to oxidation, which can be eliminated by hydrogenation. It is understood that the resin(s) of the invention may be esterified, salified, adducted, modified with phenols, and/or dimerized and also hydrogenated.

According to a preferred embodiment, the resin comprises at least one rosin acid ester chosen from the group consisting of glyceryl rosinate, pentaerythrityl rosinate, silicone rosinate, diethylene glycol rosinate, dilinoleyl dimer hydrogenated rosinate, dipentaerythrityl hexahydroxystearate/hexastearate/hexarosinate, hydrogenated glyceryl dibehenate/rosinate, hydrogenated glyceryl diisostearate/rosinate, trihydrogenated glyceryl rosinate, glycol rosinate, hydrogenated methyl rosinate, methyl rosinate, hydrogenated pentaerythrityl rosinate, hydrogenated triethylene glycol rosinate and mixtures thereof.

According to a particular embodiment, the resin(s) of the invention are chosen from hydrogenated pentaerythrityl rosinate (pentaerythrityl hydrogenated rosinate), hydrogenated methyl rosinate (methyl hydrogenated rosinate) sold under the name Symrise Bio4326.

Furthermore, the resin(s) of the invention may be mixed with fatty substances c) as defined below, notably waxes or butters. Mention may be made of mixtures of glyceryl rosinate with one or more fatty substances c) notably chosen from waxes or butters such as the mixture with shea butter or olive oil such as (glyceryl rosinate, Ricinus communis seed oil, hydrogenated vegetable oil), Butyrospermum parkii (shea butter) glyceryl rosinate, Olea europaea (olive) oil unsaponifiables glyceryl rosinate, Olea europaea (olive) oil unsaponifiables sold by Shea Butter & Glyceryl Rosinate & Oils.

v) Resins Extracted from Plant Waxes

Natural plant waxes as such are not considered as resins. Although they are among the substances secreted/excreted by plants and naturally containing a very low resin content, they contain less than 30% by weight of terpenes relative to the total weight of wax. For example, carnauba wax is naturally secreted by the leaves of the Copernica cerifera palm to prevent the leaves from drying out. Candelilla wax is obtained from a shrub called Euphorbia antisyphilitica, which is native to Northern Mexico. The wax protects the plant from its environment and prevents excessive evaporation. For example, candelilla wax is mainly composed of hydrocarbons (about 50%, chains of 29 to 33 carbon atoms), higher molecular weight esters (20% to 29%), free acids (7% to 9%), and resins (12% to 14%, mainly triterpene esters).

However, for the purposes of the present invention, the definition of “natural resins” also includes resins derived from plant waxes, when they have been previously concentrated, isolated or extracted from these waxes, provided that the resinous or terpene ingredient under consideration contains the minimum terpene content (30% by weight relative to the total weight of the ingredient) required by the present invention. Mention may notably be made of candelilla resin (100% pure resin extracted from the corresponding wax), having the INCI name: Euphorbia cerifera (Candellila) Wax Extract, sold under the name Candelilla Resin E-1 by Japan Natural Products. WO 2013/147113 A1 also refers to carnauba resin, a terpene resin extracted from carnauba wax, and having physical properties similar to those of the natural resins classically described, such as a softening temperature rather than a melting point which differentiates the resin from the wax.

Resins have a softening point and a glass transition temperature, but no melting point.

The opposite is true for waxes, which have a melting point.

Preferably, the resin(s) are chosen from the resin(s) j), k), and t) as previously defined, and resins v) extracted from waxes, notably candelilla, Shorea robusta and beeswax (sold under the name Kahlwax 6721) or carnauba; and mixtures thereof, preferably resin extracted from candelilla wax.

### Preferred Resins According to the Invention:

Preferably, the natural resin(s) are chosen from j), k), t) and v).

According to a preferred embodiment of the invention, the resin(s) are chosen from the following references, indicated by their INCI name, used alone or as a mixture:
Euphorbia cerifera (candellila) wax extract, such as Candelilla Resin E-1 sold by Japan Natural Products, Botanical Resin sold by Cera Rica Noda, Towax-1F12 sold by Toa Kasei (type v resin);
Protium heptaphylum resin, or protium resin, or white breu resin, sold for example by Citroleo or Ephyla (type k resin)
Frankincense resins from the sal tree, Shorea robusta resin. The resin(s) may be mixed with one or more fatty substances c) as defined below, preferably chosen from volatile oils or nonvolatile oils. An example is Shorea robusta resin with sunflower seed oil (Shorea robusta resin, Helianthus annuus (sunflower) seed oil, tocopherol: 50-75% by weight Shorea robusta resin, 25-50% by weight sunflower seed oil) sold under the name Kahlresin 6720, and Shorea robusta resin with octyldodecanol (Shorea robusta resin and octyldodecanol 50-70% by weight Shorea robusta resin, 30-50% by weight octyldodecanol) sold by Kahlresin 6720 (type k resin)
rosin acid esters such as glyceryl rosinate sold under the name Resiester Gum A 35, glyceryl rosinate as a mixture with hydrogenated plant oil and/or castor seed oil (glyceryl rosinate, Ricinus communis seed oil, hydrogenated vegetable oil sold under the name EFP Biotek) pentaerythrityl rosinate sold under the name Resiester N 35 S and Resiester 80 or hydrogenated rosinates such as hydrogenated pentaerythrityl rosinate (pentaerythrityl hydrogenated rosinate), hydrogenated methyl rosinate (methyl hydrogenated rosinate) sold under the name Symrise B104326 (t type resin).

According to a preferred embodiment of the invention, the resin(s) are chosen from Euphorbia cerifera (candelilla) wax extract.

Advantageously, the resin(s) are present in the composition of the invention in a content in the range from 0.01% to 40%, preferably from 0.15% to 35%, preferably from 0.51% to 30%, preferably from 12% to 25%, preferably from 3% to 22% and advantageously from 5% to 20%, by weight relative to the total weight of the composition representing 100%.

Advantageously, the composition of the present invention comprises less than 10%, preferably less than 5%, preferably less than 1%, preferably less than 0.5%, preferably less than 0.1%, and is preferably free, of synthetic resin.

Advantageously, the composition of the present invention comprises less than 10%, preferably less than 5%, preferably less than 1%, preferably less than 0.5%, preferably less than 0.1%, and is preferably free, of silicone resin, i.e. synthetic resin in which the basic structure is a chain including siloxane groups (silicon-oxygen-silicon bonds). Preferably, the resin(s) are chosen from the resin(s) j), k) and t) as defined previously.

According to another preferred embodiment, the resin(s) of the invention are present in the composition in an amount of between 0.01% and 30% by weight relative to the total weight of the composition, more particularly between 0.1% and 20% by weight, more particularly between 0.5% and 12.5% by weight relative to the total weight of the composition.

### c) The Fatty Substances

According to a particular embodiment of the invention, the composition also comprises one or more fatty substances.

The term “fatty substance” means an organic compound that is insoluble in water at ordinary room temperature (25° C.) and at atmospheric pressure (760 mmHg) (solubility of less than 5%, preferably 1% and even more preferentially 0.1%). They bear in their structure at least one hydrocarbon-based chain including at least 6 carbon atoms or a sequence of at least two siloxane groups. In addition, the fatty substances are generally soluble in organic solvents under the same temperature and pressure conditions, for instance chloroform, ethanol, benzene, liquid petroleum jelly or decamethylcyclopentasiloxane.

The fatty substance(s) of the invention are of natural or synthetic origin, preferably natural, more preferentially of plant origin. They are different from fatty acids since salified fatty acids constitute soaps which are generally soluble in aqueous media.

According to a particular embodiment of the invention, the composition comprises one or more fatty substances that are not liquid at 25° C. and at atmospheric pressure.

### The Wax(es)

According to a particular embodiment, the composition of the invention comprises one or more waxes.

The term “wax” means a lipophilic compound that is solid at room temperature (25° C.), with a reversible solid/liquid change of state, having a melting point of greater than or equal to 30° C., which may be up to 200° C. and notably up to 120° C.

In particular, the wax(es) that are suitable for use in the invention may have a melting point of greater than or equal to 45° C. and in particular of greater than or equal to 55° C.

The composition according to the invention preferably comprises a content of wax(es) ranging from 0.5% to 30% by weight relative to the total weight of the composition, in particular from 1% to 20% and more particularly from 2% to 15%.

According to a particular form of the invention, the composition of the invention is solid, in particular anhydrous. It may then be in stick form; use will be made of polyethylene microwaxes in the form of crystallites with an aspect ratio at least equal to 2, and with a melting point ranging from 70 to 110° C. and preferably from 70 to 100° C., so as to reduce or even eliminate the presence of strata in the solid composition. These crystallites in needle form and notably the dimensions thereof may be characterized visually according to the following method.

### The Pasty Compound(s)

According to a particular embodiment, the composition of the invention comprises one or more pasty compounds.

For the purposes of the present invention, the term “pasty compound” means a lipophilic fatty compound that undergoes a reversible solid/liquid change of state, having anisotropic crystal organization in the solid state, and including, at a temperature of 23° C., a liquid fraction and a solid fraction.

Preferably, the composition contains one or more fatty substances c) which are hydrocarbon-based fatty substances that are liquid at 25° C. and atmospheric pressure.

The hydrocarbon-based liquid fatty substance(s) are notably chosen from C₆-C₁₆ hydrocarbons or hydrocarbons comprising more than 16 carbon atoms and up to 60 carbon atoms, preferably between C₆ and C₁₆, and in particular alkanes, oils of animal origin, oils of plant origin, glycerides or fluoro oils of synthetic origin, fatty alcohols, fatty acid and/or fatty alcohol esters, and silicones. In particular, the liquid fatty substance(s) are chosen from non-silicone oils.

It is recalled that, for the purposes of the invention, the fatty alcohols, fatty esters and fatty acids more particularly contain one or more linear or branched, saturated or unsaturated hydrocarbon-based groups comprising 6 to 60 carbon atoms, which are optionally substituted, in particular with one or more hydroxyl groups OH (in particular from 1 to 4 hydroxyl groups). If they are unsaturated, these compounds may comprise one to three unsaturations, preferably from one to three conjugated or unconjugated carbon-carbon double bonds.

As regards the C₆-C₁₆ alkanes, these compounds are linear or branched, and optionally cyclic; preferably, the fatty substance(s) c) of the invention are chosen from linear or branched C₈-C₁₄, more preferentially C₉-C₁₃ and even more preferentially C₉-C₁₂ alkanes. Examples that may be mentioned include hexane, decane, undecane, dodecane, tridecane, and isoparaffins, for instance isohexadecane, isodecane or isododecane. The linear or branched hydrocarbons containing more than 16 carbon atoms may be chosen from liquid paraffins, liquid petroleum jelly, polydecenes, and hydrogenated polyisobutene such as Parleam®.

Among the hydrocarbon-based liquid fatty substances c) having an overall solubility parameter according to the Hansen solubility space of less than or equal to 20 (MPa)^{1/2}, mention may be made of oils, which may be chosen from natural or synthetic, hydrocarbon-based oils, which are optionally fluorinated and optionally branched, alone or as a mixture.

According to a very advantageous embodiment, the composition of the invention comprises one or more fatty substances which are one or more hydrocarbon-based oils. The hydrocarbon-based oil(s) may be volatile or non-volatile.

According to a preferred embodiment of the invention, the fatty substance(s) c) are linear or branched hydrocarbon-based oils, which are volatile, notably chosen from undecane, decane, dodecane, isododecane, tridecane, and a mixture of various volatile oils thereof preferably comprising isododecane in the mixture, or a mixture of undecane and tridecane.

According to another particular embodiment, the liquid fatty substance(s) c) are a mixture of a volatile hydrocarbon-based oil and a non-volatile hydrocarbon-based oil, the mixture of which preferentially comprises dodecane or isododecane as volatile oil.

In particular, the fatty substance(s) c) of the invention are a mixture of C₉-C₁₂ alkanes, preferably of natural origin, the chains of which comprise from 9 to 12 carbon atoms, preferably linear or branched C₉-C₁₂ alkanes. This mixture is notably known under the INCI name C₉-C₁₂ Alkane, CAS 68608-12-8, Vegelight Silk® sold by BioSynthls. This volatile biodegradable mixture of volatile oils is obtained from coconut oil (the viscosity is 0.9-1.1 cSt (40° C.) and it has a flash point of 65° C.).

According to one embodiment, the composition contains only oils that are liquid at 25° C. and atmospheric pressure. According to another embodiment, the composition contains at least 80% of hydrocarbon-based oils that are liquid at 25° C. and atmospheric pressure, which are preferably volatile, more preferentially chosen from isodecane, decane, Cetiol UT® and Vegelight Silk®.

According to another embodiment, the composition may comprise volatile and non-volatile oils, notably in a volatile oil/non-volatile oil ratio of greater than or equal to 4.

According to another embodiment, the composition contains from 0 to 10% of silicone oils, preferably from 0 to 5% of silicone oils.

Volatile silicone oils that may be mentioned include volatile linear or cyclic silicone oils, notably those with a viscosity of less than or equal to 8 centistokes (cSt) (8×10⁻⁶ m²/s), and notably containing from 2 to 10 silicon atoms and in particular from 2 to 7 silicon atoms, these silicones optionally including alkyl or alkoxy groups containing from 1 to 10 carbon atoms. As volatile silicone oils that may be used in the invention, mention may notably be made of dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

As nonvolatile silicone oils, mention may be made of linear or cyclic nonvolatile polydimethylsiloxanes (PDMSs); polydimethylsiloxanes including alkyl, alkoxy and/or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes, 2-phenylethyl trimethylsiloxysilicates and pentaphenyl silicone oils.

The hydrocarbon-based oil may be chosen from:
hydrocarbon-based oils containing from 8 to 14 carbon atoms, and notably:
branched C₈-C₁₄ alkanes, for instance C₈-C₁₄ isoalkanes of petroleum origin (also known as isoparaffins), for instance isododecane (also known as 2,2,4,4,6-pentamethylheptane), isodecane and, for example, the oils sold under the trade names Isopar or Permethyl,
linear alkanes, for instance n-dodecane (C12) and n-tetradecane (C14) sold by Sasol under the respective references Parafol 12-97 and Parafol 14-97, and also mixtures thereof, the undecane-tridecane mixture, the mixtures of n-undecane (C11) and of n-tridecane (C13) obtained in examples 1 and 2 of patent application WO 2008/155059 from the company Cognis, and mixtures thereof, and also mixtures of n-undecane (C11) and of n-tridecane (C13) Cetiol Ultimate® from the company BASF,
short-chain esters (containing from 3 to 8 carbon atoms in total) such as ethyl acetate, methyl acetate, propyl acetate or n-butyl acetate,
hydrocarbon-based oils of plant origin such as triglycerides consisting of fatty acid esters of glycerol, the fatty acids of which may have various chain lengths ranging from C₄ to C₂₄, these chains possibly being linear or branched, and saturated or unsaturated; these oils are notably heptanoic acid or octanoic acid triglycerides, or alternatively wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cotton oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passion flower oil, musk rose oil or coconut oil; shea butter; or else caprylic/capric acid triglycerides, for instance those sold by the company Stéarinerie Dubois or those sold under the names Miglyol 810®, 812® and 818® by the company Dynamit Nobel,
synthetic ethers containing from 10 to 40 carbon atoms,
linear or branched hydrocarbons of mineral or synthetic origin, such as petroleum jelly, polydecenes, hydrogenated polyisobutene such as Parleam®, squalane and liquid paraffins, and mixtures thereof,
esters such as the oils of formula R¹C(O)—O—R² in which R¹ represents a linear or branched fatty acid residue including from 1 to 40 carbon atoms and R² represents a, notably branched, hydrocarbon-based chain containing from 1 to 40 carbon atoms, on condition that R¹+R² is greater than or equal to 10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, 2-hexyldecyl laurate, 2-octyldecyl palmitate, 2-octyldodecyl myristate, alcohol or polyalcohol heptanoates, octanoates, decanoates or ricinoleates such as propylene glycol dioctanoate; hydroxylated esters such as isostearyl lactate, diisostearyl malate and 2-octyldodecyl lactate; polyol esters and pentaerythritol esters, more preferentially esters of a linear or branched C₈-C₁₀ fatty acid and of a linear or branched C₁₂-C₁₈ fatty alcohol alone or as a mixture with alkanes derived from the complete hydrogenation/reduction of fatty acids obtained from Cocos nucifera (coconut) oil, particularly dodecane or mixtures of cocoyl caprylate/caprate with dodecane; mention may be made of those having the INCI name Coconut alkanes (and) cocoyl caprylate/caprate sold under the name Vegelight 1212LC® by Grant Industries,
fatty alcohols that are liquid at room temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol and 2-undecylpentadecanol.

In addition to the hydrocarbon-based liquid fatty substance, the composition of the invention may comprise a silicone oil. If silicone oil is in the composition of the invention, it is preferably in an amount which does not exceed 10% by weight relative to the weight of the composition, more particularly in an amount of less than 5% and more preferentially less than 2% by weight relative to the total weight of the composition.

In particular, the composition comprises at least one hydrocarbon-based liquid fatty substance c) chosen from:
plant oils formed by fatty acid esters of polyols, in particular triglycerides, such as sunflower oil, sesame oil, rapeseed oil, macadamia oil, soybean oil, sweet almond oil, beauty-leaf oil, palm oil, grapeseed oil, corn oil, arara oil, cottonseed oil, apricot oil, avocado oil, jojoba oil, olive oil, coconut oil or cereal germ oil;
linear, branched or cyclic esters containing more than 6 carbon atoms, notably 6 to 30 carbon atoms; and notably isononyl isononanoate;
and more particularly esters of formula R^{d}—C(O)—O—R^{e} in which R^{d} represents a higher fatty acid residue including from 7 to 19 carbon atoms and R^{e} represents a hydrocarbon-based chain including from 3 to 20 carbon atoms, such as palmitates, adipates, myristates and benzoates, notably diisopropyl adipate and isopropyl myristate; more preferentially esters of formula R^{d}—C(O)—O—R^{e} in which R^{d} represents a higher fatty acid residue including from 8 to 10 carbon atoms and R^{e} represents a hydrocarbon-based chain including from 12 to 18 carbon atoms;
hydrocarbons and notably volatile or non-volatile linear, branched and/or cyclic alkanes, such as optionally volatile C₅-C₆₀ isoparaffins, such as undecane, dodecane, isododecane, tridecane, Parleam (hydrogenated polyisobutene), isohexadecane, cyclohexane, or Isopars, and mixtures thereof; or alkanes resulting from the complete hydrogenation/reduction of mixtures of fatty acids derived from Cocos nucifera (coconut) oil, such as dodecane, the mixture of C₉-C₁₂ alkanes, the chains of which comprise from 9 to 12 carbon atoms, preferably linear or branched C₉-C₁₂ alkanes, in particular comprising dodecane, or else liquid paraffin, liquid petroleum jelly, or hydrogenated polyisobutylene;
ethers containing 6 to 30 carbon atoms;
ketones containing 6 to 30 carbon atoms;
aliphatic fatty monoalcohols containing 6 to 30 carbon atoms, the hydrocarbon-based chain not including any substitution groups, such as oleyl alcohol, decanol, dodecanol, octadecanol, octyldodecanol and linoleyl alcohol;
polyols containing 6 to 30 carbon atoms, such as hexylene glycol; and
mixtures thereof, such as mixtures of esters of linear or branched C₈-C₁₀ fatty acid and C₁₂-C₁₈ fatty alcohol and alkanes resulting from the complete hydrogenation/reduction of mixtures of fatty acids from Cocos nucifera (coconut) oil, in particular dodecane, such as mixtures of cococaprylate/caprate and dodecane; mention may be made of those having the INCI name Coconut alkanes (and) coco-caprylate/caprate sold under the name Vegelight 1212LC® by Grant Industries; or mixtures of C₉-C₁₂ alkanes, the chains of which comprise from 9 to 12 carbon atoms, preferably linear or branched C₉-C₁₂ alkanes, in particular comprising dodecane; mention may be made of the oil mixture having the INCI name C9-12 Alkane, Vegelight Silk® sold by BioSynthIs.

Preferably, the composition of the invention comprises at least one hydrocarbon-based liquid fatty substance c) chosen from:
plant oils formed by fatty acid esters of polyols, in particular triglycerides,
esters of formula R^{d}—C(O)—O—R^{e} in which R^{d} represents a higher fatty acid residue including from 7 to 19 carbon atoms and R^{e} represents a hydrocarbon-based chain including from 3 to 20 carbon atoms, more preferentially esters of formula R^{d}—C(O)—O—R^{e} in which R^{d} represents a higher fatty acid residue including from 8 to 10 carbon atoms and R^{e} represents a hydrocarbon-based chain including from 12 to 18 carbon atoms;
volatile or non-volatile, linear or branched C₈-C₆₀ alkanes, such as isododecane and alkanes resulting from the complete hydrogenation/reduction of mixtures of fatty acids obtained from Cocos nucifera (coconut) oil, in particular dodecane;
volatile or non-volatile, non-aromatic cyclic C₅-C₁₂ alkanes;
ethers containing 7 to 30 carbon atoms;
ketones containing 8 to 30 carbon atoms;
aliphatic fatty monoalcohols containing 12 to 30 carbon atoms, the hydrocarbon-based chain not including any substitution groups; and
mixtures thereof.

Advantageously, the fatty substance(s) c) of the invention, which are notably liquid, are apolar, i.e. formed solely of carbon and hydrogen atoms.

The hydrocarbon-based liquid fatty substance(s) are preferably chosen from hydrocarbon-based oils containing from 8 to 14 carbon atoms, which are in particular volatile, more particularly the apolar oils described previously.

Preferentially, the fatty substance(s) c) of the invention, which are notably liquid, are chosen from alkanes, such as C₁₅-C₁₉ alkanes, dodecane, decane, isododecane, hydrogenated polyisobutene, fatty alcohols such as octyldodecanol, esters such as isononyl isononanoate, cocoyl caprylate/caprate and mixtures thereof, more preferentially alkanes.

More particularly, the fatty substance(s) c) of the invention, which are notably liquid, are chosen from linear or branched C₆-C₁₉ alkanes, such as C₁₅-C₁₉ alkanes, preferably from linear or branched C₆-C₁₆, preferably C₈-C₁₄, more preferentially C₉-C₁₃ and even more preferentially C₉-C₁₂ alkanes, and even more preferentially the alkanes are volatile. More particularly, the liquid fatty substance(s) iii) of the invention are volatile and are chosen from undecane, decane, dodecane, isododecane, tridecane, and a mixture thereof notably comprising dodecane, isododecane or a mixture of undecane and tridecane.

Preferentially, the liquid fatty substance(s) c) of the invention, which are notably liquid, are isododecane.

According to another advantageous embodiment of the invention, the fatty substance(s) c) of the invention, which are notably liquid, are a mixture of non-volatile oil(s) and volatile oil(s); preferably, the mixture comprises, as volatile oil, undecane, dodecane, isododecane or tridecane, more preferentially isododecane. A mixture of volatile oil and non-volatile oil that may be mentioned is the mixture of isododecane and of isononyl isononanoate or the mixture of isododecane with isononyl isononanoate.

Preferably, the composition comprises one or more fatty substances chosen from the waxes and butters as defined previously. More particularly, the composition comprises a mixture of oil(s)+butter(s), oil(s)+wax(es), or oil(s)+butter(s)+wax(es) fatty substances.

More preferentially, when the fatty substance(s) are a mixture of volatile oil and of non-volatile oil, the amount of volatile oil is greater than the amount of non-volatile oil.

In particular, in the mixture, the non-volatile oil is a phenyl silicone oil, preferably chosen from pentaphenyl silicone oils.

Advantageously, the composition comprises one or more fatty substances, which are notably liquid at 25° C. and at atmospheric pressure, preferably one or more oils, in a content ranging from 2% to 99.9% by weight, relative to the total weight of the composition, preferably ranging from 5% to 90% by weight, preferably ranging from 10% to 80% by weight, preferably ranging from 20% to 80% by weight.

According to a preferred embodiment of the invention, the composition according to the invention comprises c) one or more fatty substances that are notably liquid at 25° C. and at atmospheric pressure, e) water and f) one or more water-miscible solvents.

d) Organic Solvent(s) Other than c)

According to a particular embodiment of the invention, the composition also comprises one or more organic solvents other than c), which are apolar or polar, preferably polar, and which are protic or aprotic, more particularly protic and/or polar, preferably protic and polar.

Preferably, the organic solvent(s) are water-miscible.

According to the invention, the term “water-miscible solvent” is intended to denote a compound that is liquid at room temperature and water-miscible (water miscible the of greater than 50% by weight at 25° C. and atmospheric pressure).

The organic solvent(s) may be used in the composition of the invention may also be volatile.

Among the organic solvents that may be used in the composition according to the invention, mention may notably be made of polar protic or polar aprotic organic solvents, preferably polar protic organic solvents, particularly lower monoalcohols containing from 2 to 10 carbon atoms, such as ethanol and isopropanol, preferably ethanol.

According to one embodiment, the composition of the invention comprises one or more organic solvents, preferably chosen from monoalcohols containing from 2 to 6 carbon atoms such as ethanol and isopropanol.

Preferably, the amount of organic solvent(s) is less than 70% by weight, more preferentially less than 50% by weight, relative to the total weight of the composition. According to one embodiment of the invention, the composition comprises an amount of organic solvent(s) of greater than 0.5%, more particularly greater than or equal to 1% by weight relative to the total weight of the composition. In particular, the composition comprises between 2% and 50% by weight of organic solvent(s).

### e) Water

According to a particular embodiment of the invention, the composition also comprises water.

The water that is suitable for use in the invention may be tap water, distilled water, spring water, a floral water such as cornflower water and/or a mineral water such as Vittel water, Lucas water or La Roche Posay water and/or a thermal water.

According to one embodiment, the composition of the invention comprises e) water and at least one fatty substance c) in a ratio between the mass of water and the mass of fatty substance c) of less than 1, preferably less than 0.9, more preferentially less than 0.9, such as between 0.5 and 0.8.

According to a particular embodiment of the invention, the composition comprises an amount of water of less than or equal to 5% by weight relative to the total weight of the composition, particularly less than or equal to 2% by weight, preferably less than 1% by weight, more preferentially less than 0.5% by weight relative to the total weight of the composition. More particularly, the composition of the invention is anhydrous, i.e. it is free of water.

### f) Surfactants

According to a particular embodiment of the invention, the composition also comprises f) one or more surfactants, preferably nonionic or ionic surfactants, or mixtures thereof.

According to another particular embodiment of the invention, the composition does not comprise any surfactant.

The term “surfactant” means a compound which modifies the surface tension between two surfaces. The surfactant(s) d) are amphiphilic molecules, which have two parts of different polarity, one part being lipophilic (which retains fatty substances) which is apolar, the other hydrophilic part (miscible or soluble in water) being polar. The lipophilic part is generally a fatty chain, and the other water-miscible part is polar, and/or protic.

The term “ionic” means anionic, cationic, amphoteric or zwitterionic.

The term “fatty chain” means a linear or branched, saturated or unsaturated hydrocarbon-based chain comprising more than 6 atoms, preferably between 6 and 30 carbon atoms and preferably from 8 to 24 carbon atoms.

According to a first particular embodiment, the composition of the invention contains d) at least one silicone or non-silicone nonionic surfactant.

Among the nonionic surfactants according to the invention, mention may be made, alone or as mixtures, of fatty alcohols, α-diols and alkylphenols, these three types of compound being polyethoxylated, polypropoxylated and/or polyglycerolated and containing a fatty chain comprising, for example, 8 to 22 carbon atoms, the number of ethylene oxide or propylene oxide groups possibly ranging in particular from 2 to 50 and the number of glycerol groups possibly ranging in particular from 2 to 30. Mention may also be made of ethylene oxide and propylene oxide copolymers, condensates of ethylene oxide and of propylene oxide with fatty alcohols; polyethoxylated fatty amides preferably having from 2 to 30 ethylene oxide units, polyglycerolated fatty amides containing on average 1 to 5, and in particular 1.5 to 4, glycerol groups, ethoxylated fatty acid esters of sorbitan containing from 2 to 30 ethylene oxide units, fatty acid esters of sucrose, fatty acid esters of polyethylene glycol, alkylpolyglycosides, N-alkylglucamine derivatives, amine oxides such as (C₁₀-C₁₄)alkylamine oxides or N-acylaminopropylmorpholine oxides.

The surfactant(s) represent in total particularly from 0.01% to 30% by weight, preferably from 0.5% to 15% by weight, even more preferentially from 1% to 10% by weight and better still between 1% and 5% by weight of the composition, relative to the total weight of the composition.

### Form of the Composition:

According to one embodiment of the invention, the composition comprises an aqueous phase. The composition is notably formulated as aqueous lotions or as water-in-oil or oil-in-water emulsions or as multiple emulsions (oil-in-water-in-oil or water-in-oil-in-water triple emulsions (such emulsions are known and described, for example, by C. Fox in “Cosmetics and Toiletries”—November 1986—Vol. 101—pages 101-112)).

According to a particular embodiment of the invention, the composition is a direct emulsion, i.e. an emulsion of oil-in-water or O/W type. The weight amount of oil is preferably less than 70% in the inverse emulsion, preferably less than or equal to 40%, more particularly less than or equal to 35% by weight relative to the total weight of the composition.

More particularly, in the direct emulsion, the amount of water is greater than or equal to 30% by weight relative to the total weight of the composition, more particularly greater than or equal to 40%, preferentially greater than or equal to 35%.

According to another particular embodiment of the invention, the composition of the invention is an inverse emulsion, i.e. of water-in-oil or W/O type. The weight amount of oil is preferably greater than 30% in the inverse emulsion, preferably greater than 40%, more preferentially greater than or equal to 45% by weight relative to the total weight of the composition. More particularly, in the inverse emulsion, the amount of water is less than 40% by weight relative to the total weight of the composition, preferably less than or equal to 35% by weight.

The composition according to the invention preferably has a pH ranging from 3 to 9, depending on the support chosen.

According to a particular embodiment of the invention, the pH of the composition(s) is neutral or even slightly acidic. Preferably, the pH of the composition is between 6 and 7. The pH of these compositions may be adjusted to the desired value by means of acidifying or basifying agents usually used in cosmetics, or alternatively using standard buffer systems.

The term “basifying agent” or “base” means any agent for increasing the pH of the composition in which it is present. The basifying agent is a Brønsted, Lowry or Lewis base. It may be mineral or organic. Particularly, said agent is chosen from a) aqueous ammonia, b) (bi)carbonate, c) alkanolamines such as monoethanolamine, diethanolamine, triethanolamine and derivatives thereof, d) oxyethylenated and/or oxypropylenated ethylenediamines, e) organic amines, f) mineral or organic hydroxides, g) alkali metal silicates such as sodium metasilicates, h) amino acids, preferably basic amino acids such as arginine, lysine, ornithine, citrulline and histidine, and i) the compounds of formula (F) below:

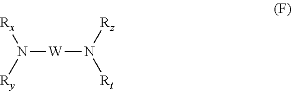

in which formula (F):
   W is a divalent C₁-C₆ alkylene radical optionally substituted with one or more hydroxyl groups or a C₁-C₆ alkyl radical, and/or optionally interrupted with one or more heteroatoms such as O or NRᵤ;
   Rₓ, R_{y}, R_{z}, Rₜ and Rᵤ, which may be identical or different, represent a hydrogen atom or a C₁-C₆ alkyl, C₁-C₆ hydroxyalkyl or C₁-C₆ aminoalkyl radical.

Examples of amines of formula (F) that may be mentioned include 1,3-diaminopropane, 1,3-diamino-2-propanol, spermine and spermidine.

The term “alkanolamine” means an organic amine comprising a primary, secondary or tertiary amine function, and one or more linear or branched C₁—C alkyl groups bearing one or more hydroxyl radicals.

Among the mineral or organic hydroxides, mention may be made of those chosen from a) hydroxides of an alkali metal, b) hydroxides of an alkaline-earth metal, for instance sodium hydroxide or potassium hydroxide, c) hydroxides of a transition metal, d) hydroxides of lanthanides or actinides, quaternary ammonium hydroxides and guanidinium hydroxide. The mineral or organic hydroxides a) and b) are preferred.

Mention may be made, among the acidifying agents for the compositions used in the invention, by way of example, of mineral or organic acids, such as hydrochloric acid, orthophosphoric acid, sulfuric acid, carboxylic acids, such as acetic acid, tartaric acid, citric acid or lactic acid, or sulfonic acids.

The basifying agents and the acidifying agents as defined previously preferably represent from 0.001% to 20% by weight relative to the weight of the composition. and more particularly from 0.005% to 8% by weight of the composition.

According to a particular embodiment of the invention, composition comprises an amount of water of less than or equal to 10% by weight relative to the total weight of the composition. Even more preferentially, composition comprises an amount of water of less than or equal to 5%, better still less than 2%, even better still less than 0.5%, and is notably free of water. Where appropriate, such small amounts of water may notably be introduced by ingredients of the composition that may contain residual amounts thereof.

According to a particular embodiment of the invention, the composition does not comprise any water.

Advantageously, composition C1 according to the invention comprises a physiologically acceptable medium. In particular, the composition is a cosmetic composition.

The term “physiologically acceptable medium” means a medium that is compatible with human keratin materials, for instance the skin, the lips, the nails, the eyelashes, the eyebrows or the hair.

The term “cosmetic composition” means a composition that is compatible with keratin materials, which has a pleasant colour, odour and feel and which does not cause any unacceptable discomfort (stinging or tautness) liable to discourage the consumer from using it.

The term “keratin materials” means the skin (body, face, contour of the eyes, scalp), head hair, the eyelashes, the eyebrows, bodily hair, the nails or the lips.

The composition according to the invention may comprise one or more cosmetic additives chosen from fragrances, preserving agents, fillers, colouring agents, UV-screening agents, oils other than the fatty substances c), moisturizers, vitamins, ceramides, antioxidants, free-radical scavengers, polymers other than a), thickeners or film-forming agents other than b), trace elements, softeners, sequestrants, agents for combating hair loss, anti-dandruff agents, propellants. In particular, the composition according to the invention also comprises one or more colouring agents chosen from pigments, direct dyes and mixtures thereof, preferably pigments; more preferentially, the pigment(s) of the invention are chosen from carbon black, iron oxides, notably black iron oxides, and micas coated with iron oxide, triarylmethane pigments, notably blue and violet triarylmethane pigments, such as Blue 1 Lake, azo pigments, notably red azo pigments, such as D&C Red 7, an alkali metal salt of lithol red, such as the calcium salt of lithol red B, even more preferentially red iron oxides.

Advantageously, the composition according to the invention is a makeup composition, in particular a lip makeup composition, a mascara, an eyeliner, an eye shadow or a foundation.

### Additional Solvents

According to a particular embodiment of the invention, the composition comprises one or more solvents, preferably polar and/or protic solvents, other than water in the predominantly fatty medium.

### The Adjuvants

The composition according to the invention may also comprise one or more fillers, notably in a content ranging from 0.01% to 30% by weight and preferably ranging from 0.01% to 20% by weight relative to the total weight of the composition. The term “fillers” should be understood as meaning colourless or white, mineral or synthetic particles of any shape, which are insoluble in the medium of the composition, irrespective of the temperature at which the composition is manufactured. These fillers notably serve to modify the rheology or texture of the composition.

The composition according to the invention may be in the form of an anhydrous composition, a water-in-oil emulsion or an oil-in-water emulsion.

The invention is illustrated in greater detail in the examples that follow. The amounts are indicated as weight percentages.

### EXAMPLES

The PHAs illustrated in the various examples were prepared in 3-litre chemostats and/or 5-litre Fernbach flasks depending on whether or not a 3-oxidation pathway inhibitor was used. The isolation of the PHAs is similar for all the examples obtained.

In a first step, the microorganism generates the PHAs which are stored in intracellular granules, the proportion of which varies as a function of the applied conditions such as the temperature or the nature of the culture medium. The generation of PHA granules may or may not be associated with the growth of the microorganism as a function of the nature of the microorganisms. During the second step, the biomass containing the PHAs is isolated, i.e. separated from the fermentation medium, and then dried. The PHAs are extracted from the biomass before being purified, if necessary.

A mixture of saturated and unsaturated carbon sources is, for certain examples, necessary for the stability of the PHA obtained.

TABLE 2

| Carbon source | CAS |
|---|---|
| Caprylic acid (RADIACID 608) | 124-07-2 |
| Nonanoic acid | 112-05-0 |
| Undecylenic acid (10-Undecenoic acid) | 112-38-9 |

TABLE 3

| Carbon source | Genus and species | Source |
|---|---|---|
| Mixture of caprylic acid | Pseudomonas | ATCC ® 47054 ™ |
| and undecylenic acid | putida | |
| Mixture of nonanoic acid | Pseudomonas | ATCC ® 47054 ™ |
| and undecylenic acid | putida | |

### Example 1: PHA Bearing a Side Chain R¹ Representing a Linear 10% Unsaturated n-octenyl Group and R² Representing an n-pentyl Group

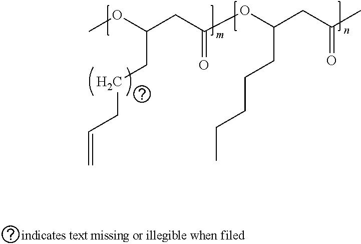

The process for synthesizing the compound of Example 1 is adapted from the article: Fed-batch production of unsaturated medium-chain-length polyhydroxyalkanoates with controlled composition by Pseudomonas putida KT2440, Z. Sun, J. A. Ramsay, M. Guay, B. A. Ramsay, Applied Microbiology Biotechnology, 82. 657-662, 2009.

The microorganism used is Pseudomonas putida KT2440 ATCC®47054™. The culture method is performed under fed-batch growth axenic conditions with a maintenance solution containing a mixture of carbon source at a rate μ=0.15 h⁻¹ in a 3 L chemostat containing 2.5 L of culture medium.

The system is aerated with a flow of 0.5 vvm of air for a nominal dissolved oxygen (O_{D}) value at 30% of saturation. The pH is regulated with 15% aqueous ammonia solution. The temperature of the fermentation medium is regulated at 30° C.

### Equipment for the Fed-Batch Growth Fermentation Mode:

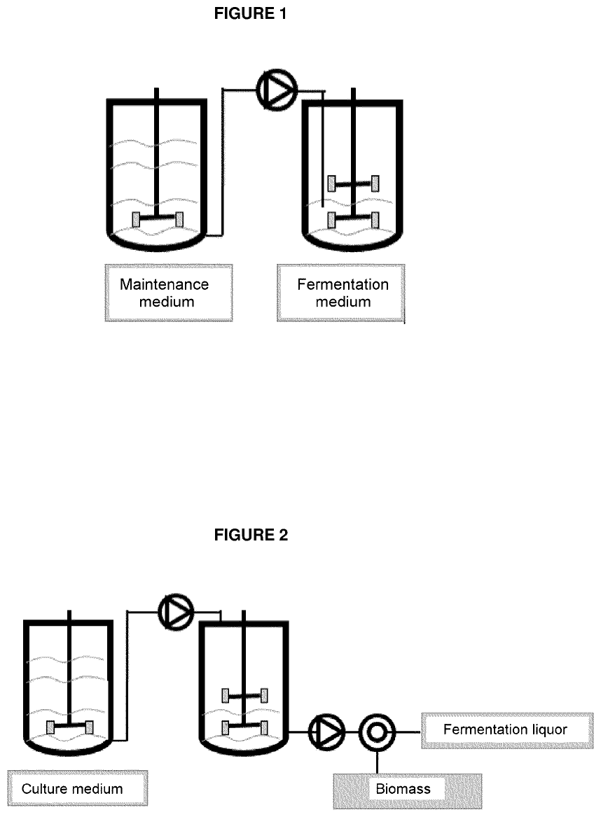

The fermentation medium is regulated in terms of temperature-pressure of dissolved oxygen and pH (not shown): see the attached FIG. 1.

The production process is performed using three different culture media. The first culture medium, defined CM1 “inoculum”, is used for the preparation of the preculture. The second culture medium, defined CM2 “batch”, is used for unfed batch growth of the microorganism with the primary carbon sources in the Fernbach flasks. The third culture medium, defined CM3 “maintenance”, is used for the fed-batch or maintenance fermentation mode with the carbon sources of interest at a flow rate calibrated as a function of the growth of the microorganism.

TABLE 4

| Ingredients in | CM1 | CM2 | CM3 |
|---|---|---|---|
| grams per litre | «inoculum» | «batch» | «maintenance» |
| (NH₄)₂SO₄ | 4.7 | 4.7 | |
| Na₂HPO₄•7H₂O | 12 | 9 | |
| KH₂PO₄ | 2.7 | 2.03 | |
| MgSO₄•7H₂O | 0.8 | 1.03 | |
| Nutrient Broth | 3 | / | |
| Caprylic acid | / | 0.9 | 900 |
| Undecylenic acid | / | 0.1 | 100 |
| Microelement solution | / | 10 | |
| Acrylic acid | / | / | |

| | |
|---|---|
| 2N NaOH | qs pH = 6.8 |
| milliQ water | qs 1000 g |

The composition of the Nutrient Broth, as mass percentages, is 37.5% beef extract and 62.5% peptone. Reference 233000 DIFCO™.

TABLE 5

| Ingredients in grams per litre | Amount | |
|---|---|---|
| FeSO₄•7H₂O | 10.0 | g |
| CaCl₂•2H₂O | 3.0 | g |
| ZnSO₄•7H₂O | 2.2 | g |
| MnSO₄•4H₂O | 0.5 | g |
| H₃BO₃ | 0.3 | g |
| CoCl₂•6H₂O | 0.2 | g |
| Na₂MoO₄•2H₂O | 0.15 | g |
| NiCl₂•6H₂O | 0.02 | g |
| CuSO₄•5H₂O | 1.00 | g |

| | |
|---|---|
| MilliQ water (or 0.5N HCl) | qs 1000 g |

100 mL of preculture are prepared by suspending a cryotube containing 1 mL of the strain with 100 mL of “inoculum” culture medium at a pH adjusted to 6.8 with 2N NaOH in a 250 mL Fernbach flask and are then incubated at 30° C. at 150 rpm for 24 hours. 1.9 L of CM2 “batch” culture medium placed in a presterilized 3 L chemostat are inoculated at O_{D}=0.1 with the 100 mL of preculture. After 4 hours at 30° C. at 850 rpm.

At the end of the introduction, the biomass is isolated by centrifugation and then washed three times with water. The biomass is dried by lyophilization before being extracted with ethyl acetate for 24 hours. The suspension is clarified by filtration on a GE/A filter (Whatman®). The filtrate, composed of PHA dissolved in the ethyl acetate, is concentrated by evaporation and then dried under high vacuum at 40° C. to constant mass.

The PHA may optionally be purified by successive dissolution and precipitation from an ethyl acetate/ethanol 70% methanol system, for example.

The PHA was fully characterized by spectroscopic and spectrometric methods and is in accordance with the expected chemical structure.

### Preparation of Example 1′: PHA Copolymer Bearing a Side Chain R¹ Representing a 5% Unsaturated n-octenyl Group and R² Representing an n-hexyl Group

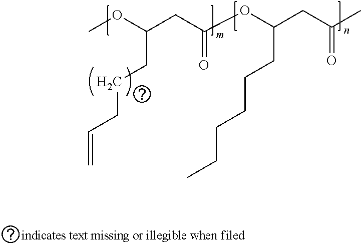

The copolymer of Example 1′ (5% unsaturation and R² chain representing n-hexyl) was prepared according to the procedure described for Example 1, with the same composition of the microelement solution as described in Example 1 and with the following culture medium compositions:

TABLE 6

| Ingredients in | CM1 | CM2 | CM3 |
|---|---|---|---|
| grams per litre | “inoculum” | “batch” | “maintenance” |
| (NH₄)₂SO₄ | 4.7 | 4.7 | |
| Na₂HPO₄•7H₂O | 12 | 9 | |
| KH₂PO₄ | 2.7 | 2.03 | |
| MgSO₄•7H₂O | 0.8 | 1.03 | |
| Nutrient Broth | 3 | / | |
| Nonanoic acid | / | 0.95 | 950 |
| Undecylenic acid | | 0.05 | 50 |
| Microelements Solution | / | 10 | |

| | |
|---|---|
| 2N NaOH | qs pH = 6.8 |
| MilliQ water | qs 1000 g |

The PHA copolymer of Example 1′ was fully characterized by spectroscopic and spectrometric methods and is in accordance with the expected chemical structure, with a degree of unsaturation of 5%.

### Example 1″: PHA Copolymer Bearing a Side Chain R¹ Representing a Linear 10% Unsaturated n-octenyl Group and R² Representing an n-hexyl Group

The copolymer of Example 1″ (10% unsaturation and R² chain representing n-hexyl) was prepared according to the procedure described for Example 1, with the same composition of the microelement solution as described in Example 1 and with the following culture medium compositions:

TABLE 7

| Ingredients in | CM1 | CM2 | CM3 |
|---|---|---|---|
| grams per litre | “inoculum” | “batch” | “maintenance” |
| (NH₄)₂SO₄ | 4.7 | 4.7 | / |
| Na₂HPO₄•7H₂O | 12 | 9 | / |
| KH₂PO₄ | 2.7 | 2.03 | / |
| MgSO₄•7H₂O | 0.8 | 1.03 | / |
| Nutrient Broth | 3 | / | / |
| Nonanoic acid | / | 0.90 | 900 |
| Undecylenic acid | / | 0.1 | 100 |
| Microelements Solution | / | 10 | / |

| | |
|---|---|
| 2N NaOH | qs pH = 6.8 |
| MilliQ water | qs 1000 g |

The PHA was fully characterized by spectroscopic and spectrometric methods and is in accordance with the expected chemical structure.

### Example 1″′: PHA Copolymer Bearing a Side Chain R¹ Representing a Linear 30% Unsaturated n-octenyl Group and R² Representing an n-pentyl Group

The copolymer of Example 1″′ (30% unsaturated and R² chain representing n-pentyl) was prepared according to the procedure described for Example 1, with the same composition of the microelement solution as described in Example 1 and with the following culture medium compositions:

TABLE 8

| Ingredients in | CM1 | CM2 | CM3 |
|---|---|---|---|
| grams per litre | “inoculum” | “batch” | “maintenance” |
| (NH₄)₂SO₄ | 4.7 | 4.7 | / |
| Na₂HPO₄•7H₂O | 12 | 9 | / |
| KH₂PO₄ | 2.7 | 2.03 | / |
| MgSO₄•7H₂O | 0.8 | 1.03 | / |
| Nutrient Broth | 3 | / | / |
| Octanoic acid | / | 0.70 | 700 |
| Undecylenic acid | / | 0.3 | 300 |
| Microelements Solution | / | 10 | / |

| | |
|---|---|
| 2N NaOH | qs pH = 6.8 |
| MilliQ water | qs 1000 g |

The PHA copolymer was fully characterized by spectroscopic and spectrometric methods and is in accordance with the expected chemical structure.

### Example 1″″: PHA Copolymer Bearing a Side Chain R¹ Representing a 2% Unsaturated n-octenyl Group and R² Representing an n-hexyl Group Unsaturated Carried Out in Discontinuous Culture Fed with Two Sources of Carbon in C9 and C11: 1 98/2

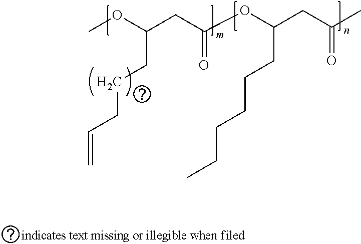

The process for obtaining example 1″″ is adapted from Appl Microbiol Biotechnol 82:657-662 (2009).

“Fed-batch production of unsaturated medium-chain-length polyhydroxyalkanoates with controlled composition by Pseudomonas putida KT2440”.

The microorganism used is Pseudomonas putida KT2440 ATCC®47054™ The culture mode is carried out under axenic conditions in discontinuous growth fed with a maintenance solution containing a mixture of carbon sources at a rate of μ=0.15 h-1 in a 3 L chemostat containing 2.5 L of medium of culture. The flow rate of the maintenance supply pump is proportional to the growth of the microorganism according to formula 1:

$$St = \frac{{X}_{t}}{{Y}_{X / S}} = \frac{{X}_{0}}{{Y}_{X / S}} {e}^{\text{?} t}$$

$$\text{?} \text{indicates text missing or illegible when filed}$$

Formula 1: theoretical equation linking the quantity of biomass and carbon source as a function of time with St=quantity of carbon source required to produce the biomass Xt at time t (g), Y_{X/S}=biomass yield from the carbon source, X₀=initial biomass (g) and μ=desired specific growth rate (h-1)

The system is aerated by an air flow of 0.5 vvm for a dissolved oxygen (DO) setpoint at 30% saturation. The pH is regulated with a 15% of ammonia solution. The temperature of the fermentation medium is regulated at 30° C. The Assembly of the fed batch growth fermentation mode is made according to FIG. 1.

The fermentation medium is regulated in temperature-dissolved oxygen pressure and pH (not shown on the fig.).

The production process is carried out using three distinct culture media. The first culture medium defined CM1 “inoculum” is used for the preparation of the preculture.

The second culture medium defined CM2 “bach” is used for the non-supplied discontinuous growth of the microorganism with the primary carbonaceous sources in the Fernbachs flasks.

The third culture medium defined (CM3 “maintenance”) is used for the discontinuous feeding, or maintenance, of the fermentation with the carbonaceous sources of interest at a rate calibrated according to the growth of the microorganism.

The composition in grams per liter of the three media is described in Table 8a:

TABLE 8a
Composition in grams per liter of culture
media for preculture and maintenance.

| | CM1 | CM2 | CM3 |
|---|---|---|---|
| | «inoculum» | «batch» | «maintenance» |
| (NH₄)₂SO₄ | 4.7 | 4.7 | |
| Na₂HPO₄; 7H₂O | 12 | 9 | |
| KH₂PO₄ | 2.7 | 2.03 | |
| MgSO₄; 7H₂O | 0.8 | 1.03 | |
| Nutrient Broth | 3 | / | |
| Nonanoic acid | / | 0.98 | 980 |
| Undecylenic acid | | 0.02 | 20 |
| Microelement solution | / | 10 | |
| Acrylic acid | / | / | |

| | |
|---|---|
| NaOH 2N | QSP pH = 6.8 |
| MilliQ water | QSP m = 1000 g |

The composition of Nutrient Broth in mass percentage is 37.5% beef extract and 62.5% peptone. Reference 233000 DIFCO™.

The composition of the solution of microelements in grams per liter is described in Table 8b:

TABLE 8b
composition in grams per liter of the solution of microelements.

| | | |
|---|---|---|
| FeSO₄•7H₂O | 10.0 | g |
| CaCl₂•2H₂O | 3.0 | g |
| ZnSO₄•7H₂O | 2.2 | g |
| MnSO₄•4H₂O | 0.5 | g |
| H₃BO₃ | 0.3 | g |
| CoCl₂•6H₂O | 0.2 | g |
| Na₂MoO₄•2H₂O | 0.15 | g |
| NiCl₂•6H₂O | 0.02 | g |
| CuSO₄•5H₂O | 1.00 | g |

| | |
|---|---|
| MilliQ water (or HCl 0.5N) | QSP 1000 g |

100 mL of preculture are prepared by suspending a cryotube containing 1 mL of the strain with 100 mL “inoculum” culture media at pH adjusted to 6.8 with 2N NaOH in a 250 mL Fernbach flask then incubating at 30° C. at 150 rpm for 24 hours. 1.9 L of CM2 “BATCH” culture medium placed in a previously sterilized 3 L chemostat are inoculated at OD=0.1 with the 100 mL of preculture. After 4 hours at 30° C. at 850 rpm, the introduction of the maintenance is carried out by applying the flow rate defined by equation 1. At the end of the introduction, the biomass is isolated by centrifugation then washed three times with some water. The biomass is dried by freeze-drying before being extracted with dichloromethane for 24 hours. The suspension is clarified by filtration on a GF/A filter (Wattman®) the filtrate, composed of PHA in solution in dichloromethane, is concentrated by evaporation then dried under high vacuum at 40° C. until constant mass. The PHA can optionally be purified by solubilization and successive precipitations such as a dichloromethane methanol system for example. The PHA was characterized by gas chromatography equipped with an FID detector. It conforms to the expected chemical structure, with an unsaturation rate of 2%.

### Example 2: Poly(3-hydroxyoctanoate-co-undecenoate) Containing 10% Unsaturations 100% Grafted with thiolactic Acid (Compound of Example 1 Grafted with thiolactic Acid TLA)

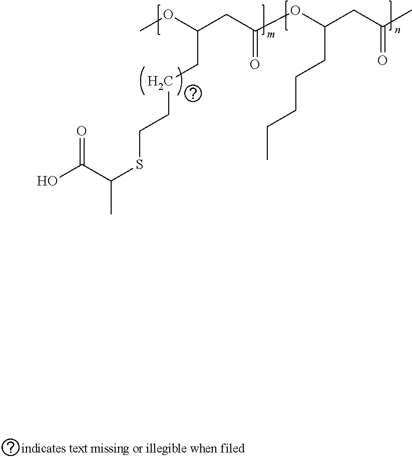

1 g of the compound of Example 1 and 150 mg of thiolactic acid were dissolved in 20 mL of ethyl acetate at room temperature with stirring. 20 mg of 2,2-dimethoxy-2-phenylacetophenone (Irgacure 651) were added to the mixture. The medium was then irradiated under a 100 W UV lamp at 365 nm (reference) and with stirring for at least 10 minutes.

20 mL of the reaction medium were then precipitated from a 200 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 2 was fully characterized by spectroscopic and spectrometric methods and is in accordance with the expected chemical structure.

### Example 3: Poly(3-hydroxyoctanoate-co-undecenoate) Containing 10% Unsaturations 100% Grafted with octanethiol (Compound of Example 1 Grafted with n-octanethiol)

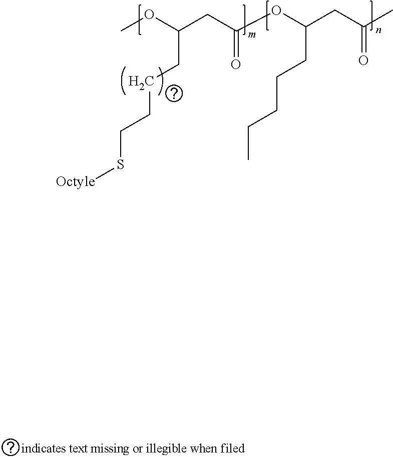

0.5 g of the compound of Example 1 and 125 mg of octanethiol were dissolved in 10 mL of ethyl acetate at room temperature with stirring. 15 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 100 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 3 was fully characterized by spectroscopic and spectrometric methods and is in accordance with the expected chemical structure.

### Example 4: Poly(3-hydroxyoctanoate-co-undecenoate) Containing 10% Unsaturations 75% Grafted with 8-mercapto-1-octanol (Compound of Example 1 Grafted with 8-mercapto-1-octanol)

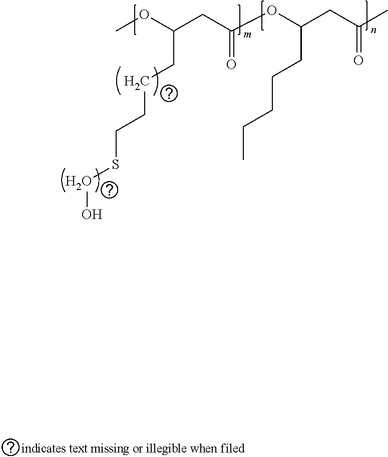

50 mg of the compound of Example 1 and 10 mg of 8-mercapto-1-octanol were dissolved in 5 mL of ethyl acetate at room temperature with stirring. 2 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 50 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 4 was fully characterized by spectroscopic and spectrometric methods and is in accordance with the expected chemical structure. Grafting to 75% or 7.5% of functions in total.

### Example 5: Poly(3-hydroxyoctanoate-co-undecenoate) Containing 10% Unsaturations 32% Grafted with cysteamine (Compound of Example 1 Grafted with cysteamine)

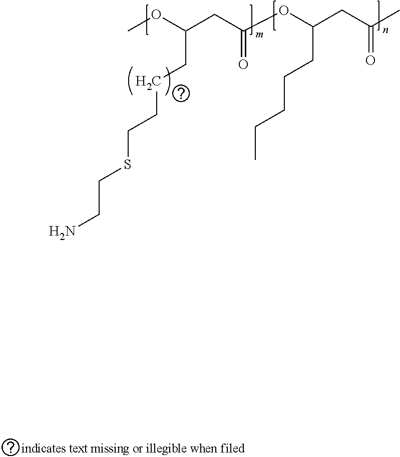

0.5 g of the compound of Example 1 and 54 mg of cysteamine were dissolved in a mixture of 10 mL of dichloromethane and 2 mL of ethanol at room temperature with stirring. 10 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 100 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 5 was fully characterized by spectroscopic and spectrometric methods and is in accordance with the expected chemical structure. Grafting to 32% or 3.2% of functions in total.

### Example 6: Poly(3-hydroxyoctanoate-co-undecenoate) Containing 10% Unsaturations 73% Grafted with cyclohexanethiol (CHT) (Compound of Example 1 Grafted with CHT)

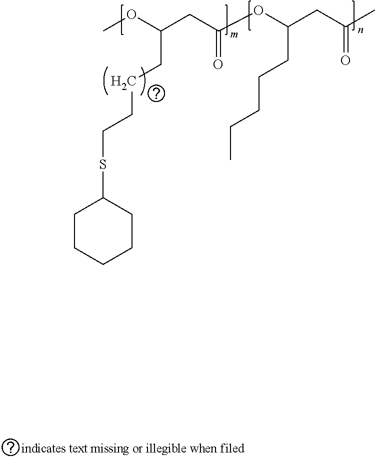

100 mg of the compound of Example 1 and 26 mg of cyclohexanethiol were dissolved in 5 mL of dichloromethane at room temperature with stirring. 5 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 50 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 6 was fully characterized by spectroscopic and spectrometric methods and is in accordance with the expected chemical structure. Grafting to 73% or 7.3% of functions in total.

### Example 7: Poly(3-hydroxyoctanoate-co-undecenoate) Containing 10% Unsaturations 66% Grafted with 2-furanmethanethiol (FT) (Compound of Example 1 Grafted with FT)

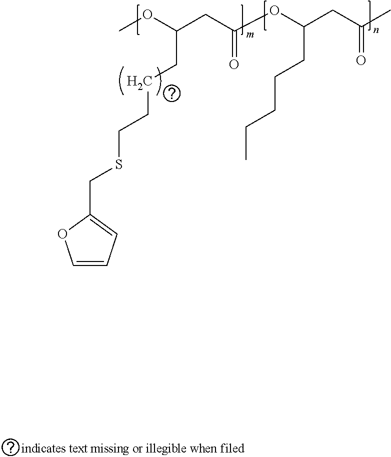

100 mg of the compound of Example 1 and 26 mg of 2-furanmethanethiol were dissolved in 5 mL of dichloromethane at room temperature with stirring. 5 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 50 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 7 was fully characterized by spectroscopic and spectrometric methods and is in accordance with the expected chemical structure. Grafting to 66% or 6.6% of functions in total.

### Example 8: Poly(3-hydroxyoctanoate-co-undecenoate) Containing 10% Unsaturations 70% Grafted with 1-thio-β-D-glucose tetraacetate (Compound of Example 1 Grafted with TGT)

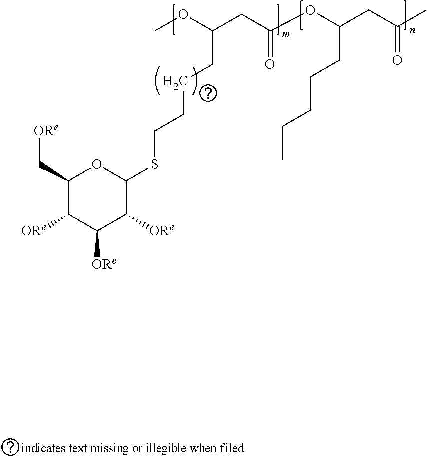

with:
R^{e} representing a *—C(O)—CH₃ group

100 mg of the compound of Example 1 and 26 mg of 1-thio-β-D-glucose tetraacetate were dissolved in 5 mL of dichloromethane at room temperature with stirring. 5 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 50 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 8 was fully characterized by spectroscopic and spectrometric methods and is in accordance with the expected chemical structure. Grafting to 70% or 7% of functions in total.

### Example 9: Poly(3-hydroxyoctanoate-co-undecenoate) Containing 10% Unsaturations 50% Grafted with 2-phenylethanethiol (PT) (Compound of Example 1 Grafted with PT)

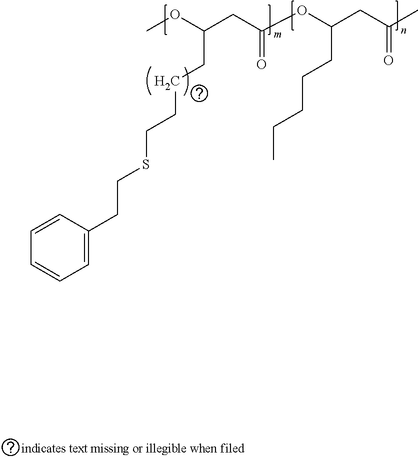

100 mg of the compound of Example 1 and 26 mg of 2-phenylethanethiol were dissolved in 5 mL of dichloromethane at room temperature with stirring. 5 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 50 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 9 was fully characterized by spectroscopic and spectrometric methods and is in accordance with the expected chemical structure. Grafting to 50% or 5% of functions in total.

### Example 10: Poly(3-hydroxyoctanoate-co-undecenoate) Containing 10% Unsaturations 64% Grafted with 4-tert-butylbenzyl mercaptan (TBM) (Compound of Example 1 Grafted with TBM)

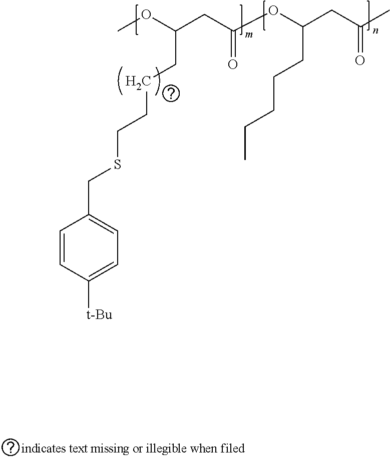

100 mg of the compound of Example 1 and 26 mg of 4-tert-butylbenzyl mercaptan were dissolved in 5 mL of dichloromethane at room temperature with stirring. 5 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 50 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 10 was fully characterized by spectroscopic and spectrometric methods and is in accordance with the expected chemical structure. Grafting to 64% or 6.4% of functions in total.

### Example 11: Poly(3-hydroxynonanoate-co-undecenoate) Containing 10% Unsaturations 100% Grafted with thiolactic Acid

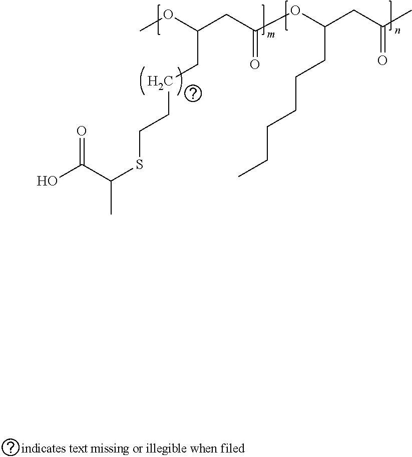

0.1 g of the compound of Example 1″ and 15 mg of thiolactic acid were dissolved in 5 mL of chloroform at room temperature with stirring. 5 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 50 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 11 was fully characterized by spectroscopic and spectrometric methods and is in accordance with the expected chemical structure. Grafting to 100%.

### Example 11′: Functionalization of mcl-PHA with Linear Side Chain R¹ Representing a n-octylenyl Group and R² n-hexyl Unsaturated at 2% of Example 1″″ with thiolactic Acid

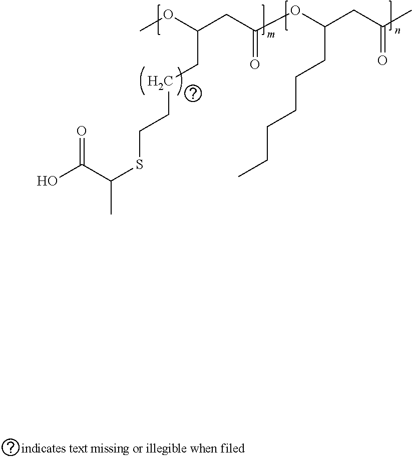

2 g of compound of example 1″″ and 180 mg of thiolactic acid were dissolved in 15 mL of ethyl acetate at room temperature with stirring. 5 mg of 2-Hydroxy-2-methylpropiophenone (HMP) was added to the mixture. The medium was then irradiated under a 100 W UV lamp at 365 nm (reference) and with stirring for at least 10 minutes. The reaction medium thus obtained is poured onto a Teflon plate, then dried under dynamic vacuum at 40° C., to obtain a homogeneous film. The PHA grafted with thiolactic acid was fully characterized by proton NMR. The proton NMR spectrum shows that the characteristic signals of the unsaturations have completely disappeared.

### Example 12: Poly(3-hydroxynonanoate-co-undecenoate) Containing 5% Unsaturations 100% Grafted with octanethiol

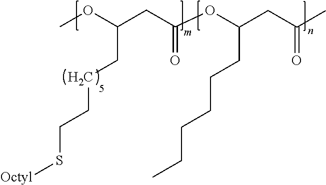

1 g of the PHA copolymer of Example 1′ and 150 mg of octanethiol were dissolved in 15 mL of ethyl acetate at room temperature with stirring. 20 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 500 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 12 was fully characterized by spectroscopic and spectrometric methods and is in accordance with the expected chemical structure. Grafting to 100%.

### Example 13: Poly(3-hydroxynonanoate-co-undecenoate) Containing 5% Unsaturations 100% Epoxidized

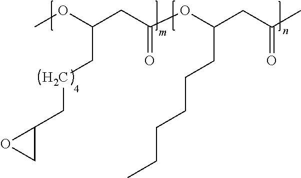

20 g of the PHA copolymer of Example 1′ were dissolved in 80 mL of anhydrous dichloromethane. A suspension of 1.9 g of 77% m-CPBA was prepared with 20 mL of anhydrous dichloromethane and added to the mixture with stirring, at room temperature for at least 120 hours.

The reaction medium was then precipitated from a 500 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The PHA of Example 13 was fully characterized by spectroscopic and spectrometric methods and is in accordance with the expected chemical structure. Epoxidation to 100%.

### Example 14: Poly(3-hydroxynonanoate-co-undecenoate) Containing 10% Unsaturations 100% Epoxidized

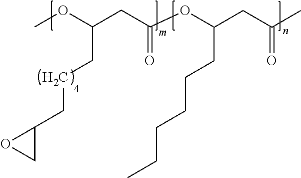

10 g of the PHA copolymer of Example 1″ (10% degree of unsaturation) were dissolved in 40 mL of anhydrous dichloromethane. A suspension of 1.9 g of 77% m-CPBA was prepared with 10 mL of anhydrous dichloromethane and added to the mixture with stirring, at room temperature for at least 120 hours.

The reaction medium was then precipitated from a 500 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The PHA of Example 14 was fully characterized by spectroscopic and spectrometric methods and is in accordance with the expected chemical structure. Epoxidation to 100%.

### Example 15: Poly(3-hydroxyoctanoate-co-undecenoate) Containing 30% Unsaturations 100% Epoxidized

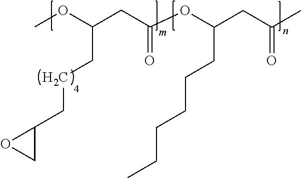

10 g of the PHA copolymer of Example 1″′ (30% degree of unsaturation) were dissolved in 40 mL of anhydrous dichloromethane. A suspension of 6.2 g of 77% m-CPBA was prepared with 10 mL of anhydrous dichloromethane and added to the mixture with stirring, at room temperature for at least 120 hours.

The reaction medium was then precipitated from a 250 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The PHA of Example 15 was fully characterized by spectroscopic and spectrometric methods and is in accordance with the expected chemical structure. Epoxidation to 100%.

### Example 16: Poly(3-hydroxynonanoate-co-undecenoate) Containing 5% Unsaturations 100% Grafted with 4-tert-butylbenzyl mercaptan (TBM) (Compound of Example 1′ Grafted with TBM)

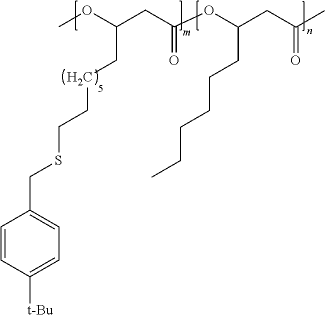

2 g of the PHA copolymer of Example 1′ and 300 mg of 4-tert-butylbenzyl mercaptan were dissolved in 25 mL of ethyl acetate at room temperature with stirring. 25 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 500 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The PHA of Example 16 was fully characterized by spectroscopic and spectrometric methods and is in accordance with the expected chemical structure. Grafting to 100%.

### Example 17: Copolymer of PHA Bearing a Side Chain R¹ Representing an isohexenyl Group and R² Representing an isobutyl Group

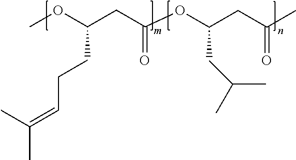

The production process of Example 17 is an adaptation of Applied and Environmental Microbiology, Vol. 60, No. 9. 3245-3254 (1994) “Polyester Biosynthesis Characteristics of Pseudomonas citronellolis Grown on Various Carbon Sources, Including 3-Methyl-Branched Substrate”. Mun Hwan Choi and Sung Chul Yoon. The microorganism used is Pseudomonas citronellolis ATCC® 13674™. The culture method was performed under axenic conditions in unfed batch culture mode in 5 L Fernbach flasks (Corning® ref. 431685) containing 2 of culture medium, shaken at 110 rpm at 30° C. in an orbital incubator (orbit diameter of 2.5 cm).

The production process is performed using two different culture media. The first culture medium, defined CM1 “inoculum”, is used for the preparation of the preculture. The second culture medium, defined CM2 “batch”, is used for unfed batch culture growth of the microorganism with the carbon source of interest in the Fernbach flasks.

TABLE 9

| Ingredients in | CM1 | CM2 |
|---|---|---|
| grams per litre | «inoculum» | «batch» |
| (NH₄)₂SO₄ | / | 0.66 |
| Na₂HPO₄•7H₂O | / | 7.3 |
| KH₂PO4 | / | 2.3 |
| NaHCO₃ | / | 0.3 |
| CaCl₂•2H₂O | / | 0.1 |
| MgSO₄•7H₂O | / | 0.25 |
| Citric acid | / | 1.03 |
| Citronellol | / | 5.5 |
| Microelement solution | / | 1 |
| Nutrient broth | 1.5 | / |
| Yeast extract | 1 | / |

| | |
|---|---|
| 2N NaOH | qs pH = 6.8 |
| milliQ water | qs m = 1000 g |

The composition of the Nutrient Broth, as mass percentages, is 37.5% beef extract and 62.5% peptone. Reference 233000 DIFCO™ BD. The composition of the yeast extract, as a mass percentage, is 100% autolysate of the yeast Saccharomyces cerevisiae. Reference 210933 DIFCO™ BD.

TABLE 10

| Ingredients in grams per litre | Amount | |
|---|---|---|
| FeSO₄•7H₂O | 5.56 | g |
| CaCl₂•2H₂O | 3.0 | g |
| ZnSO₄•7H₂O | 0.58 | g |
| MnCl₂•4H₂O | 3.86 | g |
| H₃BO₃ | 0.0 | g |
| CoCl₂•6H₂O | 5.62 | g |
| Na₂MoO₄•2H₂O | 0.06 | g |
| NiCl₂•6H₂O | 0.04 | g |
| CuSO₄•5H₂O | 0.34 | g |

| | |
|---|---|
| 0.5N HCl | qs 1000 g |

100 mL of preculture are prepared by suspending a cryotube containing 1 mL of the strain with 100 mL of “inoculum” culture medium at a pH adjusted to 6.8 with 2N NaOH in a 250 mL Fernbach flask and then incubated at 30° C. at 150 rpm for 24 hours. 1.9 L of CM2 “batch” culture medium placed in a presterilized 5 L Fernbach flask are inoculated at O_{D}=0.1 with 100 mL of inoculum.

After 70 hours at 30° C. at 110 rpm, the biomass is dried by lyophilization before being extracted with dichloromethane for 24 hours. The suspension is clarified by filtration on a GF/A filter (Whatman®). The filtrate, composed of PHA dissolved in dichloromethane, is concentrated by evaporation and then dried under high vacuum at 40° C. to constant mass.

The PHA may optionally be purified by successive dissolution and precipitation, for instance using a dichloromethane/methanol system.

The PHA copolymer of Example 3 was fully characterized by spectroscopic and spectrometric methods and is in accordance with the expected chemical structure, with: 68 mol % of unit (A) for which R¹=isohexenyl and 32 mol % of unit (B) for which R²=isobutyl.

### Example 18: Copolymer of PHA Bearing a Side Chain R¹ Representing an isohexyl Group and R² Representing an isobutyl Group

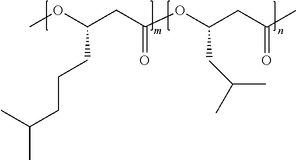

Example 18 is obtained by hydrogenation of the PHA copolymer of Example 17 using an H-Cube Midi® continuous hydrogenator from ThalesNano Technologies.

A solution of 2 g (8.83 mmol) of PHA of Example 17 is prepared with a mixture composed of 100 ml of ethyl acetate (Sigma-Aldrich—CAS: 141-78-6) and 100 mL of methanol (Sigma-Aldrich—CAS: 67-56-1) and is introduced at a flow rate of 3 mL per minute into a hydrogenation cartridge containing the catalyst containing 5% palladium on charcoal (MidiCard ref. DHS 2141; ThalesNano Technologies) maintained at 100° C. under a pressure of 80 bar in the presence of hydrogen in the ThalesNano Technologies H-Cube Midi® system. The reduction of the double bond is monitored by NMR. After six consecutive cycles of reduction, the solution is concentrated by evaporation and then dried under vacuum to constant mass.

The PHA may optionally be purified by successive dissolution and precipitation, for instance using a dichloromethane/methanol system.

The PHA copolymer of Example 18 was fully characterized by spectroscopic and spectrometric methods and is in accordance with the expected chemical structure, with: 68 mol % of unit (A) for which R¹=isohexyl and 32 mol % of unit (B) for which R²=isobutyl.

### Example 19

A polymer was prepared using the microorganism Pseudomonas putida KT2440 ATCC 47054™ and octanoic acid.

The culture method was performed under batch axenic conditions in 5 L Fernbach flasks (Corning® ref. 431685) containing 2 L of culture medium, shaken at 110 rpm at 30° C. in an orbital incubator (orbit diameter of 2.5 cm).

The synthetic process was performed using two different culture media. The first culture medium, defined CM1 “inoculum”, was used for the preparation of the inoculum. The second culture medium, defined CM2 “batch”, was used for unfed batch growth of the microorganism with the octanoic acid in the Fernbach flasks.

The composition in grams per litre of the two media is described in Table 8 below:

TABLE 11

| | CM1 | CM2 |
|---|---|---|
| | «inoculum» | «batch» |
| (NH₄)₂SO₄ | 4.7 | 5.02 |
| Na₂HPO₄•7H₂O | 12 | 2.24 |
| KH₂PO₄ | 2.7 | 0.5 |
| Glucose | 9 | 3.9 |
| MgSO₄•7H₂O | 0.8 | 1.03 |
| Citric acid | 1.6 | 1.03 |
| Nutrient Broth (1) | 1 | / |
| Octanoic acid | / | 3.8 |
| Microelement solution (2) | / | 1.4 |

| | |
|---|---|
| 2NNaOH | qs pH = 6.8 |
| Water | qs 1000 g |

(1) The composition of the Nutrient Broth, as mass percentages, is 37.5% beef extract and 62.5% peptone. Reference 233000 DIFCO ™.
(2) The composition of the microelement solution in grams per litre is described in Table 9 below:

TABLE 12

| | | |
|---|---|---|
| FeSO₄•7H₂O | 10.0 | g |
| CaCl₂•2H₂O | 3.0 | g |
| ZnSO₄•7H₂O | 2.2 | g |
| MnSO₄•4H₂O | 0.5 | g |
| H₃BO₃ | 0.3 | g |
| CoCl₂•6H₂O | 0.2 | g |
| Na₂MoO₄•2H₂O | 0.15 | g |
| NiCl₂•6H₂O | 0.02 | g |
| CuSO₄•5H₂O | 1.00 | g |

| | |
|---|---|
| 0.5N HCl | qs 1000 g |

100 ml of inoculum were prepared by suspending a cryotube containing 1 ml of the strain with 100 ml of “inoculum” culture medium at a pH adjusted to 6.8 with 2N NaOH in a 250 mL Fernbach flask and then incubated at 30° C. at 150 rpm for 24 hours. 1.9 L of CM2 “batch” culture medium placed in a presterilized 5 L Fernbach flask were inoculated at O_{D}=0.1 with 100 mL of inoculum. After 70 hours at 30° C. at 110 rev/min, the biomass was dried by lyophilization before being extracted with dichloromethane for 24 h. The suspension was clarified by filtration on a GF/A filter (Whatman®). The filtrate, containing the copolymer in solution in the dichloromethane, was concentrated by evaporation and then dried under high vacuum at 40° C. to constant weight. The crude polyhydroxyalkanoate was purified by precipitation of a solution of the latter in solution in 10 times its weight of dichloromethane from 10 volumes of the solution of cold methanol. The solid obtained was dried under high vacuum at 40° C. to constant mass.

The molecular weight of the polyhydroxyalkanoate obtained was characterized by size exclusion chromatography, with detection by refractive index.
Eluent: THF
Analytical flow rate: 1 mL/min
Injection: 100 μL
Columns: 1 Agilent PLGel Mixed-D 5 μm column; 300×7.5 mm; 1 Agilent PLGel Mixed-C 5 μm column; 300×7.5 mm; 1 Agilent Oligopore column; 300×7.5 mm at room temperature (25° C.)
Detection: Waters 2487 Dual I Absorbance Detector, Waters 2414 Refractive Index Detector
Integrator: refractive index at 45° C. and 64 mV
Empower (GC Relative molar mass/conventional module)
Empower injection time: 40 min
Standards: High mass/EasiVial PS—H 4 mL polystyrene from Agilent Technologies, Part No. PL2010-0200

The analysis makes it possible to measure the weight-average molecular weight (Mw in g/mol), the number-average molecular weight (Mn in g/mol), the polydispersity index PI (Mw/Mn) and the degree of polymerization DPn.

The monomeric composition of the polyhydroxyalkanoate obtained was defined by gas chromatography equipped with a flame ionization detector. The identification is performed by injection of commercial standards and the monomer composition was determined by a methanolysis and silylation treatment. To determine the monomer composition, 7 mg of the polyhydroxyalkanoate polymer were dissolved in 1.5 mL of chloroform and subjected to methanolysis in the presence of 1.5 mL of an MeOH/HCl solution (17/2, v/v) at 100° C. for 4 hours. The organic phase was then washed with 1 mL of water and then dried over MgSO₄. Silylation of the methyl esters formed was performed by adding 100 μL of BSTFA (N,O-bis(trimethylsilyl)trifluoroacetamide) and 100 μL of pyridine to the methylated sample. The solution was heated at 70° C. for 1 hour and then evaporated to dryness. The sample is then dissolved in 600 μL of dichloromethane and analysed by chromatography under the following conditions:
Hewlett Packard 6890 Series machine
ZB-5 HT stationary phase column from Phenomenex (length: 30 m, diameter: 0.25 mm)
Temperature: isotherm 60° C. to 300° C. in 6 min (heating rate: 10° C./min)
Gas: Helium; flow rate: 0.8 mL/min
Injector: Temperature: 250° C.; 50 ml/min
Flame ionization detector; Temperature: 300° C.
Injection: Volume 1 μL
A copolymer containing 91% by weight of poly(3-hydroxyoctanoate), 6% by weight of poly(3-hydroxyhexanoate) and 3% by weight of poly(3-hydroxybutanoate) was thus obtained.
Mn=68 100 g/mol
Mw=149 100 g/mol
lp=2.2
DPn=531

### Example 20

A polymer was prepared using the microorganism Pseudomonas putida KT2440 ATCC® 47054™, octanoic acid and acrylic acid.

The culture method was performed under continuous axenic conditions at a dilution D=0.25 h⁻¹ in a 3 L chemostat containing 1.1 L of culture medium. The system was aerated with air at a flow of 3 vvm (vvm=volume of air per volume of fermentation medium per minute) for a nominal dissolved oxygen (O_{D}) value at 30% of saturation.

The production process was performed using three different culture media. The first undefined culture medium (CM1) was used for the preparation of the inoculum. The second defined culture medium (CM2) was used for the unfed batch growth of the microorganism in the fermenter. The third defined culture medium (CM3) was used for the feeding, or maintenance, of the continuous fermentation containing octanoic acid and acrylic acid (inhibitor of the β-oxidation pathway).

The CM1 and CM2 media are identical to those described in example 1. The composition in grams per litre of the medium CM3 is described in Table 10 below:

TABLE 13

| | CM3 «continuous» |
|---|---|
| (NH₄)₂SO₄ | 5.02 |
| Na₂HPO₄•7H₂O | 2.24 |
| KH₂PO₄ | 0.5 |
| Glucose | 3 |
| MgSO₄•7H₂O | 1.03 |
| Citric acid | 1.03 |
| Nutrient Broth (1) | / |
| Octanoic acid | 3.8 |
| Microelement solution (2) | 1.4 |
| Acrylic acid | 0.2 |
| 2N NaOH | qs pH = 6.8 |
| Water | qs 1000 g |

100 ml of inoculum were prepared by suspending a cryotube containing 1 ml of the strain with 100 ml of Nutrient Broth at a pH adjusted to 7.0 with 2N NaOH in a 250 ml Fernbach flask and were then incubated at 30° C. at 150 rev/min for 24 h.

The fermenter containing 1 litre of culture medium CM2 at 30° C. was inoculated at an optical density of 0.1 at 630 nm (O_{D 630}=0.1). The system was maintained at 30° C. with shaking at 700±200 rpm and regulated in cascade with oxygenation for about 16 hours and/or the time for the microorganism to be able to reach its growth plateau.

Feeding of the fermenter with the medium CM3 was initiated when the microorganism reached its growth plateau, and withdrawal was then performed so as to maintain the initial mass of fermentation medium. Once the equilibrium state was reached in continuous culture, a fraction of the withdrawn material was centrifuged in order to separate the biomass from the fermentation medium. The biomass was dried by lyophilization and then extracted with dichloromethane for 24 hours. The suspension obtained was clarified by filtration through a GF/A filter (Whatman®). The filtrate obtained, comprising the copolymer dissolved in dichloromethane, was concentrated by evaporation and then dried under high vacuum at 40° C. to constant mass. The crude polyhydroxyalkanoate was purified by precipitation of a solution of the latter in solution in 10 times its weight of dichloromethane from 10 volumes of the solution of cold methanol. The solid obtained was dried under high vacuum at 40° C. to constant weight. A copolymer comprising 96% by weight of poly(3-hydroxyoctanoate), 3% by weight of poly(3-hydroxyhexanoate) and 1% by weight of poly(3-hydroxybutanoate) was thus obtained.
Mn=67 900 g/mol:
Mw=142 000 g/mol:
lp=2.1:
DPn=611

### Example 21: Copolymer of PHA Bearing a Side Chain R¹ Representing an n-hexyl Group and R² Representing an n-butyl Group

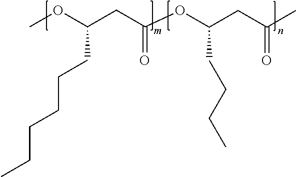

A polymer was prepared using the microorganism Pseudomonas putida KT2440 ATCC® 47054™, nonanoic acid and acrylic acid. The culture method is performed under continuous axenic conditions at a dilution D=0.25 h⁻¹ in a 3 L chemostat containing 1.1 L of culture medium. The system is aerated with a flow of 1 vvm of air for a nominal dissolved oxygen (O_{D}) value at 30% of saturation.

The production process is performed using three different culture media. The first culture medium (CM1) is used for the preparation of the inoculum. The second culture medium (CM2) is used for batch growth of the microorganism in the fermenter. The third culture medium (CM3) is used for the feeding, or maintenance, of the continuous fermentation containing the carbon source of interest and the 3-oxidation pathway inhibitor (acrylic acid). The composition in grams per litre of the three media CM1, CM2 and CM3 is described in Table 14 below:

TABLE 14

| Ingredients in | CM1 | CM2 | CM3 |
|---|---|---|---|
| grams per litre | “inoculum” | “batch” | “continuous” |
| (NH₄)₂SO₄ | 4.7 | 5.02 | 5.02 |
| Na₂HPO₄•7H₂O | 12 | 2.24 | 2.24 |
| KH₂PO₄ | 2.7 | 0.5 | 0.5 |
| Glucose | 9 | 3.9 | 3.9 |
| MgSO₄•7H₂O | 0.8 | 1.03 | 1.03 |
| Citric acid | 1.6 | 1.03 | 1.03 |
| Nutrient Broth | 1 | / | / |
| Nonanoic acid | / | / | 3.8 |
| Microelement Solution | / | 1.4 | 1.4 |
| Acrylic acid | / | / | 0.2 |

| | |
|---|---|
| 2N NaOH | qs pH = 6.8 |
| MilliQ water | qs m = 1000 g |

The composition of the Nutrient Broth, as mass percentages, is 37.5% beef extract and 62.5% peptone. Reference 233000 DIFCO™.

The composition of the microelement solution in grams per litre is described in Table 15 below.

TABLE 15

| | | |
|---|---|---|
| FeSO₄•7H₂O | 10.0 | g |
| CaCl₂•2H₂O | 3.0 | g |
| ZnSO₄•7H₂O | 2.2 | g |
| MnSO₄•4H₂O | 0.5 | g |
| H₃BO₃ | 0.3 | g |
| CoCl₂•6H₂O | 0.2 | g |
| Na₂MoO₄•2H₂O | 0.15 | g |
| NiCl₂•6H₂O | 0.02 | g |
| CuSO₄•5H₂O | 1.00 | g |

| | |
|---|---|
| MilliQ water | qs 1000 g |

100 mL of inoculum are prepared by suspending a cryotube containing 1 mL of the strain at O_{D}=10 with 100 mL of CM1 “inoculum” at a pH preadjusted to 7.0 with 2N NaOH in a 500 mL Fernbach flask and are then incubated at 30° C. at 150 rpm for 24 hours.

The 3 L fermenter containing 1 litre of CM2 “batch” culture medium at 30° C. is inoculated at an optical density of 0.1 at 600 nm (O_{D 600}=0.1). The system is maintained at 30° C. with shaking at 700±200 rpm and regulated in cascade with oxygenation for about 16 hours and/or the time for the microorganism to be able to reach its growth plateau.

Feeding of the fermenter with the CM3 “continuous” medium is initiated when the microorganism has reached its growth plateau, and withdrawal is then performed so as to maintain the initial mass of fermentation medium. Once the equilibrium state is reached in continuous culturing, a fraction of the withdrawn material is centrifuged so as to separate the biomass from the fermentation medium. The biomass is dried by lyophilization and is then extracted with dichloromethane for 24 hours. The suspension is clarified by filtration on a GF/A filter (Whatman®). The filtrate, composed of PHA dissolved in dichloromethane, is concentrated by evaporation and then dried under high vacuum at 40° C. to constant mass.

The molecular weight of the polyhydroxyalkanoate obtained was characterized by size exclusion chromatography, with detection by refractive index.
Eluent: THF
Analytical flow rate: 1 mL/min
Injection: 100 μL
Columns: 1 Agilent PLGel Mixed-D 5 μm column; 300×7.5 mm; 1 Agilent PLGel Mixed-C 5 μm column; 300×7.5 mm; 1 Agilent Oligopore column; 300×7.5 mm
at room temperature (25° C.)
Detection: Waters 2487 Dual I Absorbance Detector, Waters 2414 Refractive Index Detector
Integrator: refractive index at 45° C. and 64 mV
Empower (GC Relative molar mass/conventional module)
Empower injection time: 40 min
Standards: High mass/EasiVial PS—H 4 mL polystyrene from Agilent Technologies, Part No. PL2010-0200

The analysis makes it possible to measure the weight-average molecular weight (Mw in g/mol), the number-average molecular weight (Mn in g/mol), the polydispersity index PI (Mw/Mn) and the degree of polymerization DPn.

The monomeric composition of the polyhydroxyalkanoate obtained was defined by gas chromatography equipped with a flame ionization detector.

The identification is performed by injection of commercial standards and the monomer composition was determined by a methanolysis and silylation treatment.

To determine the monomer composition, 7 mg of the polyhydroxyalkanoate polymer were dissolved in 1.5 mL of chloroform and subjected to methanolysis in the presence of 1.5 mL of an MeOH/HCl solution (17/2, v/v) at 100° C. for 4 hours. The organic phase was then washed with 1 mL of water and then dried over MgSO₄. The silylation of the methyl esters formed was carried out by adding 100 μl of BSTFA (N,O-bis(trimethylsilyl)trifluoroacetamide) and 100 μl of pyridine to the methylated sample. The solution was heated at 70° C. for 1 hour and then evaporated to dryness. The sample is then dissolved in 600 μL of dichloromethane and analysed by chromatography under the following conditions:
Hewlett Packard 6890 Series machine
ZB-5 HT stationary phase column from Phenomenex (length: 30 m, diameter: 0.25 mm)
Temperature: isotherm 60° C. to 300° C. in 6 min (heating rate: 10° C./min)
Gas: Helium; flow rate: 0.8 ml/min
Injector: Temperature: 250° C.; 50 ml/min
Flame ionization detector; Temperature: 300° C.
Injection: Volume 1 μL

A copolymer comprising 86% by weight of poly(3-hydroxynonanoate), 9% by weight of poly(3-hydroxyheptanoate) and 5% by weight of poly(3-hydroxypentanoate) was thus obtained.
Mn=65 900 g/mol
Mw=143 600 g/mol
lp=2.2
DPn=531

### Example 22

A polymer was prepared according to the procedure of example 19 using nonanoic acid (instead of octanoic acid) and without acrylic acid.

A copolymer comprising 68% by weight of poly(3-hydroxynonanoate), 27% by weight of poly(3-hydroxyheptanoate) and 5% by weight of poly(3-hydroxypentanoate) was thus obtained.
Mn=55 800 g/mol
Mw=124 500 g/mol
lp=2.2
DPn=469

### Example 23

A polymer was prepared according to the procedure of example 19 using dodecanoic acid (instead of octanoic acid).

A copolymer comprising 44% by weight of poly(3-hydroxydodecanoate), 38% by weight of poly(3-hydroxydecanoate) and 18% by weight of poly(3-hydroxyoctanoate) was thus obtained.
Mn=67 400 g/mol
Mw=129 800 g/mol
Slp=1.9
DPn=484

### Example 24: Copolymer of PHA Bearing a Side Chain R¹ Representing an n-pentyl Group and R² Representing an n-propyl Group

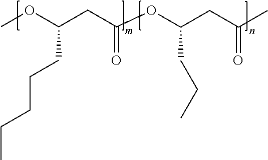

The production process of Example 24 is an adaptation of the article Biomacromolecules 2012, 13, 2926-2932: “Biosynthesis and Properties of Medium-Chain-Length Polyhydroxyalkanoates with Enriched Content of the Dominant Monomer”

The microorganism used is Pseudomonas putida ATCC® 47054™

The culture method is performed under continuous axenic conditions at a dilution D=0.25 h⁻¹ in a 3 L chemostat containing 1.1 L of culture medium.

The system is aerated with a flow of 3 vvm of air for a nominal dissolved oxygen (O_{D}) value at 30% of saturation.

### Assembly:

See FIG. 2

The production process is performed using three different culture media.

The first undefined culture medium (CM1) is used for the preparation of the inoculum.

The second defined culture medium (CM2) is used for batch growth of the microorganism in the fermenter.

The third defined culture medium (CM3) is used for the feeding, or maintenance, of the continuous fermentation containing the carbon source of interest and the β-oxidation pathway inhibitor.

The composition in grams per litre of the three media is described in Table 16. Composition in grams per litre of the culture media for the inoculum and for maintenance.

TABLE 16

| | CM1 | CM2 | CM3 |
|---|---|---|---|
| | «inoculum» | «batch» | «continuous» |
| (NH4)₂SO4 | 4.7 | 5.02 | 5.02 |
| Na₂HPO4•7H2O | 12 | 2.24 | 2.24 |
| KH₂PO4 | 2.7 | 0.5 | 0.5 |
| Glucose | 9 | 3.9 | 3 |
| MgSO4•7H2O | 0.8 | 1.03 | 1.03 |
| Citric acid | 1.6 | 1.03 | 1.03 |
| Nutrient Broth | 1 | / | / |
| Octanoic acid | / | / | 3.8 |
| Microelement solution | / | 1.4 | 1.4 |
| Acrylic acid | / | / | 0.2 |

| | |
|---|---|
| 2N NaOH | qs pH = 8.8 |
| milliQ water | qs m = 1000 g |

The composition of the Nutrient Broth, as mass percentages, is 37.5% beef extract and 62.5% peptone. Reference 233000 DIFCO™.

The composition of the microelement solution in grams per litre is described in Table 17: composition in grams per litre of the microelement solution

TABLE 17

| | | |
|---|---|---|
| FeSO4•7H2O | 10.0 | g |
| CaCl2•2H2O | 3.0 | g |
| ZnSO4•7H2O | 2.2 | g |
| MnSO4•4H2O | 0.5 | g |
| H3BO3 | 0.3 | g |
| CoCl2•6H2O | 0.2 | g |
| Na2MoO4•2H2O | 0.15 | g |
| NiCl2•6H2O | 0.02 | g |
| CuSO4•5H2O | 1.00 | g |

| | |
|---|---|
| MilliQ water | QSP 1000 g |

100 mL of inoculum are prepared by suspending a cryotube containing 1 mL of the strain with 100 mL of Nutrient Broth at a pH adjusted to 7.0 with 2N NaOH in a 250 mL Fernbach flask and are then incubated at 30° C. at 150 rpm for 24 hours.

The fermenter containing 1 litre of culture medium CM2 at 30° C. was inoculated at an optical density of 0.1 at 630 nm (O_{D 630}=0.1). The system is maintained at 30° C. with shaking at 700 rpm±200 rpm and regulated in cascade with oxygenation for about 16 hours and/or the time for the microorganism to be able to reach its growth plateau.

Feeding of the fermenter with the medium CM3 is initiated when the microorganism has reached its growth plateau, and withdrawal is then performed so as to maintain the initial mass of fermentation medium. Once the equilibrium state is reached in continuous culturing, a fraction of the withdrawn material is centrifuged so as to separate the biomass from the fermentation medium. The biomass is dried by lyophilization and is then extracted with dichloromethane for 24 hours. The suspension is clarified by filtration on a GF/A filter (Whatman®). The filtrate, composed of PHA dissolved in dichloromethane, is concentrated by evaporation and then dried under high vacuum at 40° C. to constant mass.

The PHA may optionally be purified by successive dissolution and precipitation, for instance using a dichloromethane/methanol system.

The PHA copolymer of Example 24 was fully characterized by spectrometric and spectroscopic methods. By gas chromatography equipped with an FID detector, it is seen that the copolymer contains 96% of radical R¹=n-pentyl and 4% of radical R²=n-propyl.

### Example 25: PHA Bearing a Side Chain R¹ Representing a Linear 5% Unsaturated 8-bromo-n-octanoyl Group and R² Representing an n-hexyl Group

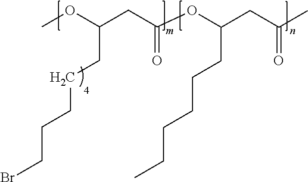

The process for synthesizing the compound of Example 1 is adapted from the article: Fed-batch production of unsaturated medium-chain-length polyhydroxyalkanoates with controlled composition by Pseudomonas putida KT2440, Z. Sun, J. A. Ramsay, M. Guay, B. A. Ramsay, Applied Microbiology Biotechnology, 82. 657-662, 2009.

The microorganism used is Pseudomonas putida KT2440 ATCC® 47054™. The culture method is performed under fed-batch growth axenic conditions with a maintenance solution containing a mixture of carbon source at a rate μ=0.15 h¹ in a 3 L chemostat containing 2.5 L of culture medium.

The system is aerated with a flow of 0.5 vvm of air for a nominal dissolved oxygen (O_{D}) value at 30% of saturation. The pH is regulated with a solution composed of ammonia and glucose at 15% and 40% final mass, respectively. The temperature of the fermentation medium is regulated at 30° C.

Equipment for the fed-batch growth fermentation mode:

The fermentation medium is regulated in terms of temperature-pressure of dissolved oxygen and pH (not shown).

The production process is performed using three different culture media. The first culture medium, defined CM1 “inoculum”, is used for the preparation of the preculture. The second culture medium, defined CM2 “batch”, is used for unfed batch growth of the microorganism with the primary carbon sources in the Fernbach flasks. The third culture medium, defined CM3 “maintenance”, is used for the fed-batch or maintenance fermentation mode with the carbon sources of interest at a flow rate calibrated as a function of the growth of the microorganism.

TABLE 18

| Ingredients in | CM1 | CM2 | CM3 |
|---|---|---|---|
| grams per litre | «inoculum» | «batch» | «maintenance» |
| (NH₄)₂SO₄ | 4.7 | 4.7 | |
| Na₂HPO₄•7H₂O | 12 | 9 | |
| KH₂PO₄ | 2.7 | 2.03 | |
| MgSO₄•7H₂O | 0.8 | 1.03 | |
| Nutrient Broth | 3 | / | |
| Nonanoic acid | / | 1 | 923 |
| 11-Bromoundecanoic acid | / | 0 | 77 |
| Microelement solution | / | 10 | |

| | |
|---|---|
| 2N NaOH | qs pH = 6.8 |
| milliQ water | qs 1000 g |

The composition of the Nutrient Broth, as mass percentages, is 37.5% beef extract and 62.5% peptone. Reference 233000 DIFCO™.

TABLE 19

| Ingredients in grams per litre | Amount | |
|---|---|---|
| FeSO₄•7H₂O | 10.0 | g |
| CaCl₂•2H₂O | 3.0 | g |
| ZnSO₄•7H₂O | 2.2 | g |
| MnSO₄•4H₂O | 0.5 | g |
| H₃BO₃ | 0.3 | g |
| CoCl₂•6H₂O | 0.2 | g |
| Na₂MoO₄•2H₂O | 0.15 | g |
| NiCl₂•6H₂O | 0.02 | g |
| CuSO₄•5H₂O | 1.00 | g |

| | |
|---|---|
| MilliQ water (or 0.5N HCl) | qs 1000 g |

100 mL of preculture are prepared by suspending a cryotube containing 1 mL of the strain with 100 mL of “inoculum” culture medium at a pH adjusted to 6.8 with 2N NaOH in a 250 mL Fernbach flask and then incubated at 30° C. at 150 rpm for 24 hours. 1.9 L of CM2 “batch” culture medium placed in a presterilized 3 L chemostat are inoculated at O_{D}=0.1 with 100 mL of preculture. After 4 hours at 30° C. at 850 rpm, introduction of the maintenance culture medium is performed, applying the flow rate defined by equation 1.

At the end of the introduction, the biomass is isolated by centrifugation and then washed three times with water. The biomass is dried by lyophilization before being extracted with ethyl acetate for 24 hours. The suspension is clarified by filtration on a GE/A filter (Whatman®). The filtrate, composed of PHA dissolved in the ethyl acetate, is concentrated by evaporation and then dried under high vacuum at 40° C. to constant mass.

The PHA may optionally be purified by dissolution and successive precipitations from an ethyl acetate/70% ethanol methanol system, for example.

The PHA was fully characterized by spectroscopic and spectrometric methods and is in accordance with the expected chemical structure: 95 mol % of unit (B) for which R²=n hexyl (71%) and n-butyl (24%) and 5 mol % of unit (A) for which R¹=8-bromo-n-octanyl (5.9%) and 6-bromo-n-hexyl (0.2%).

The compounds of Examples 1 to 25 may be mixed with one or more resins b) as defined previously, preferably chosen from j) elemi resins, k) frankincense, and t) rosins as defined previously; preferably in the presence of a liquid fatty substance c) such as isododecane and/or water e). The mixing of the PHA(s) a) with the resin(s) b) may be performed at room temperature, with stirring, preferably in the presence of a liquid fatty substance c) and optionally of organic solvent(s) other than d) as defined previously. According to one variant, water e) is added to the mixture of a), b) and c) and one or more organic solvents other than c) and d) as defined previously are then optionally added. According to another variant, a) and b) are in dispersion in water and then

### Examples 26 to 31

Compositions 26 (comparative) and 27 to 31 (invention) described in Table 17 below were prepared:

TABLE 17

| | Ex. | Ex. | Ex. | Ex. | Ex. | Ex. |
|---|---|---|---|---|---|---|
| Ingredients | 26 | 27 | 28 | 29 | 30 | 31 |
| PHA of Example 21 | 20 | 20 | 20 | 20 | 14 | 20 |
| Resin extracted from | — | 5 | — | — | — | — |
| candelilla wax | | | | | | |
| Resin from protium | — | — | 5 | — | — | — |
| heptaphyllum | | | | | | |
| (PROTIUM | | | | | | |
| HEPTAPHYLLUM | | | | | | |
| RESIN) | | | | | | |
| Glycerol ester of gum | — | — | — | 5 | — | — |
| rosin/colophony | | | | | | |
| (GLYCEROL ESTER | | | | | | |
| OF HIGHLY | | | | | | |
| STABILIZED | | | | | | |
| GUM ROSIN, | | | | | | |
| DEODORIZED) | | | | | | |
| Methylated and | — | — | — | — | 6 | — |
| hydrogenated rosinate/ | | | | | | |
| colophony (METHYL | | | | | | |
| HYDROGENATED | | | | | | |
| ROSINATE) | | | | | | |
| Kahlwax 6721: resin of | — | — | — | — | — | 5 |
| Shorea robusta and | | | | | | |
| beeswax | | | | | | |
| Isododecane | 77.5 | 30 | 30 | 30 | 77.5 | 77.5 |
| Ethanol | 2.5 | 45 | 45 | 45 | 2.5 | 2.5 |

### Performance Evaluations

### Wear Resistance

### Description of the Test:

The first step in this test consists in making a deposit. The deposits are prepared on a Byko Chart Lenata contrast card and left to dry for 24 hours at 25° C. and 45% RH. The final thickness of the deposit is 30 μm.

A wear resistance test is performed on this dry deposit. A hydrophilic steel ball is used as a friction device. The load or normal force applied is 1N, and the displacement speed is 50 mm·s⁻¹. On each film are defined tracks on which the friction device makes multiple passes. In the case of wear measurements, permanent contact is maintained during the to and fro trips of the ball on the deposit. The number of passes is increased for each track. The wear resistance is quantified as the minimum number of passes to completely wear out the deposit.

In the case of this study, the number of passes per track are, respectively, 10, 30, 50, 100, 200 and 300 passes.

Each measurement was repeated five times.

The results of the wear resistance tests are quantified as described in the table below:

TABLE 18

| Wear resistance | Evaluations |
|---|---|
| 0-10 passes of the ball | −− |
| 10-30 passes of the ball | − |
| 30-50 passes of the ball | + |
| 50-100 passes of the ball | ++ |
| 100-200 passes of the ball | +++ |
| 200-300 passes of the ball | ++++ |
| >300 passes of the ball | +++++ |

TABLE 19

| Examples | Evaluations |
|---|---|
| 26 (comparative) | −− |
| 27 | +++++ |
| 28 | +++++ |
| 29 | ++++ |
| 30 | +++ |
| 31 | ++ |

### Resistance to Water

### Description of the Test:

On the same 30 μm dry deposit made for the wear test, the sensitivity to stressors is evaluated after depositing a drop of stressor (20 μl for water) on the surface of the deposit. The evaluations are made after 1 hour of contact between the stressor and the deposit. The level of sensitivity to stressors is noted as follows.

| Level of | |
|---|---|
| sensitivity | Appearance of the deposit |
| 1 - dissolved | the deposit is dissolved and the support is visible |
| 2 - tacky | the deposit is present but sticks to the finger |
| 3 - marked | the deposit is present and, after wiping off the |
| | drop of water, the imprint of the drop is visible |
| 4 - not | the deposit remains intact after wiping off the |
| dissolved | drop of water |

It is seen that all the compositions of the invention (Ex. 27 to 31) are highly resistant to water since the film remained intact.

### Examples 32 to 40

Compositions 32 to 40 (invention) described in Tables 20 and 21 below were prepared:

TABLE 20

| | Ex. | Ex. | Ex. | Ex. | Ex. | Ex. |
|---|---|---|---|---|---|---|
| | 32 | 33 | 34 | 35 | 36 | 37 |
| PHA of example 21 PHN | 20 | 16 | 12.5 | 20 | 16 | |
| PHA of example 1″″ | | | | | | 10 |
| Candellila resin* | 5 | 9 | 12.5 | | | 1 |
| Protium | | | | 5 | 9 | |
| heptaphyllum resin | | | | | | |
| (White breu)* | | | | | | |
| Isododecane** | 45 | 45 | 45 | 45 | 45 | |
| Parleam** | | | | | | 0.2 |
| Cetiol UT** | | | | | | 88.8 |
| Ethanol | 30 | 30 | 30 | 30 | 30 | |

TABLE 21

| | Ex. 38 | Ex. 39 | Ex. 40 |
|---|---|---|---|
| PHA of example 11′ | 23 | | |
| PHA of example 12 | | 10 | |
| PHA of example 25 | | | 5 |
| Candellila resin* | | 0.5 | 10 |
| Protium heptaphyllum resin (White breu)* | 3 | | |
| Isododecane** | 70 | | 82.5 |
| Isono. Isono**. | | 0.1 | |
| Squalane** | 4 | | |
| Cetiol UT** | | 89.4 | |
| Ethanol | | | 2.5 |

TABLE 22

| | | Commercial | |
|---|---|---|---|
| Resins* | Supplier | name | INCI name |
| Candellila | JAPAN | CANDELILLA | EUPHORBIA |
| resin | NATURAL | RESIN E-1 | CERIFERA |
| | PRODUCTS | | (CANDELILLA) |
| | | | WAX EXTRACT |
| Protium | CITROLEO | CITROBREU | PROTIUM |
| heptaphyllum | | | HEPTAPHYLLUM |
| resin | | | RESIN |
| (White breu) | | | |

TABLE 23

| Oils** | Supplier | Commercial name | INCI name |
|---|---|---|---|
| Emogreen L15 | SEPPIC | EMOGREEN L15 | C15-19 ALKANE |
| ISONONANOATE | OLEON | RADIA 7710 | ISONONYL |
| D'ISONONYLE | | | ISONONANOATE |
| Cetiol UT | BASF | CETIOL UT | UNDECANE (and) |
| | | | TRIDECANE |
| Isododecane | INEOS | ISODODECANE | ISODODECANE |

### Performance Evaluations

### Wear Resistance

The test was the same as the one described above.

Each measurement was repeated five times. The results of the wear resistance tests are quantified as described in the table 24 below:

### Results:

TABLE 24
Results:

| Exemples | Wear resistance evaluations |
|---|---|
| Example 32 | + |
| Example 33 | ++++ |
| Example 34 | ++++ |
| Example 35 | + |
| Exemple 36 | ++++ |
| Example 37 | +++ |
| Example 39 | ++ |
| Example 40 | + |

It is seen that the compositions according to the invention are significantly more resistant than the reference composition not including any resins.

### Resistance to Water

The test was the same as the one described above.

It is seen that the compositions of the invention (Ex. 32 to 40) remain very water-resistant. The presence of several any resins has no impact on the water resistance of the films obtained

1. Composition, notably a cosmetic composition, comprising:
a) one or more polyhydroxyalkanoate (PHA) copolymers comprising at least two different repeating polymer units chosen from the units (A) and (B) below, and also the optical or geometrical isomers thereof, the organic or mineral acid or base salts thereof, and the solvates thereof such as hydrates:
—[—O—CH(R¹)—CH₂—C(O)—]— unit (A)
—[—O—CH(R²)—CH₂—C(O)—]— unit (B)
in which polymer units (A) and (B):
R¹ represents a saturated or unsaturated, linear or branched, non-cyclic hydrocarbon-based chain, or a saturated or unsaturated, aromatic or non-aromatic cyclic hydrocarbon-based chain, comprising from 5 to 28 carbon atoms; preferably, the hydrocarbon-based chain is chosen from i) linear or branched (C₅-C₂₈)alkyl, ii) linear or branched (C₅-C₂₈)alkenyl, iii) linear or branched (C₅-C₂₈)alkynyl; preferably, the hydrocarbon-based group is linear;
said hydrocarbon-based chain being:
optionally substituted with one or more atoms or groups chosen from: a) halogen such as chlorine or bromine, b) hydroxyl, c) thiol, d) (di)(C₁-C₄)(alkyl)amino, e) (thio)carboxy, f) (thio)carboxamide —C(O)—N(Rₐ)₂ or C(S)—N(Rₐ)₂, g) cyano, h) iso(thio)cyanate, i) (hetero)aryl such as phenyl or furyl, and j) (hetero)cycloalkyl such as anhydride, epoxide or dithiolane, k) cosmetic active agent; l) R—X with R representing a group chosen from α) cycloalkyl such as cyclohexyl, β) heterocycloalkyl such as sugar, preferably monosaccharide such as glucose, γ) (hetero)aryl such as phenyl, δ) cosmetic active agent, m) thiosulfate, and X representing a′) O, S, N(Rₐ) or Si(R_{b})(Rₑ), b′) S(O)ᵣ, or (thio)carbonyl, c′) or combinations of a′) with b′) such as (thio)ester, (thio)amide, (thio)urea or sulfonamide; Rₐ representing a hydrogen atom, or a (C₁-C₄)alkyl group or an aryl(C₁-C₄)alkyl group such as benzyl; preferably, Rₐ represents a hydrogen atom; R_{b} and R_{c}, which may be identical or different, represent a (C₁-C₄)alkyl or (C₁-C₄)alkoxy group, particularly only one substituent; preferably chosen from b) halogen, and j) such as epoxide; and/or
optionally interrupted with one or more a′) heteroatoms such as O, S, N(Rₐ) and Si(R_{b})(R_{c}), b′) S(O)ᵣ, (thio)carbonyl, c′) or combinations of a′) with b′) such as (thio)ester, (thio)amide, (thio)urea or sulfonamide with r being equal to 1 or 2, Rₐ being as defined previously; preferably, Rₐ represents a hydrogen atom, R_{b} and R_{c} being as defined previously;
R² represents a cyclic or non-cyclic, linear or branched, saturated or unsaturated hydrocarbon-based group comprising from 3 to 30 carbon atoms optionally substituted with one or more atoms or groups a) to m) and/or optionally interrupted with one or more heteroatoms or groups a′) to c′) as defined for R¹; in particular chosen from linear or branched (C₃-C₂₈)alkyl and linear or branched (C₃-C₂₈)alkenyl, in particular a linear hydrocarbon-based group, more particularly (C₄-C₂₀)alkyl or (C₄-C₂₀)alkenyl; preferably, the hydrocarbon-based group has a carbon number corresponding to the number of carbon atoms of the radical R¹ from which at least one carbon atom is subtracted, preferably corresponding to the number of carbon atoms of the radical R¹ from which two carbon atoms are subtracted;
b) one or more natural resins;
c) optionally one or more fatty substances, which are preferably liquid at 25° C. and at atmospheric pressure;
d) optionally, one or more organic solvents other than c);
e) optionally water;
it being understood that:
(A) is different from (B) and
preferably, the composition contains ingredients c)+d).

2. Composition according to the preceding claim, comprising:
a) one or more polyhydroxyalkanoate (PHA) copolymers comprising the repeating unit chosen from those of formula (I), and also the optical or geometrical isomers thereof, the organic or mineral acid or base salts thereof, and the solvates thereof such as hydrates: 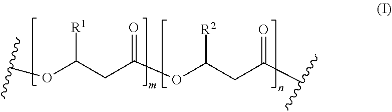
in which formula (I):
R¹ and R² are as defined in claim 1;
m and n are integers greater than or equal to 1; preferably, the sum n+m is inclusively between 450 and 1400;
preferably, m>n when R¹ and R² represent an unsubstituted and uninterrupted alkyl group—more preferentially, when R¹ and R² are linear alkyl, then R¹ is a C₅-C₁₃ alkyl group; and R² represents a linear alkyl group with a carbon number corresponding to the carbon number of R¹ from which two carbon atoms are subtracted; and
preferably, m<n when R¹ represents a substituted and/or interrupted alkyl group, an optionally substituted and/or interrupted alkenyl group or an optionally substituted and/or interrupted alkynyl group, and R² represents an alkyl group.

3. Composition according to any one of the preceding claims, in which the PHA copolymer(s) a) comprise three different repeating polymer units (A), (B) and (C), and preferably consist of three different polymer units (A), (B) and (C) below, and also the optical or geometrical isomers thereof and the solvates thereof such as hydrates:
—[—O—CH(R¹)—CH₂—C(O)—]— unit (A)
—[—O—CH(R²)—CH₂—C(O)—]— unit (B)
—[—O—CH(R³)—CH₂—C(O)—]— unit (C)
in which polymer units (A), (B) and (C):
R¹ and R² are as defined in claim 1;
R³ represents a saturated or unsaturated, linear or branched, cyclic or non-cyclic, hydrocarbon-based group comprising from 1 to 30 carbon atoms, optionally substituted with one or more atoms or groups a) to m) and/or optionally interrupted with one or more heteroatoms or groups a′) to c′) as defined for R¹ in claim 1; it in particular represents a hydrocarbon-based group chosen from linear or branched (C₁-C₂₈)alkyl, and linear or branched (C₂-C₂₈)alkenyl, in particular a linear hydrocarbon-based group, more particularly (C₄-C₂₀)alkenyl; preferably, the hydrocarbon-based group has a carbon number corresponding to the number of carbon atoms in the radical R¹, or else corresponding to the number of carbon atoms in the radical R¹ minus at least three carbon atoms, preferably corresponding to the number of carbon atoms in the radical R¹ minus four carbon atoms; and
it being understood that:
(A) is different from (B) and (C), (B) is different from (A) and (C), and (C) is different from (A) and (B); and
preferably, when R¹, R² and R³ represent an unsubstituted and uninterrupted alkyl group, the molar percentage of units (A) is greater than the molar percentage of units (B), and greater than the molar percentage of units (C)—more preferentially, when R¹, R² and R³ are linear alkyl, then R¹ is a C₅-C₁₃ alkyl group; and R² represents a linear alkyl group with a carbon number corresponding to the carbon number of R¹ from which two carbon atoms are subtracted, and R³ represents a linear alkyl group with a carbon number corresponding to the carbon number of R¹ from which four carbon atoms are subtracted; and
preferably, when R¹ represents a substituted and/or interrupted alkyl, optionally substituted and/or optionally interrupted alkenyl or optionally substituted and/or optionally interrupted alkynyl group, then the molar percentage of units (A) is less than the molar percentage of units (B) and less than the molar percentage of units (C) notably if R² represents an alkyl group and/or R³ represents an alkyl group;
more preferentially, the PHA copolymer(s) a) contain the repeating unit of formula (II), and also the optical or geometrical isomers thereof, the organic or mineral acid or base salts thereof, and the solvates thereof such as hydrates: 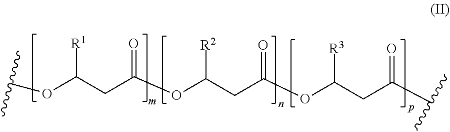
in which formula (II):
R¹, R² and R³ are as defined previously;
m, n and p are integers greater than or equal to 1; preferably, the sum n+m+p is inclusively between 450 and 1400;
preferably, m>n+p when R¹, R² and R³ represent an unsubstituted and uninterrupted alkyl group—more preferentially, when R¹, R² and R³ are linear alkyl, then R¹ is a C₅-C₁₃ alkyl group; and R² represents a linear alkyl group with a carbon number corresponding to the carbon number of R¹ from which two carbon atoms are subtracted, and R³ represents a linear alkyl group with a carbon number corresponding to the carbon number of R¹ from which four carbon atoms are subtracted; and
preferably, m<n+p when R¹ represents a substituted and/or interrupted alkyl group, an optionally substituted and/or optionally interrupted alkenyl group or an optionally substituted and/or optionally interrupted alkynyl group, and R² and R³ represent an alkyl group.

4. Composition according to any one of the preceding claims, in which the PHA copolymer(s) a) contain four different repeating polymer units (A), (B), (C) and (D), and preferably consist of four different polymer units (A), (B), (C) and (D), below, and also the optical or geometrical isomers thereof, the organic or mineral acid or base salts thereof, and also the solvates thereof such as hydrates:
—[—O—CH(R¹)—CH₂—C(O)—]— unit (A)
—[—O—CH(R²)—CH₂—C(O)—]— unit (B)
—[—O—CH(R³)—CH₂—C(O)—]— unit (C)
—[—O—CH(R⁴)—CH₂—C(O)—]— unit (D)
in which polymer units (A), (B), (C) and (D):
R¹, R² and R³ are as defined in any one of claims 1 to 4;
R⁴ represents a cyclic or non-cyclic, linear or branched, saturated hydrocarbon-based group comprising from 3 to 30 carbon atoms optionally substituted with one or more atoms or groups a) to m) and/or optionally interrupted with one or more heteroatoms or groups a′) to c′) as defined for R¹ in any one of the preceding claims; it in particular represents a hydrocarbon-based group chosen from linear or branched (C₄-C₂₈)alkyl optionally substituted with one or more atoms or groups a) to m) and/or interrupted with one or more heteroatoms or groups a′) to c′) as defined for R¹ as defined previously; and
it being understood that:
(A) is different from (B), (C) and (D), (B) is different from (A), (C) and (D), (C) is different from (A), (B) and (D), and (D) is different from (A), (B) and (C);
preferably, when R¹, R², R³ and R⁴ represent an unsubstituted and uninterrupted alkyl group, the molar percentage of units (A) is greater than the molar percentage of units (B), greater than the molar percentage of units (C), and greater than the molar percentage of units (D)—more preferentially, when R¹, R², R³ and R⁴ are linear alkyl, then R¹ is a C₅-C₁₃ alkyl group; and R² represents a linear alkyl group with a carbon number corresponding to the carbon number of R¹ from which two carbon atoms are subtracted, R³ represents a linear alkyl group with a carbon number corresponding to the carbon number of R¹ from which four carbon atoms are subtracted, and R⁴ represents a linear alkyl group with a carbon number corresponding to the carbon number of R¹ from which six carbon atoms are subtracted; and
preferably, when R¹ represents a substituted and/or interrupted alkyl, optionally substituted and/or optionally interrupted alkenyl or optionally substituted and/or optionally interrupted alkynyl group, then the molar percentage of units (A) is less than the molar percentage of units (B) and less than the molar percentage of units (C), notably if R² represents an alkyl group and/or R³ represents an alkyl group; and
R⁴ represents a substituted and/or interrupted alkyl, optionally substituted and/or optionally interrupted alkenyl or optionally substituted and/or optionally interrupted alkynyl group;
more preferentially, the PHA copolymer(s) comprise the repeating unit of formula (III), and also the optical or geometrical isomers thereof, the organic or mineral acid or base salts thereof, and the solvates thereof such as hydrates: 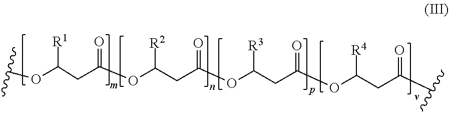
in which formula (III):
R¹, R², R³ and R⁴ are as defined previously;
m, n, p and v are integers greater than or equal to 1; preferably, the sum n+m+p+v is inclusively between 450 and 1400;
preferably, when R¹, R², R³ and R⁴ represent an unsubstituted and uninterrupted alkyl group, then m>n+p+q—more preferentially, when R¹, R², R³ and R⁴ are linear alkyl, then R¹ is a C₅-C₁₃ alkyl group; and R² represents a linear alkyl group with a carbon number corresponding to the carbon number of R¹ from which two carbon atoms are subtracted, R³ represents a linear alkyl group with a carbon number corresponding to the carbon number of R¹ from which four carbon atoms are subtracted, and R⁴ represents a linear alkyl group with a carbon number corresponding to the carbon number of R¹ from which six carbon atoms are subtracted; and
preferably, when R¹ represents a substituted and/or interrupted alkyl, optionally substituted and/or optionally interrupted alkenyl or optionally substituted and/or optionally interrupted alkynyl group, and R² and R³ represent an alkyl group, and R⁴ represents a substituted and/or interrupted alkyl, optionally substituted and/or optionally interrupted alkenyl or optionally substituted and/or optionally interrupted alkynyl group, then n>m+v; more preferentially n+p>m+v.

5. Composition according to any one of the preceding claims, in which the PHA copolymer(s) a) contain five different repeating polymer units (A), (B), (C), (D) and (E), and preferably consist of five different polymer units (A), (B), (C), (D) and (E), below, and also the optical or geometrical isomers thereof, the organic or mineral acid or base salts thereof, and also the solvates thereof such as hydrates:
—[—O—CH(R¹)—CH₂—C(O)—]— unit (A)
—[—O—CH(R²)—CH₂—C(O)—]— unit (B)
—[—O—CH(R³)—CH₂—C(O)—]— unit (C)
—[—O—CH(R⁴)—CH₂—C(O)—]— unit (D)
—[—O—CH(R⁵)—CH₂—C(O)—]— unit (E)
in which polymer units (A), (B), (C), (D) and (E):
R¹, R², R³ and R⁴ are as defined in any one of the preceding claims; and
R⁵ represents a cyclic or non-cyclic, linear or branched, saturated hydrocarbon-based group comprising from 3 to 30 carbon atoms optionally substituted with one or more atoms or groups a) to m) and/or optionally interrupted with one or more heteroatoms or groups a′) to c′) as defined for R¹; it in particular represents a hydrocarbon-based group chosen from linear or branched (C₄-C₂₅)alkyl substituted with one or more atoms or groups a) to l) and/or interrupted with one or more heteroatoms or groups a′) to c′) as defined for R¹; preferably, the hydrocarbon-based group has a carbon number corresponding to the number of carbon atoms of the radical R⁴ from which at least one carbon atom is subtracted, preferably corresponding to the number of carbon atoms of the radical R⁴ from which at least two carbon atoms are subtracted, preferably from which two carbon atoms are subtracted;
it being understood that:
(A) is different from (B), (C), (D) and (E); (B) is different from (A), (C), (D) and (E); (C) is different from (A), (B), (D) and (E); (D) is different from (A), (B), (C) and (E);
and (E) is different from (A), (B), (C) and (D);
preferably, when R¹, R², R³, R⁴ and R⁵ represent an unsubstituted and uninterrupted alkyl group, the molar percentage of units (A) is greater than the molar percentage of units (B), greater than the molar percentage of units (C), greater than the molar percentage of units (D) and greater than the molar percentage of units (E)—more preferentially, when R¹, R², R³, R⁴ and R⁵ are linear alkyl, then R¹ is a C₅-C₁₃ alkyl group; and R² represents a linear alkyl group with a carbon number corresponding to the carbon number of R¹ from which two carbon atoms are subtracted, R³ represents a linear alkyl group with a carbon number corresponding to the carbon number of R¹ from which four carbon atoms are subtracted, R⁴ represents a linear alkyl group with a carbon number corresponding to the carbon number of R¹ from which six carbon atoms are subtracted, and R⁵ represents a linear alkyl group with a carbon number corresponding to the carbon number of R¹ from which eight carbon atoms are subtracted, and
preferably, when R¹ represents a substituted and/or interrupted alkyl, optionally substituted and/or optionally interrupted alkenyl or optionally substituted and/or optionally interrupted alkynyl group, then the molar percentage of units (A) is less than the molar percentage of units (B) and less than the molar percentage of units (C), notably if R² represents an alkyl group and/or R³ represents an alkyl group; and R⁴ and R⁵ represent a substituted and/or interrupted alkyl, optionally substituted and/or optionally interrupted alkenyl or optionally substituted and/or optionally interrupted alkynyl group;
more preferentially, the PHA copolymer(s) comprise the repeating unit of formula (IV), and also the optical or geometrical isomers thereof, the organic or mineral acid or base salts thereof, and the solvates thereof such as hydrates: 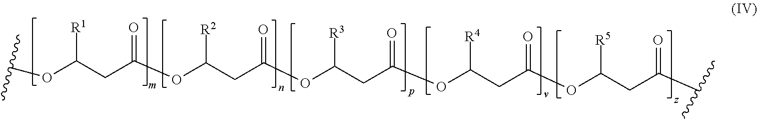
in which formula (IV):
R¹, R², R³, R⁴ and R⁵ are as defined previously;
m, n, p, v and z are integers greater than or equal to 1; preferably, the sum n+m+p+v+z is inclusively between 450 and 1400;
preferably, when R¹, R², R³, R⁴ and R⁵ represent an unsubstituted and uninterrupted alkyl group, then m>n+p+v+z;
preferably, when R¹ represents a substituted and/or interrupted alkyl; optionally substituted and/or optionally interrupted alkenyl; or optionally substituted and/or optionally interrupted alkynyl group, R² and R³ represent an alkyl group, and the groups R⁴ and R⁵ represent a substituted and/or interrupted alkyl; optionally substituted and/or optionally interrupted alkenyl; or optionally substituted and/or optionally interrupted alkynyl group, then n>m+v+z; more preferentially n+p>m+v+z.

6. Composition according to any one of the preceding claims, in which R¹ represents a linear or branched, preferably linear, (C₅-C₂₈)alkyl hydrocarbon-based chain; more particularly, R¹ is an alkyl group substituted with one or more atoms or groups a) to k), said alkyl group comprising from 5 to 12, preferably between 6 and 10 carbon atoms, more preferentially between 7 and 9 carbon atoms such as n-octyl; preferably, R¹ represents a hydrocarbon-based chain, substituted with one or more (preferably one) groups chosen from b) hydroxyl, c) thiol, d) (di)(C₁-C₄)(alkyl)amino, preferably amino, e) carboxyl, i) (hetero)cycloalkyl such as anhydride, or epoxide, j) a cosmetic active agent chosen from coloured or uncoloured, fluorescent or non-fluorescent chromophores such as optical brighteners, UV-screening agents, h) (hetero)aryl such as phenyl or furyl, k) R—X with R representing a group chosen from α) cycloalkyl such as cyclohexyl, β) heterocycloalkyl such as a sugar, preferably a monosaccharide such as glucose, γ) (hetero)aryl such as phenyl, δ) a cosmetic active agent as defined previously and X representing a′) O, S, N(Rₐ), b′) carbonyl, c′) or combinations thereof of a′) with b′) such as ester, amide or urea; Rₐ represents a hydrogen atom or a (C₁-C₄)alkyl or aryl(C₁-C₄)alkyl group such as benzyl, preferably Rₐ represents a hydrogen atom; even more preferentially, the PHA copolymer(s) are such that R¹ represents a hydrocarbon-based chain, notably an alkyl group as defined previously, which is substituted with one or more (preferably one) groups chosen from a) halogen such as chlorine or bromine, b) hydroxyl, d) (di)(C₁-C₄)(alkyl)amino, preferably amino, e) carboxyl, i) (hetero)cycloalkyl such as epoxide, h) (hetero)aryl such as phenyl or furyl, k) R—X with R representing a group chosen from α) cycloalkyl such as cyclohexyl, β) heterocycloalkyl such as a sugar, preferably a monosaccharide such as glucose, γ) (hetero)aryl such as phenyl, and X representing a′) O, S or N(Rₐ), preferably S; Rₐ representing a hydrogen atom or a (C₁-C₄)alkyl group, preferably Rₐ represents a hydrogen atom;
better still, said hydrocarbon-based chain R¹ is substituted at the end of the chain on the opposite side from the carbon atom which bears said radical R¹.

7. Composition according to any one of the preceding claims, in which R¹ has the following formula —(CH₂)ᵣ—X-(ALK)ᵤ-G with X being as defined in any one of the preceding claims, in particular representing O, S or N(Rₐ), preferably S, ALK represents a linear or branched, preferably linear, (C₁-C₁₀)alkylene and more particularly (C₁-C₈)alkylene chain, r represents an integer inclusively between 6 and 11, preferably between 7 and 10 such as 8; u is equal to 0 or 1; and G represents a hydrogen atom or a group chosen from hydroxyl, carboxyl, (di)(C₁-C₄)(alkyl)amino, (hetero)aryl in particular aryl such as phenyl, cycloalkyl such as cyclohexyl, or a sugar, in particular a monosaccharide optionally protected with one or more groups such as acyl, preferably Sug represents 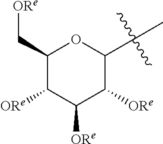
with R^{e} representing a group R^{f}—C(O)—, with R^{f} representing a (C₁-C₄)alkyl group such as methyl; preferably, when u is equal to 0, G represents a cycloalkyl group such as cyclohexyl, or a sugar as defined previously; according to another advantageous variant, when u is equal to 1, G represents a hydrogen atom or a group chosen from hydroxyl, carboxyl, (di)(C₁-C₄)(alkyl)amino or (hetero)aryl, in particular aryl such as phenyl.

8. Composition according to any one of the preceding claims, in which the PHA copolymer(s) a) include a radical R² chosen from linear or branched (C₃-C₂₀)alkyl, and linear or branched (C₃-C₂₀)alkenyl, in particular a linear hydrocarbon-based group; preferably, the hydrocarbon-based group has a carbon number corresponding to the number of carbon atoms of the radical R¹ from which at least one carbon atom is subtracted, preferably corresponding to the number of carbon atoms of the radical R¹ from which at least two carbon atoms are subtracted.

9. Composition according to any one of the preceding claims, in which the PHA copolymer(s) a) include a radical R² which is a linear or branched, preferably linear, (C₄-C₆)alkyl, preferably a branched (C₄-C₅)alkyl such as isobutyl.

10. Composition according to any one of claims 3 to 9, in which the PHA copolymer(s) are such that, when R¹ represents a (C₅-C₂₈)alkyl group, the unit (A) is present in a molar percentage ranging from 0.5% to 99%, more preferentially from 50% to 99%, more particularly from 60% to 99%, even more preferentially from 70% to 99%; the unit (B) is present in a molar percentage ranging from 0.5% to 40% by weight, preferably ranging from 2% to 40%, the unit (C) is present in a molar percentage ranging from 0.5% to 20% relative to all the units (A), (B) and (C).

11. Composition according to any one of claims 3 to 9, in which the PHA copolymer(s) a) are such that when R¹ represents a hydrocarbon-based chain chosen from i) linear or branched (C₅-C₂₈)alkyl, ii) linear or branched (C₅-C₂₈)alkenyl, iii) linear or branched (C₅-C₂₈)alkynyl, the hydrocarbon-based group is preferably linear, said hydrocarbon-based chain being substituted with one or more atoms or groups a) to m) and/or interrupted with one or more heteroatoms or groups a′) to c′) as defined for R¹;
the unit (A) is present in a molar percentage ranging from 0.1% to 99%, preferentially a molar percentage ranging from 0.5% to 50%, more preferentially a molar percentage ranging from 1% to 40%, even more preferentially a molar percentage ranging from 2% to 30%, better still a molar percentage ranging from 5% to 30%, better still a molar percentage ranging from 10 mol % to 30 mol % of units (A); and
the unit (B) is present in a molar percentage ranging from 1% to 99.5%, preferably from 1% to 90%, more preferentially a molar percentage from 2% to 70%, and
the unit (C) is present in a molar percentage ranging from 0.5% to 20%, relative to all the units (A), (B) and (C); advantageously, the PHA copolymer(s) of the invention comprise from 2 mol % to 70 mol % of units (B);
and from 0.5 mol % to 7 mol % of units (C); more advantageously, the copolymer comprises from 5 mol % to 35 mol % of units (B); and from 0.5 mol % to 7 mol % of units (C).

12. Composition according to any one of the preceding claims, in which the unit(s) (A) of the PHA copolymer(s) are chosen from the following repeating units (A), and also the optical or geometrical isomers thereof, the organic or mineral acid or base salts thereof, and the solvates thereof such as hydrates:

| (A) | R¹ |
|---|---|
| A1 | —ALK₁—S—ALK₂—COOH |
| A2 | —ALK₁—S—ALK₂—H |
| A3 | —ALK₁—S—ALK₂—OH |
| A4 | —ALK₁—S—ALK₂—NH₂ |
| A5 | —ALK₁—S—Cycl′ |
| A6 | —ALK₁—S—CH₂—Fur |
| A7 | —ALK₁—S—Sug |
| A8 | —ALK₁—S—ALK₂—Ar |
| A9 | —ALK₁—Hal |
| A10 | —ALK₁—CN |
| A11 | —ALK₁—CH═CRᵣR_{w} |
| A12 | —ALK₂—H |

in which repeating units A1 to A12:
ALK₁ represents a divalent linear or branched C₁-C₂₀, preferably linear or branched, more preferentially linear, C₁-C₁₀, hydrocarbon-based radical;
ALK₂ represents a divalent linear or branched C₁-C₂₀, preferably linear or branched C₁-C₁₂, hydrocarbon-based radical;
Rr and Rw independently denote a hydrogen atom or a C₁-C₄ alkyl radical such as methyl; preferably, Rr and Rw are identical;
Hal represents a halogen atom such as bromine;
Ar: represents a (hetero)aryl group such as phenyl;
Cycl′: represents a cycloalkyl group such as cyclohexyl or heterocycloalkyl such as dithiolane, or epoxide, preferably epoxide;
Fur: represents a furyl group, preferably 2-furyl;
Sug: represents a sugar group, in particular a monosaccharide optionally protected with one or more groups such as acyl, in particular acetyl;
in particular, the stereochemistry of the carbon atoms bearing the radicals R¹ is of (R) configuration.

13. Composition according to any one of the preceding claims, in which the PHA copolymer(s) a) comprise the following repeating units, and also the optical or geometrical isomers thereof, the organic or mineral acid or base salts thereof, and the solvates thereof such as hydrates:

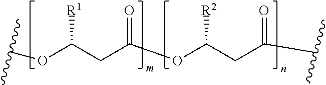

| Compounds | R¹ | R² |
|---|---|---|
| (1) | —(CH₂)₈—S—CH(CH₃)—C(O)—OH | —(CH₂)₄—CH₃ |
| (2) | —(CH₂)₈—S—(CH₂)₇—CH₃ | —(CH₂)₄—CH₃ |
| (3) | —(CH₂)₈—S—(CH₂)₈—OH | —(CH₂)₄—CH₃ |
| (4) | —(CH₂)₈—S—(CH₂)₂—NH₂ | —(CH₂)₄—CH₃ |
| (5) | —(CH₂)₈—S-Cycl | —(CH₂)₄—CH₃ |
| (6) | —(CH₂)₈—S—CH₂-Fur | —(CH₂)₄—CH₃ |
| (7) | —(CH₂)₈—S-Sug | —(CH₂)₄—CH₃ |
| (8) | —(CH₂)₈—S—(CH₂)₂—Ar | —(CH₂)₄—CH₃ |
| (9) | —(CH₂)₈—S—CH₂—Ar′ | —(CH₂)₄—CH₃ |
| (10) | —(CH₂)₈—S—CH(CH₃)—C(O)—OH | —(CH₂)₅—CH₃ |
| (11) | —(CH₂)₅-Hal | —(CH₂)₅—CH₃ |
| (12) | —(CH₂)₃—CN | —(CH₂)₅—CH₃ |
| (13) | 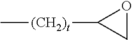 | —(CH₂)₅—CH₃ |
| (14) | —(CH₂)₂—Ar | —(CH₂)₅—CH₃ |
| (15) | —(CH₂)₄—CH₃ | —(CH₂)₂—CH₃ |
| (16) | —(CH₂)₅—CH₃ | —(CH₂)₃—CH₃ |
| (17) | —(CH₂)₆—CH₃ | —(CH₂)₄—CH₃ |
| (18) | —(CH₂)₈—CH₃ | —(CH₂)₆—CH₃ |
| (19) | —(CH₂)₃—CH(CH₃)CH₃ | —CH₂—CH(CH₃)CH₃ |
| (20) | —(CH₂)₆—CH═CH₂ | —(CH₂)₅—CH₃ |
| (21) | —(CH₂)₂—CH═C(CH₃)CH₃ | —CH₂—CH(CH₃)CH₃ |
| (2a) | —(CH₂)₈—S—(CH₂)₇—CH₃ | (CH₂)₅—(CH₃) |

m and n are as defined in claim 2, Hal represents a halogen atom such as bromine and t represents an integer between 1 and 10, preferably between 3 and 8 such as 6.
Ar: represents a (hetero)aryl group such as phenyl;
Ar′: represents a (C₁-C₄)alkyl(hetero)aryl group such as t-butylphenyl, preferably 4-t-butylphenyl;
Cycl: represents a cyclohexyl group;
Fur: represents a furyl group, preferably 2-furyl;
Sug: represents a sugar group, in particular a monosaccharide optionally protected with one or more groups such as acyl; preferably, Sug represents: 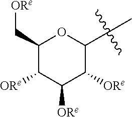
with R^{e} representing a group R^{f}—C(O)—, with R^{f} representing a (C₁-C₄)alkyl group such as methyl,
more preferentially, the PHA copolymer(s) have the following formula, and also the optical isomers thereof, the organic or mineral acid or base salts thereof, and the solvates thereof such as hydrates:

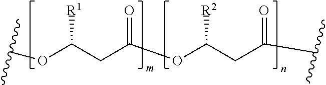

| Compounds | R¹ | R² |
|---|---|---|
| (1′) | —(CH₂)₈—S—CH(CH₃)—C(O)—OH | —(CH₂)₄—CH₃ |
| (2′) | —(CH₂)₈—S—(CH₂)₇—CH₃ | —(CH₂)₄—CH₃ |
| (3′) | —(CH₂)₈—S—(CH₂)₈—OH | —(CH₂)₄—CH₃ |
| (4′) | —(CH₂)₈—S—(CH₂)₂—NH₂ | —(CH₂)₄—CH₃ |
| (5′) | —(CH₂)₈—S-Cycl | —(CH₂)₄—CH₃ |
| (6′) | —(CH₂)₈—S—CH₂-Fur | —(CH₂)₄—CH₃ |
| (7′) | —(CH₂)₈—S-Sug | —(CH₂)₄—CH₃ |
| (8′) | —(CH₂)₈—S—(CH₂)₂—Ar | —(CH₂)₄—CH₃ |
| (9′) | —(CH₂)₈—S—CH₂—Ar′ | —(CH₂)₄—CH₃ |
| (10′) | —(CH₂)₈—S—CH(CH₃)—C(O)—OH | —(CH₂)₅—CH₃ |
| (11′) | —(CH₂)₅-Hal | —(CH₂)₅—CH₃ |
| (12′) | —(CH₂)₃—CN | —(CH₂)₅—CH₃ |
| (13′) | 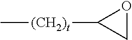 | —(CH₂)₅—CH₃ |
| (14′) | —(CH₂)₂—Ar | —(CH₂)₅—CH₃ |
| (15′) | —(CH₂)₄—CH₃ | —(CH₂)₂—CH₃ |
| (16′) | —(CH₂)₅—CH₃ | —(CH₂)₃—CH₃ |
| (17′) | —(CH₂)₆—CH₃ | —(CH₂)₄—CH₃ |
| (18′) | —(CH₂)₈—CH₃ | —(CH₂)₆—CH₃ |
| (19′) | —(CH₂)₃—CH(CH₃)CH₃ | —CH₂—CH(CH₃)CH₃ |
| (20′) | —(CH₂)₆—CH═CH₂ | —(CH₂)₅—CH₃ |
| (21′) | —(CH₂)₂—CH═C(CH₃)CH₃ | —CH₂—CH(CH₃)CH₃ |
| (22′) | —(CH₂)₄—CH₃ | —(CH₂)₂—CH₃ |
| (23′) | —(CH₂)₅—CH₃ | —(CH₂)₃—CH₃ |
| (24′) | —(CH₂)₆—CH₃ | —(CH₂)₄—CH₃ |
| (2a′) | —(CH₂)₈—S—(CH₂)₇—CH₃ | (CH₂)₅—(CH₃) |

n, Hal, t, Ar, Ar′, Cycl, Fur and Sug are as defined previously for compounds (1) to (14).

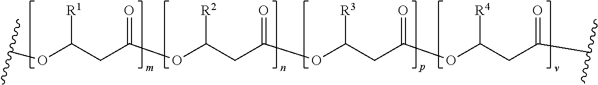

| Compounds | R¹ | R² | R³ | R⁴ |
|---|---|---|---|---|
| (25) | —(CH₂)₈—CH₃ | —(CH₂)₆—CH₃ | —(CH₂)₄—CH₃ | —(CH₂)₂—CH₃ |
| (26) | —(CH₂)₆—CH₃ | —(CH₂)₄—CH₃ | —(CH₂)₂—CH₃ | —CH₃ |
| (27) | —(CH₂)₂—CN | —(CH₂)₅—CH₃ | —(CH₂)₃—CH₃ | —CN |
| (28) | —(CH₂)₂—Ar | —(CH₂)₅—CH₃ | —(CH₂)₃—CH₃ | —Ar |

with m, n, p and v as being as defined in claim 4, and Ar as being defined previously,

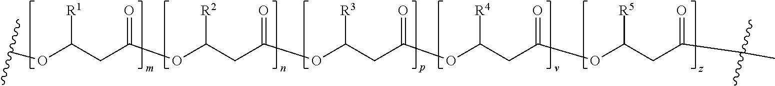

| Com- | | | | | |
|---|---|---|---|---|---|
| pounds | R¹ | R² | R³ | R⁴ | R⁵ |
| (29) | —(CH₂)₉—CH═CH₂ | —(CH₂)₅—CH₃ | —(CH₂)₃—CH₃ | —(CH₂)₇—CH═CH₂ | —(CH₂)₅—CH═CH₂ |
| (30) | —(CH₂)₉—CH═CH₂ | —(CH₂)₄—CH₃ | —(CH₂)₂—CH₃ | —(CH₂)₇—CH═CH₂ | —(CH₂)₅—CH═CH₂ |
| (31) | —(CH₂)₈—CH₃ | —(CH₂)₆—CH₃ | —(CH₂)₄—CH₃ | —(CH₂)₂—CH₃ | —CH₃ |
| (32) | —(CH₂)₁₀—CH₃ | —(CH₂)₆—CH₃ | —(CH₂)₆—CH₃ | —(CH₂)₄—CH₃ | —(CH₂)₂—CH₃ |
| (33) | —(CH₂)₈—S—CH(CH₃)— | —(CH₂)₄—CH₃ | —(CH₂)₂—CH₃ | —(CH₂)₆—S—CH(CH₃)— | —(CH₂)₄—S—CH(CH₃)— |
| | C(O)—OH | | | C(O)—OH | C(O)—OH |
| (34) | —(CH₂)₈—S—(CH₂)₇—CH₃ | —(CH₂)₄—CH₃ | —(CH₂)₂—CH₃ | —(CH₂)₆—S—(CH₂)₇—CH₃ | —(CH₂)₄—S—(CH₂)₇—CH₃ |
| (35) | —(CH₂)₈—S—(CH₂)₈—OH | —(CH₂)₄—CH₃ | —(CH₂)₂—CH₃ | —(CH₂)₆—S—(CH₂)₈—OH | —(CH₂)₄—S—(CH₂)₈—OH |
| (36) | —(CH₂)₈—S—(CH₂)₂—NH₂ | —(CH₂)₄—CH₃ | —(CH₂)₂—CH₃ | —(CH₂)₆—S—(CH₂)₂—NH₂ | —(CH₂)₄—S—(CH₂)₂—NH₂ |
| (37) | —(CH₂)₈—S-Cycl | —(CH₂)₄—CH₃ | —(CH₂)₂—CH₃ | —(CH₂)₆—S-Cycl | —(CH₂)₄—S-Cycl |
| (38) | —(CH₂)₈—S—CH₂-Fur | —(CH₂)₄—CH₃ | —(CH₂)₂—CH₃ | —(CH₂)₆—S—CH₂-Fur | —(CH₂)₄—S—CH₂-Fur |
| (39) | —(CH₂)₈ [symbol] —S-Sug | —(CH₂)₄—CH₃ | —(CH₂)₂—CH₃ | —(CH₂)₆ —S-Sug | —(CH₂)₄—S-Sug |
| (40) | —(CH₂)₈—S—(CH₂)₂—Ar | —(CH₂)₄—CH₃ | —(CH₂)₂—CH₃ | —(CH₂)₆—S—(CH₂)₂—Ar | —(CH₂)₄—S—(CH₂)₂—Ar |
| (41) | —(CH₂)₈—S—CH₂—Ar′ | —(CH₂)₄—CH₃ | —(CH₂)₂—CH₃ | —(CH₂)₆—S—CH₂—Ar′ | —(CH₂)₄—S—CH₂—Ar′ |
| (42) | —(CH₂)₈—S—CH(CH₃)— | —(CH₂)₅—CH₃ | —(CH₂)₃—CH₃ | —(CH₂)₆—S—CH(CH₃)— | —(CH₂)₄—S—CH(CH₃)— |
| | C(O)—OH | | | C(O)—OH\| | C(O)—OH |
| (43) | —(CH₂)₅-Hal | —(CH₂)₅—CH₃ | —(CH₂)₃—CH₃ | —(CH₂)₃-Hal | —(CH₂)-Hal |
| (44) | 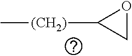 | —(CH₂)₅—CH₃ | —(CH₂)₃—CH₃ | 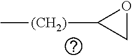 | 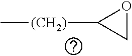 |
| (34a) | —(CH₂)₈—S—(CH₂)₇—CH₃ | —(CH₂)₅—(CH₃) | —(CH₂)₃—(CH₃) | —(CH₂)₆—S—(CH₂)₇—CH₃ | —(CH₂)₄—S—(CH₂)₇—CH₃ |

[symbol]

indicates data missing or illegible when filed
with m, n, p, v and z as being defined in claim 5, and Hal, t, Ar, Ar′, Cycl, Fur and Sug being as defined previously for compounds (1) to (14),
preferably, the PHA(s) of the invention are chosen from compounds (15), (16) and (17), notably (16); more particularly, the PHA(s) of the invention are chosen from compounds (15′), (16′) and (17′), notably (16′); more preferentially, the PHA(s) a) of the invention are chosen from compounds (25), (26), (31) and (32), notably (26).

14. Composition according to any one of the preceding claims, in which the PHA copolymer(s) a) are in a content ranging from 0.1% to 65% by weight, preferably from 0.1% to 60%, particularly from 1% to 50% by weight, more particularly from 3% to 40% by weight, more preferably from 5% to 35% by weight, even more preferably from 5% to 30%, better ranging from 5% to 20% by weight relative to the total weight of the composition.

15. Composition according to any one of the preceding claims, in which b) the resin(s) are chosen from: a) acaroid resins, b) ambers, c) asphaltite and gilsonite,
d) Peruvian balsam, e) Tolu balsam, f) benzoin resins, g) Canada balsam, h) copal resins (notably Kauri copal resins, Manila, West African copals such as Congo, Angola or Cameroon copals, East African copals such as Zanzibar or Madagascar copals, South American copals such as Brazilian or Colombian copals), i) dammar resins, j) elemi resins, k) frankincense, l) galbanum, m) labdanum, n) mastic, o) myrrh, p) sandarac, q) shellac, r) Styrax (storax), s) Venice turpentine (larch, turpentine oil), t) rosin, notably rosin and rosinate, and tall oils; v) resins extracted from plant waxes such as candelilla resin, preferably the natural resin(s) are chosen from j), k), t) and v); it is understood that the resin(s) b) may be esterified, salified, adducted, modified with phenols, and/or dimerized and also hydrogenated.

16. Composition according to any one of the preceding claims, in which b) the b) the resin(s) are chosen from:
j) elemi resins, notably elemi from the Canarium luzonicum family in its pure form or mixed with a latex for example, notably Canarium luzonicum,
k) frankincense resins, notably Amazonian frankincense resins or Protium heptaphyllum resin, and frankincense resins from the sal tree, Shorea robusta; the resin(s) may be in a mixture with one or more fatty substances c), preferably chosen from volatile or nonvolatile oils such as Shorea robusta resin and sunflower oil, and tocopherol; and Shorea robusta resin with octyldodecanol;
(t) colophony resins notably:
rosin acid esters (rosin) in particular esters of rosin acid and of (C₁-C₆) alkanol, polyhydroxy(C₁-C₆)alkane polyols such as glycerol, or pentaerythritol, more preferentially chosen from glyceryl rosinate, pentaerythrityl rosinate and glyceryl rosinate as a mixture with a hydrogenated plant oil and/or castor seed oil;
hydrogenated rosins; particularly chosen from pentaerythrityl hydrogenated rosinate, methyl hydrogenated rosinate; or mixtures thereof with one or more fatty substances c) notably chosen from waxes or butters such as the mixture with shea butter or olive oil; and
v) resins extracted from plant waxes such as resin extracted from cadelilla wax.

17. Composition according to any one of the preceding claims, in which b):
the resin(s) are present in the composition in an amount of between 0.01% and 30% by weight relative to the total weight of the composition, more particularly between 0.1% and 20% by weight relative to the total weight of the composition.

18. Composition according to any one of the preceding claims, which comprises
c) one or more fatty substances, which are preferably liquid at 25° C. and at atmospheric pressure, particularly chosen from:
hydrocarbons, in particular alkanes, oils of animal origin,
oils of plant origin,
glycerides or fluoro oils of synthetic origin,
fatty alcohols,
esters of a fatty acid and/or of a fatty alcohol,
non-silicone waxes, silicones; and
mixtures thereof;
in particular, the liquid hydrocarbon-based fatty substance(s) are hydrocarbon-based oils, which are preferably volatile, or are a mixture of different volatile oils;
the volatile oil(s) are preferably chosen from linear or branched C₈-C₁₄, more preferentially C₉-C₁₃ and even more preferentially C₉-C₁₂ alkanes, more particularly chosen from undecane, dodecane, tridecane and isododecane; or a mixture of different volatile oils preferably comprising isododecane in the mixture, or a mixture of undecane and tridecane.

19. Composition according to any one of the preceding claims, which comprises
c) one or more fatty substances, which are preferably liquid at 25° C. and at atmospheric pressure, particularly chosen from:
plant oils formed by fatty acid esters of polyols, in particular triglycerides, such as sunflower oil, sesame oil, rapeseed oil, macadamia oil, soybean oil, sweet almond oil, beauty-leaf oil, palm oil, grapeseed oil, corn oil, arara oil, cottonseed oil, apricot oil, avocado oil, jojoba oil, olive oil or cereal germ oil;
linear, branched or cyclic esters containing more than 6 carbon atoms, notably 6 to 30 carbon atoms; and notably isononyl isononanoate;
and more particularly esters of formula R^{d}—C(O)—O—R^{e} in which R^{d} represents a higher fatty acid residue including from 7 to 19 carbon atoms and R^{e} represents a hydrocarbon-based chain including from 3 to 20 carbon atoms, such as palmitates, adipates, myristates and benzoates, notably diisopropyl adipate and isopropyl myristate; more preferentially esters of formula R^{d}—C(O)—O—R^{e} in which R^{d} represents a higher fatty acid residue including from 8 to 10 carbon atoms and R^{e} represents a hydrocarbon-based chain including from 12 to 18 carbon atoms;
hydrocarbons and notably volatile or non-volatile linear, branched and/or cyclic alkanes, such as optionally volatile C₅-C₆₀ isoparaffins, such as isododecane, Parleam (hydrogenated polyisobutene), isohexadecane, cyclohexane, or Isopars, and mixtures thereof; or alkanes resulting from the complete hydrogenation/reduction of mixtures of fatty acids derived from Cocos nucifera (coconut) oil, such as dodecane; or the mixture of C₉-C₁₂ alkanes, the chains of which comprise from 9 to 12 carbon atoms, preferably linear or branched C₉-C₁₂ alkanes, in particular comprising dodecane, or else liquid paraffin, liquid petroleum jelly, or hydrogenated polyisobutylene;
ethers containing 6 to 30 carbon atoms;
ketones containing 6 to 30 carbon atoms;
aliphatic fatty monoalcohols containing 6 to 30 carbon atoms, the hydrocarbon-based chain not including any substitution groups, such as oleyl alcohol, decanol, dodecanol, octadecanol, octyldodecanol and linoleyl alcohol;
polyols containing 6 to 30 carbon atoms, such as hexylene glycol; and
mixtures thereof such as mixtures of esters of linear or branched C₈-C₁₀ fatty acid and C₁₂-C₁₈ fatty alcohol and alkanes resulting from the complete hydrogenation/reduction of fatty acid mixtures obtained from Cocos nucifera (coconut) oil, in particular dodecane, such as mixtures of cocoyl caprylate/caprate and dodecane; or mixtures of C₉-C₁₂ alkanes, the chains of which comprise from 9 to 12 carbon atoms, preferably linear or branched C₉-C₁₂ alkanes, in particular comprising dodecane; preferably the hydrocarbon-based liquid fatty substance(s) iii) are chosen from linear or branched C₆-C₁₆, preferably C₈-C₄, more preferentially C₉-C₁₃ and even more preferentially C₉-C₁₂ alkanes, and even more preferentially the alkanes are volatile and are chosen from undecane, dodecane, isododecane, tridecane, and a mixture thereof notably comprising dodecane, isododecane or a mixture of undecane and tridecane;
or else the liquid fatty substance(s) iii) are a mixture of non-volatile oil(s) and volatile oil(s), notably comprising, as volatile oil, isododecane, undecane, dodecane, isododecane and/or tridecane, more preferentially isododecane; as a mixture of volatile and non-volatile oil, mention may be made of the mixture of isododecane and isononyl isononanoate; and more preferentially, when the fatty substance(s) are a mixture of volatile and non-volatile oil, the amount of volatile oil is greater than the amount of non-volatile oil;
more preferentially fatty substance(s) c) are chosen from alkanes, such as undecane, tridecane, dodecane, decane, isododecane, hydrogenated polyisobutene, fatty alcohols such as octyldodecanol, esters such as isononyl isononanoate, cocoyl caprylate/caprate and mixtures thereof, even more preferentially from alkanes.

20. Composition according to any one of the preceding claims, comprising d) one or more organic solvents other than c) polar protic or polar aprotic organic solvents, preferably polar protic, particularly lower monoalcohols containing from 2 to 10 carbon atoms such as ethanol and isopropanol, preferably ethanol; preferably, the amount of organic solvent(s) is less than 70% by weight, more preferentially less than 50% by weight, relative to the total weight of the composition, preferably greater than 0.5%, more particularly greater than or equal to 1% by weight relative to the total weight of the composition; in particular, the composition comprises between 2% and 50% by weight of organic solvent(s).

21. Composition according to any one of the preceding claims, which also comprises one or more colouring agents chosen from pigments, direct dyes and mixtures thereof, preferably pigments; more preferentially, the pigment(s) of the invention are chosen from carbon black, iron oxides, notably black iron oxides, and micas coated with iron oxide, triarylmethane pigments, notably blue and violet triarylmethane pigments, such as Blue 1 Lake, azo pigments, notably red azo pigments, such as D&C Red 7, an alkali metal salt of lithol red, such as the calcium salt of lithol red B, even more preferentially red iron oxides.

22. Process for treating keratin materials, preferably α) keratin fibres, notably human keratin fibres such as the hair, or β) human skin, in particular the lips, by applying the composition as defined in any one of the preceding claims.

23. Cosmetic use of the composition according to any one of claims 1 to 21.
